SEQUENCE LISTING (115 sequences)

SEQ ID NO: 1 - DNA, 20 bp, Artificial Sequence
  Synthetic gRNA target sequence for dummysg1
atcgtttccgcttaacggcg

SEQ ID NO: 2 - DNA, 20 bp, Artificial Sequence
  Synthetic gRNA target sequence for dummysg2
aaacggtacgacagcgtgtg

SEQ ID NO: 3 - DNA, 20 bp, Artificial Sequence
  Synthetic gRNA target sequence for dummysg3
ccatcaccgatcgtgagcct

SEQ ID NO: 4 - DNA, 20 bp, Artificial Sequence
  Synthetic gRNA target sequence for ADORA2Asg1
ctatttgcggatcttcctgg

SEQ ID NO: 5 - DNA, 20 bp, Artificial Sequence
  Synthetic gRNA target sequence for ADORA2Asg2
aagcagttgatgatgtgtag

SEQ ID NO: 6 - DNA, 20 bp, Artificial Sequence
  Synthetic gRNA target sequence for ADORA2Asg3
tggcttggtgaccggcacga

SEQ ID NO: 7 - DNA, 20 bp, Artificial Sequence
  Synthetic gRNA target sequence for ANXA3sg1
agaaatcagagacattaaca

SEQ ID NO: 8 - DNA, 20 bp, Artificial Sequence
  Synthetic gRNA target sequence for ANXA3sg2
aaaatctccagatgtgtctg

SEQ ID NO: 9 - DNA, 20 bp, Artificial Sequence
  Synthetic gRNA target sequence for ANXA3sg3
tcacaccaaagtcctcagat

SEQ ID NO: 10 - DNA, 20 bp, Artificial Sequence
  Synthetic gRNA target sequence for ANXA3sg1
ctgagaggtcaaatgcacag

SEQ ID NO: 11 - DNA, 20 bp, Artificial Sequence
  Synthetic gRNA target sequence for ANXA3sg2
tttgcatcaaagactgctgg

SEQ ID NO: 12 - DNA, 20 bp, Artificial Sequence
  Synthetic gRNA target sequence for ANXA3sg3
gctattcagaaagcaatcag

SEQ ID NO: 13 - DNA, 20 bp, Artificial Sequence
  Synthetic gRNA target sequence for CFTRsg1
aacgtgttgagggttgacat

SEQ ID NO: 14 - DNA, 20 bp, Artificial Sequence
  Synthetic gRNA target sequence for CFTRsg2
tgtggacagtaatatatcga

SEQ ID NO: 15 - DNA, 20 bp, Artificial Sequence
  Synthetic gRNA target sequence for CFTRsg3
ccacgcttcaggcacgaagg

SEQ ID NO: 16 - DNA, 20 bp, Artificial Sequence
  Synthetic gRNA target sequence for DPP4sg1
ggattccaaacaacacacag

SEQ ID NO: 17 - DNA, 20 bp, Artificial Sequence
  Synthetic gRNA target sequence for DPP4sg2
ctacttgtgtgatgtgacat

SEQ ID NO: 18 - DNA, 20 bp, Artificial Sequence
  Synthetic gRNA target sequence for DPP4sg3
atttattacaaaaggcacct

SEQ ID NO: 19 - DNA, 20 bp, Artificial Sequence
  Synthetic gRNA target sequence for FGAsg1
ggttgatatgaaacgactgg

SEQ ID NO: 20 - DNA, 20 bp, Artificial Sequence
  Synthetic gRNA target sequence for FGAsg2
acagtcagaaccatcttcgg

SEQ ID NO: 21 - DNA, 20 bp, Artificial Sequence
  Synthetic gRNA target sequence for FGAsg3
tgatccggttccataagagg

SEQ ID NO: 22 - DNA, 20 bp, Artificial Sequence
  Synthetic gRNA target sequence for FGFR2sg1
cttagtccaactgatcacgg

SEQ ID NO: 23 - DNA, 20 bp, Artificial Sequence
  Synthetic gRNA target sequence for FGFR2sg2
tgaccaaacgtatccccctg

SEQ ID NO: 24 - DNA, 20 bp, Artificial Sequence
  Synthetic gRNA target sequence for FGFR2sg3
gccggcaaatgcctccacag

SEQ ID NO: 25 - DNA, 20 bp, Artificial Sequence
  Synthetic gRNA target sequence for FGFR3sg1
aagaacggcagggagttccg

SEQ ID NO: 26 - DNA, 20 bp, Artificial Sequence
  Synthetic gRNA target sequence for FGFR3sg2
ggtgctgaatgcctcccacg

SEQ ID NO: 27 - DNA, 20 bp, Artificial Sequence
  Synthetic gRNA target sequence for FGFR3sg3
catccggcagacgtacacgc

SEQ ID NO: 28 - DNA, 20 bp, Artificial Sequence
  Synthetic gRNA target sequence for FLT4sg1
ctcacctctcacgaacacgt

SEQ ID NO: 29 - DNA, 20 bp, Artificial Sequence
  Synthetic gRNA target sequence for FLT4sg2
gccctccagtcacggcactg

SEQ ID NO: 30 - DNA, 20 bp, Artificial Sequence
  Synthetic gRNA target sequence for FLT4sg3
cataccatgcacaatgacct

SEQ ID NO: 31 - DNA, 20 bp, Artificial Sequence
  Synthetic gRNA target sequence for NMDAR1sg1
agccgttccagagcacactg

SEQ ID NO: 32 - DNA, 20 bp, Artificial Sequence
  Synthetic gRNA target sequence for NMDAR1sg2
caaaagccgtagcaacactg

SEQ ID NO: 33 - DNA, 20 bp, Artificial Sequence
  Synthetic gRNA target sequence for NMDAR1sg3
cgggcaggcagacatgatcg

SEQ ID NO: 34 - DNA, 20 bp, Artificial Sequence
  Synthetic gRNA target sequence for NMDAR3Bsg1
ccatgacattgtgcaactgg

SEQ ID NO: 35 - DNA, 20 bp, Artificial Sequence
  Synthetic gRNA target sequence for NMDAR3Bsg2
ggggctacgccactcgtaca

SEQ ID NO: 36 - DNA, 20 bp, Artificial Sequence
  Synthetic gRNA target sequence for NMDAR3Bsg3
aggtacagctcgaagtcgaa

SEQ ID NO: 37 - DNA, 20 bp, Artificial Sequence
  Synthetic gRNA target sequence for HDAC9sg1
agctttgatccaatgatgtg

SEQ ID NO: 38 - DNA, 20 bp, Artificial Sequence
  Synthetic gRNA target sequence for HDAC9sg2
aacagcatgagaacttgaca

SEQ ID NO: 39 - DNA, 20 bp, Artificial Sequence
  Synthetic gRNA target sequence for HDAC9sg3
tttccctctaaagtaacatg

SEQ ID NO: 40 - DNA, 20 bp, Artificial Sequence
  Synthetic gRNA target sequence for ICAM1sg1
cgaagccagaggtctcagaa

SEQ ID NO: 41 - DNA, 20 bp, Artificial Sequence
  Synthetic gRNA target sequence for ICAM1sg2
tgacgtgtgcagtaatactg

SEQ ID NO: 42 - DNA, 20 bp, Artificial Sequence
  Synthetic gRNA target sequence for ICAM1sg3
gcccgctgaggtcacgacca

SEQ ID NO: 43 - DNA, 20 bp, Artificial Sequence
  Synthetic gRNA target sequence for IL8 (CXCL8)sg1
atttctgtgttggcgcagtg

SEQ ID NO: 44 - DNA, 20 bp, Artificial Sequence
  Synthetic gRNA target sequence for IL8(CXCL8) sg2
cagagctgcagaaatcagga

SEQ ID NO: 45 - DNA, 20 bp, Artificial Sequence
  Synthetic gRNA target sequence for IL8(CXCL8) sg3
atttctgcagctctgtgtga

SEQ ID NO: 46 - DNA, 20 bp, Artificial Sequence
  Synthetic gRNA target sequence for ITGB7sg1
agagtgttcaagggtcacgg

SEQ ID NO: 47 - DNA, 20 bp, Artificial Sequence
  Synthetic gRNA target sequence for ITGB7sg2
ccacgtccgaatcaaccaga

SEQ ID NO: 48 - DNA, 20 bp, Artificial Sequence
  Synthetic gRNA target sequence for ITGB7sg3
ccgggtatccctcagcacga

SEQ ID NO: 49 - DNA, 20 bp, Artificial Sequence
  Synthetic gRNA target sequence for KCND3sg1
tctggtcatgaccaacaacg

SEQ ID NO: 50 - DNA, 20 bp, Artificial Sequence
  Synthetic gRNA target sequence for KCND3sg2
gtgcgtcatgatcttcaccg

SEQ ID NO: 51 - DNA, 20 bp, Artificial Sequence
  Synthetic gRNA target sequence for KCND3sg3
gacaatggtgtaccaaaacg

SEQ ID NO: 52 - DNA, 20 bp, Artificial Sequence
  Synthetic gRNA target sequence for KITsg1
gcctaatctcgtcgcccacg

SEQ ID NO: 53 - DNA, 20 bp, Artificial Sequence
  Synthetic gRNA target sequence for KITsg2
attacgaaaccaatcagcaa

SEQ ID NO: 54 - DNA, 20 bp, Artificial Sequence
  Synthetic gRNA target sequence for KITsg3
gaatggcatgctccaatgtg

SEQ ID NO: 55 - DNA, 20 bp, Artificial Sequence
  Synthetic gRNA target sequence for MAPK11sg1
cccctaccagacggagccgt

SEQ ID NO: 56 - DNA, 20 bp, Artificial Sequence
  Synthetic gRNA target sequence for MAPK11sg2
ggtggatgatcccggccgag

SEQ ID NO: 57 - DNA, 20 bp, Artificial Sequence
  Synthetic gRNA target sequence for MAPK11sg3
tgaccagctgaagcgcatca

SEQ ID NO: 58 - DNA, 20 bp, Artificial Sequence
  Synthetic gRNA target sequence for NPR1sg1
gatcgacgttagctccaacg

SEQ ID NO: 59 - DNA, 20 bp, Artificial Sequence
  Synthetic gRNA target sequence for NPR1sg2
gtgtattaggtatgactacg

SEQ ID NO: 60 - DNA, 20 bp, Artificial Sequence
  Synthetic gRNA target sequence for NPR1sg3
gctgtcggtgtaaaaaacca

SEQ ID NO: 61 - DNA, 20 bp, Artificial Sequence
  Synthetic gRNA target sequence for ODC1sg1
atcagagattgcctgcacga

SEQ ID NO: 62 - DNA, 20 bp, Artificial Sequence
  Synthetic gRNA target sequence for ODC1sg2
caacgctgggttgattacgc

SEQ ID NO: 63 - DNA, 20 bp, Artificial Sequence
  Synthetic gRNA target sequence for ODC1sg3
gaaggggctttacatgtgcg

SEQ ID NO: 64 - DNA, 20 bp, Artificial Sequence
  Synthetic gRNA target sequence for OXTRsg1
gcggctcaagaccgctgcag

SEQ ID NO: 65 - DNA, 20 bp, Artificial Sequence
  Synthetic gRNA target sequence for OXTRsg2
cctgcaagtacttgaccagg

SEQ ID NO: 66 - DNA, 20 bp, Artificial Sequence
  Synthetic gRNA target sequence for OXTRsg3
gctagctgtctacatcgtgc

SEQ ID NO: 67 - DNA, 20 bp, Artificial Sequence
  Synthetic gRNA target sequence for PDGFRAsg1
cgagacaggagtaccgtgga

SEQ ID NO: 68 - DNA, 20 bp, Artificial Sequence
  Synthetic gRNA target sequence for PDGFRAsg2
taagtcaggggaaacgattg

SEQ ID NO: 69 - DNA, 20 bp, Artificial Sequence
  Synthetic gRNA target sequence for PDGFRAsg3
ctggtcatttatagaaaccg

SEQ ID NO: 70 - DNA, 20 bp, Artificial Sequence
  Synthetic gRNA target sequence for PROCsg1
aattgctcgctggacaacgg

SEQ ID NO: 71 - DNA, 20 bp, Artificial Sequence
  Synthetic gRNA target sequence for PROCsg2
acgtgccaactccttcctgg

SEQ ID NO: 72 - DNA, 20 bp, Artificial Sequence
  Synthetic gRNA target sequence for PROCsg3
gccttctggtccaagcacgt

SEQ ID NO: 73 - DNA, 20 bp, Artificial Sequence
  Synthetic gRNA target sequence for PTGER4sg1
cagcgcgcaaaagagcacgt

SEQ ID NO: 74 - DNA, 20 bp, Artificial Sequence
  Synthetic gRNA target sequence for PTGER4sg2
gcccgcgtacatgtaggagt

SEQ ID NO: 75 - DNA, 20 bp, Artificial Sequence
  Synthetic gRNA target sequence for PTGER4sg3
gttcacagaagcaattcgga

SEQ ID NO: 76 - DNA, 20 bp, Artificial Sequence
  Synthetic gRNA target sequence for PTK7sg1
ctgcaacatcaagcacacgg

SEQ ID NO: 77 - DNA, 20 bp, Artificial Sequence
  Synthetic gRNA target sequence for PTK7sg2
gctctgaccatcagaaaggg

SEQ ID NO: 78 - DNA, 20 bp, Artificial Sequence
  Synthetic gRNA target sequence for PTK7sg3
gagcgtacgactgtgtacca

SEQ ID NO: 79 - DNA, 20 bp, Artificial Sequence
  Synthetic gRNA target sequence for SERPINE1sg1
agggtgagaaaaccacgttg

SEQ ID NO: 80 - DNA, 20 bp, Artificial Sequence
  Synthetic gRNA target sequence for SERPINE1sg2
cagacgcgatcttcgtccag

SEQ ID NO: 81 - DNA, 20 bp, Artificial Sequence
  Synthetic gRNA target sequence for SERPINE1sg3
gctgagttcaccacgcccga

SEQ ID NO: 82 - DNA, 20 bp, Artificial Sequence
  Synthetic gRNA target sequence for TNFsg1
ttggagtgatcggcccccag

SEQ ID NO: 83 - DNA, 20 bp, Artificial Sequence
  Synthetic gRNA target sequence for TNFsg2
agagctcttacctacaacat

SEQ ID NO: 84 - DNA, 20 bp, Artificial Sequence
  Synthetic gRNA target sequence for TNFsg3
ggagctgagagataaccagc

SEQ ID NO: 85 - DNA, 20 bp, Artificial Sequence
  Synthetic gRNA target sequence for TOP2Asg1
tgtacgcttatcctgactga

SEQ ID NO: 86 - DNA, 20 bp, Artificial Sequence
  Synthetic gRNA target sequence for TOP2Asg2
agcattgtaaagatgtatcg

SEQ ID NO: 87 - DNA, 20 bp, Artificial Sequence
  Synthetic gRNA target sequence for TOP2Asg3
tgaacaagtaaaccacaggt

SEQ ID NO: 88 - DNA, 20 bp, Artificial Sequence
  Synthetic gRNA target sequence for TRPV3sg1
tgtacgacatgatcctactg

SEQ ID NO: 89 - DNA, 20 bp, Artificial Sequence
  Synthetic gRNA target sequence for TRPV3sg2
aaccccaagtaccaacacga

SEQ ID NO: 90 - DNA, 20 bp, Artificial Sequence
  Synthetic gRNA target sequence for TRPV3sg3
aggatggactgcagatccga

SEQ ID NO: 91 - DNA, 20 bp, Artificial Sequence
  Synthetic gRNA target sequence for TUBA1Asg1
ctgtgataagttgctcaggg

SEQ ID NO: 92 - DNA, 20 bp, Artificial Sequence
  Synthetic gRNA target sequence for TUBA1Asg2
tgcgaattcggtccaacacg

SEQ ID NO: 93 - DNA, 20 bp, Artificial Sequence
  Synthetic gRNA target sequence for TUBA1Asg3
aagtctacaaacactgcccg

SEQ ID NO: 94 - DNA, 18 bp, Artificial Sequence
  Synthetic sequence for forward primer for first-round PCR of 298-bp fragment of Genomic DNA from cell pools infected with the sgRNA library
ggatccgcaacggaattc

SEQ ID NO: 95 - DNA, 20 bp, Artificial Sequence
  Synthetic sequence for reverseprimer for first-round PCR of 298-bp fragment of Genomic DNA from cell pools infected with the sgRNA library
ggttgcgtcagcaaacacag

SEQ ID NO: 96 - DNA, 78 bp, Artificial Sequence
  Synthetic sequence for forward primer for second-round PCR of 298-bp fragment of Genomic DNA from cell pools infected with the sgRNA library
caagcagaagacggcatacgagatgtgactggagttcagacgtgtgctcttccgatctggttgcgtcagcaaacacag

SEQ ID NO: 97 - DNA, 84 bp, Artificial Sequence
  Synthetic sequence for reverse primer for second-round PCR of 298-bp fragment of Genomic DNA from cell pools infected with the sgRNA library
  misc_feature (59)..(66): n is a, c, g, or t
aatgatacggcgaccaccgagatctacactctttccctacacgacgctcttccgatctnnnnnnnnggatccgcaacggaattc

SEQ ID NO: 98 - DNA, 20 bp, Artificial Sequence
  Synthetic sequence for forward primer for real-time PCR of anchor sequences
aatgatacggcgaccaccga

SEQ ID NO: 99 - DNA, 21 bp, Artificial Sequence
  Synthetic sequence for reverse primer for real-time PCR of anchor sequences
caagcagaagacggcatacga

SEQ ID NO: 100 - DNA, 20 bp, Artificial Sequence
  Synthetic gRNA target sequence for ATF6sg1
ttgtaggacaggtttagtca

SEQ ID NO: 101 - DNA, 20 bp, Artificial Sequence
  Synthetic gRNA target sequence for IRE1sg1
catgtttgacaaccgcgacg

SEQ ID NO: 102 - DNA, 20 bp, Artificial Sequence
  Synthetic gRNA target sequence for PERKsg1
ttatctaccatactacaaga

SEQ ID NO: 103 - DNA, 20 bp, Artificial Sequence
  Synthetic gRNA target sequence for PPP1R12Csg
gggtaaaccgactcccccga

SEQ ID NO: 104 - DNA, 20 bp, Artificial Sequence
  Synthetic gRNA target sequence for THUMPD3- AS1sg1
caggtaaaactgacgcacgg

SEQ ID NO: 105 - DNA, 20 bp, Artificial Sequence
  Synthetic gRNA target sequence for THUMPD3- AS1sg2
gggaccaccttatattccca

SEQ ID NO: 106 - DNA, 26 bp, Artificial Sequence
  Synthetic sequence for forward primer for ABCB1
aaattggcttgacaagttgtatatgg

SEQ ID NO: 107 - DNA, 21 bp, Artificial Sequence
  Synthetic sequence for reverse primer for ABCB1
caccagcatcatgagaggaag

SEQ ID NO: 108 - DNA, 24 bp, Artificial Sequence
  Synthetic sequence for forward primer for ABCC1
ctcctcctatagtggggacatcag

SEQ ID NO: 109 - DNA, 21 bp, Artificial Sequence
  Synthetic sequence for reverse primer for ABCC1
gtagtcccagtacacggaaag

SEQ ID NO: 110 - DNA, 21 bp, Artificial Sequence
  Synthetic sequence of forward primer for ABCC2
atgcagcctccataaccatga

SEQ ID NO: 111 - DNA, 24 bp, Artificial Sequence
  Synthetic sequence of reverse primer for ABCC2
cttcgtcttccttcaggctattca

SEQ ID NO: 112 - DNA, 23 bp, Artificial Sequence
  Synthetic sequence of forward primer for ABCG2
tcatcagcctcgatattccatct

SEQ ID NO: 113 - DNA, 21 bp, Artificial Sequence
  Synthetic sequence of reverse primer for ABCG2
ggcccgtggaacataagtctt

SEQ ID NO: 114 - DNA, 20 bp, Artificial Sequence
  Synthetic sequence of forward primer for NESTIN
ctgcgggctactgaaaagtt

SEQ ID NO: 115 - DNA, 20 bp, Artificial Sequence
  Synthetic sequence of reverse primer for NESTIN
aggctgagggacatcttgag

(19) United States
(12) Patent Application Publication
(10) Pub. No.: US 2023/0355596 A1
(43) Pub. Date: Nov. 9, 2023

(54) COMPOSITIONS AND METHODS OF USING NMDAR INHIBITORS AND KINASE INHIBITORS TO TREAT LIVER CANCER

(71) Applicants: The University of Hong Kong, Hong Kong (CN); Laboratory for Synthetic Chemistry and Chemical Biology Limited, Hong Kong (CN); Centre for Oncology and Immunology Limited, Hong Kong (CN)

(72) Inventors: Stephanie Kwai Yee Ma, Hong Kong (CN); Man Tong, Hong Kong (CN); Siu Lun Wong, Hong Kong (CN); Feng Xu, Hong Kong (CN)

(21) Appl. No.: 17/735,271

(22) Filed: May 3, 2022

(51) Int. Cl. A61K 31/445; A61K 45/06; A61P 35/00

(52) U.S. Cl. CPC A61K 31/445; A61K 45/06; A61P 35/00

(57) ABSTRACT

Methods and compositions for treating liver cancer in a subject in need thereof are provided. Pharmaceutical compositions including an effective amount of an NMDAR inhibitor in combination with a kinase inhibitor and methods of use thereof for treating cancer are disclosed. Administration of the combination of the active agents can be effective to reduce cancer cell proliferation or viability in a subject with cancer to the same degree, or a greater degree than administering to the subject the same amount of either active agent alone. The active agents can be administered together or separately. Methods of selecting and treating subjects with cancers, particular hepatocellular carcinoma are also provided.

### REFERENCE TO SEQUENCE LISTING

The Sequence Listing submitted May 3, 2022, as a text file named “UHK_01181_ST25.txt,” created on May 3, 2022, and having a size of 26,164 bytes is hereby incorporated by reference pursuant to 37 C.F.R. § 1.52(e)(5).

### FIELD OF THE INVENTION

The invention is generally directed to combination therapies including an NMDAR inhibitor and a kinase inhibitor for treating liver cancer.

### BACKGROUND OF THE INVENTION

Hepatocellular carcinoma (HCC) is the most common type of liver cancer with a very poor prognosis, ranking fourth as the most common cause of death among all cancers globally (Fitzmaurice et al. (2017) JAMA Oncol 3(4): pages 524-48). First-line treatment options for unresectable HCC include sorafenib, or lenvatinib, which target multiple kinases including Fms-related tyrosine kinase 4 (FLT4), fibroblast growth factor receptor (FGFR), and platelet-derived growth factor receptor (PDGFR) to inhibit tumor growth and angiogenesis (Yang et al. (2019) Nature Reviews 16(10): pages 589-604). However, these first-line treatments only extend overall survival of HCC patients for a few months, and the presence of drug-resistant cells poses a high chance of tumor relapse (Kudo et al (2018) Lancet, 391(10126): pages 1163-73; Llovet et al (2008) N Engl J Med, 359(4): pages 378-90). Thus, there remains a critical need to improve treatments for HCC.

Therefore, it is an object of the invention to provide compositions and methods of use thereof for treating hepatocellular carcinoma.

It is another object to provide compositions and methods for treating and/or preventing one or more of the pathological processes associated with development and progression of hepatocellular carcinoma.

### SUMMARY OF THE INVENTION

Compositions targeting an N-methyl-D-aspartate receptor (NMDAR) together with a kinase to treat and/or prevent the pathological processes associated with development and progression of hepatocellular carcinoma have been established.

Pharmaceutical compositions containing one or more NMDAR inhibitors and one or more kinase inhibitors and methods of making and using thereof are described herein. NMDAR inhibitors, such as ifenprodil, in combination with a kinase inhibitor, such as sorafenib, have shown a reduction in the viability and proliferation of cancer cells. The combination therapies can be used to improve the initial efficacy of one or the other of the active agents, or to re-sensitize cells that have become resistant to a dose (e.g., the maximum dose) of one or the other active agents when it is administered alone.

Pharmaceutical composition including an effective amount of the combination of an NMDAR inhibitor and a kinase inhibitor, or combinations thereof, and methods of use thereof for treating cancer are disclosed. Typically, administration of the combination of the two active agents (i.e., an NMDAR inhibitor and a kinase inhibitor) is effective to reduce cancer cell proliferation or viability in a subject with cancer to a greater degree than administering to the subject the same amount of the NMDAR inhibitor alone or the same amount of kinase inhibitor alone. In the most preferred embodiments, the reduction in cancer cell proliferation or viability in the subject with cancer is more than the additive reduction achieved by administering the NMDAR inhibitor alone or the kinase inhibitor alone. In some embodiments, in subjects with tumors, the combination is effective to reduce tumor burden, reduce tumor progression, or a combination thereof.

In the preferred embodiment, the NMDAR inhibitor is ifenprodil, or a prodrug, analog, or derivative, or pharmaceutically acceptable salt thereof. The dosage can be, for example, 1 mg-100 mg.

The kinase inhibitor is preferably a receptor tyrosine kinase inhibitor, and more preferably an inhibitor of Fibroblast Growth Factor Receptor or an inhibitor of Fms-related tyrosine kinase 4. Exemplary kinase inhibitors include sorafenib, lenvatinib, crizotinib, ceritinib, alectinib, brigatinib, bosutinib, dasatinib, imatinib, nilotinib, vemurafenib, dabrafenib, ibrutinib, palbociclib, ribociclib, cabozantinib, gefitinib, erlotinib, lapatinib, vandetanib, afatinib, osimertinib, ruxolitinib, tofacitinib, trametinib, axitinib, toceranib, nintedanib, pazopanib, regorafenib, sunitinib, dacomitinib, and ponatinib. Preferred kinase inhibitors include sorafenib and lenvatinib. The dosage of sorafenib can be 100 mg-1000 mg, preferably at or below the maximum tolerated dose in a human.

These compositions and methods are particularly effective for treating liver cancer including hepatocellular carcinoma. In some embodiments, the liver cancer is associated with aberrant Wnt/β-catenin signaling and/or Cdk2 signaling compared to non-cancerous cells.

Methods of treating subjects in need there using these combination therapies are also provided. The NMDAR inhibitor and the kinase inhibitor can be administered to the subject on the same day. In some embodiments, the two agents are administered simultaneously. The NMDAR inhibitor and the kinase inhibitor can be part of the same admixture or administered as separate compositions. In some embodiments, the separate compositions are administered through the same route of administration. In other embodiments, the separate compositions are administered through different routes of administration. For example, in some embodiments, an NMDAR inhibitor, such as ifenprodil, is intravenously through injection or infusion, and the kinase inhibitor such as sorafenib administered orally.

In some embodiments, the NMDAR inhibitor is administered to the subject prior to administration of the kinase inhibitor to the subject. The NMDAR inhibitor can be administered to the subject, for example, 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the kinase inhibitor to the subject.

In other embodiments, the kinase inhibitor is administered to the subject prior to administration of the NMDAR inhibitor to the subject. The kinase inhibitor can be administered to the subject, for example, 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the NMDAR inhibitor to the subject.

In some embodiments, the combination therapy includes administering to the subject one or more additional active agents. The second active agent can be a chemotherapeutic agent, for example, Gemcitabine, Oxaliplatin, Cisplatin, Doxorubicin, Capecitabine, and/or Mitoxantrone. In some embodiments, the disclosed methods also include surgery or radiation therapy.

Methods for characterizing the gene expression profile of cancer cells and/or the tumor microenvironment have also been developed to assess the extent to which the cancer cells or tumor associated cells are sensitive to treatment with NMDAR inhibitors in combination with kinase inhibitors. These methods are useful in the diagnosis, prognosis, selection of patients, and the treatment of cancer. For example, patients having cancer cells that express components of the Wnt/β-catenin signaling pathway and/or Cdk2 signaling pathway can be selected for treatment with the disclosed therapies.

In some embodiments, the cancer is characterized by down regulation of expression of one or more genes selected from the group consisting of Leucine Rich Repeat Containing G Protein-Coupled Receptor 5 (Lgr5), Axin 2 (Axin2), Hes Family BHLH Transcription Factor 1 (HES1), Alpha Fetoprotein (AFP), Nestin (NES), and Telomerase Reverse Transcriptase (Tert) following treatment. In other embodiments, the cancer is characterized by up-regulation of unfolded protein response effectors including phosphorylated IRE1-alpha and/or C/EBP Homologous Protein (CHOP) following treatment. In some embodiments, the combination therapy includes methods for selecting patients who would be amenable for NMDAR inhibitors and kinase inhibitors combination therapies, and for treating such patients. In some embodiments, the methods for selecting patients include the step of selecting a subject having a cancer characterized by overexpression of one or more genes involved in cancer stemness, Wnt/β-catenin signaling pathway and/or Cdk2 signaling pathway. As used herein, “characterized by” refers to the object being characterized as having or exhibiting the feature it is said to be characterized by.

In preferred embodiments, the NMDAR inhibitor and the kinase inhibitor are administered in an amount effective to reduce the serum concentration of one or more of Lens culinaris-reactive AFP, Golgi Protein 73, Asialo-alpha-acid glycoprotein, laminin, neopterin, and Glypican-3 compared to the serum concentration of one or more of Lens culinaris-reactive AFP, Golgi Protein 73, Asialo-alpha-acid glycoprotein, laminin, neopterin, and Glypican-3 prior to treatment.

Also disclosed are constructs and methods of using the constructs to identify combinatorial therapeutic targets for HCC. The constructs are typically a library of single guide RNAs (sgRNAs) directed to potential drug targets for suppressing HCC growth. In some embodiments, the sgRNAs comprise nucleotide sequences selected from the group consisting of SEQ ID NOs:1 to 93 and SEQ ID NOs:100-105. In some forms, the sgRNAs contain nucleotide sequences that are 80-100% identical to nucleotide sequence selected from the group consisting of SEQ ID NOs:1 to 93 and SEQ ID NOs:100-105. In some forms, the sgRNA contain nucleotide sequences that are 85-100% identical to nucleotide sequences selected from the group consisting of SEQ ID NOs:1 to 93 and SEQ ID NOs:100-105. In some forms, the sgRNAs contain nucleotide sequences that are 90-100% identical to nucleotide sequences selected from the group consisting of SEQ ID NOs:1 to 93 and SEQ ID NOs:100-105. In some forms, the sgRNAs contain nucleotide sequences that are 95-100% identical to nucleotide sequences selected from the group consisting of SEQ ID NOs:1 to 93 and SEQ ID NOs:100-105.

### BRIEF DESCRIPTION OF THE DRAWINGS

FIG. 1A is a schematic showing workflow of the combinatorial CRISPR screen. A barcoded pairwise single guide RNA (sgRNA) library was assembled using CombiGEM-CRISPR v2.0. MHCC97L-Cas9 cells were infected with the library via lentiviruses (on day −11), sorted for the infected cells (on day −3) and harvested on day 0 and day 7. Illumina HiSeq was applied to quantify the abundance of the barcodes. Screen hits were identified via comparison of barcode abundance between day 7 and day 0 cultured cell pool samples. FIGS. 1B-1D are graphs showing the distribution of barcode reads for the pairwise sgRNA combination within the plasmid and infected cell library pools. 98.7% and 96.6% of the expected sgRNA combinations were detected in the pooled plasmid library (FIG. 1C) and cell library (FIG. 1D), respectively. Most barcoded sgRNA combinations were detected within a 5-fold range from the mean barcode reads per combination (highlighted by the shaded areas). FIG. 1E is a volcano plot showing barcode abundance changes of each dual-gene (dark grey) and single-gene targeting sgRNA (light grey) combinations at day 7 versus day 0 post-infection. Multiple sgRNA combinations targeting the same pair of genes showed a mean log 2 fold change <−0.51 and a P value <0.05. They include FLT4+PDGFRA and FLT4+FGFR2 that encode kinases being the molecular targets of FDA-approved drugs sorafenib and lenvatinib, as well as NMDAR1+FLT4 and NMDAR1+FGFR3 that were identified as top hits for further validation. Data were collected from two biological replicates. FIG. 1F is a volcano plot illustrating the reproducibility for the normalized barcode representations between two biological replicates in MHCC97L-Cas9 cells infected with the pairwise sgRNA library. The horizontal dotted lines in the Bland-Altman plots indicate the 95% limits of agreement. FIG. 1G is a histogram showing the log 2 fold-change difference between two biological replicates for the pairwise sgRNA-infected cell pools. 96% of the pairwise sgRNA combinations had log₂ fold-change differences of <1 between the two replicates. FIGS. 1H and 1I are scatter plots showing the log₂ ratios of the normalized barcode counts for day 7 versus day 0 (FIG. 1H) and day 0 versus plasmid (FIG. 1I) samples. Only relatively minor changes in the representation of combinations were observed between day 0 and plasmid samples. The growth-inhibitory effects of sgRNA combinations that were confirmed with P<0.05 based on results obtained from two replicates (Table 3) are highlighted in dark grey, while combinations with control gRNA pairs are highlighted in black. Labeled sgRNA combinations were further validated in the study. FIGS. 1J and 1K are scatter dot plots showing the expression level of NMDAR1 detected in HCC tumor samples. Data was extracted from TCGA database. Number of non-tumor tissues and HCC tissues are 50 and 371, respectively (FIG. 1J). The 50 paired non-tumor and HCC tissues are plotted in the FIG. 1K. FIG. 1L is a survival curve showing that the expression of NMDAR1 was associated with poor overall survival of HCC patients. Data was extracted from TCGA database: 92 cases with high expression levels (top 25%) and 96 cases with low expression levels (bottom 25%).

FIGS. 2A-2L are graphs showing the individual validation of the pairwise combinations' growth inhibitory effects in HCC cells. MHCC97L-Cas9 cells were infected with vector control, and the indicated single and paired sgRNAs for 6 days. Cell viability was measured by MTT assay every 24 hours on the next 4 consecutive days (FIGS. 2A, 2D, 2G, and 2J). Data are mean±SD from biological replicates (n=4). Statistical significance was analyzed by one-way ANOVA with Tukey's post hoc test. Colony formation assay was performed on MHCC97L-Cas9 cells that were infected with vector control, and the indicated single and paired sgRNAs for 6 days and plated and cultured for 14 more days. The colony numbers (FIGS. 2B, 2E, 2H, and 2K) and colony areas (FIGS. 2C, 2F, 2I, and 2L) were quantified using ImageJ. Data shown are mean±SD from biological replicates (n=3). FIG. 2A is a line graph showing cell viability measured by absorbance at 570 nm and 650 nm (as a reference) by spectrophotometry at day 1, 2, 3, and 4, showing growth inhibitory effects on HCC cells transduced with vector control (▪), NMDAR1sg2 (▴), FLT4sg1 (▾), and the combination of NMDAR1sg2+FLT4sg1 (•). FIG. 2D is a line graph showing cell viability measured by absorbance at 570 nm and 650 nm (as a reference) by spectrophotometry at day 1, 2, 3, and 4, showing growth inhibitory effects on HCC cells transduced with vector control (▪), NMDAR1sg1 (▾), FLT4sg3 (▴), and the pairwise combination of NMDAR1sg1+FLT4sg3 (•). FIG. 2G is a line graph showing cell viability measured by absorbance at 570 nm and 650 nm (as a reference) by spectrophotometry at day 1, 2, 3, and 4, showing growth inhibitory effects on HCC cells transduced with vector control (▪), NMDAR1sg2 (▴), FGFR3sg1 (▾), and the pairwise combination of NMDAR1sg2+FGFR3sg1 (•). FIG. 2J is a line graph showing cell viability measured by absorbance at 570 nm and 650 nm (as a reference) by spectrophotometry at day 1, 2, 3, and 4, showing growth inhibitory effects on HCC cells transduced with vector control (▪), NMDAR1sg2 (▴), FGFR3sg2 (▾), and the pairwise combination of NMDAR1sg2+FGFR3sg2 (•). Data are mean±SD from biological replicates (n=4). Statistical significance was analyzed by one-way ANOVA with Tukey's post hoc test. In FIG. 2M, the MHCC97L-Cas9 cells were infected with vector control, and the indicated single and paired sgRNAs. The two safe harbor loci being simultaneously targeted were PPP1R12C and THUMPD3-AS1. The sgRNAs used in Safe Harbor Loci DKO 1 and 2 are listed in Table 2. Cell viability was measured by MTT assay 10 days post-infection and compared to the vector control sample. Data are mean±SD from biological replicates (n=4). Statistical significance was analyzed by one-way ANOVA with Tukey's post hoc test. FIGS. 2N-2Q are bar graphs showing results from the individual validation of the pairwise combinations' growth inhibitory effects in Hep3B cells using colony formation assay. Hep3B-Cas9 cells were infected with vector control, and the indicated single and paired sgRNAs for 6 days and plated and cultured for 14 more days. The colony numbers and areas were quantified (FIGS. 2N and 2P). Data shown are mean±SD from biological replicates (n=3). Statistical significance was analyzed by one-way ANOVA with Tukey's post hoc test. ****: P<0.0001; ***: P<0.001; **: P<0.01; *: P<0.05.

FIGS. 3A and 3B are line plots showing results that genetic ablation of NMDAR1+FLT4 and NMDAR1+FGFR3 suppressed the ability of HCC cells to form tumor spheres (log fraction non-responding). MHCC97L-Cas9 cells were infected with vector control, and the indicated single and paired sgRNAs for 6 days. Cells were then re-plated in spheroid growth medium at limiting dilutions for tumor sphere formation. The number of tumor spheres formed was counted after a 10-day culture. Stem cell frequency and 95% confidence intervals were calculated. Data were collected from three biological replicates. FIGS. 3C-3F are lines plots showing results from the infection of MHCC97L-Cas9 cells with a vector control, and the indicated paired sgRNAs for 6 days. Cells were then re-plated in spheroid growth medium at limiting dilutions for tumor sphere formation. The number of tumor spheres formed was counted after a 10-day culture. Stem cell frequency and 95% confidence intervals were calculated. Data were collected from three biological replicates. ****: P<0.0001; ***: P<0.001; **: P<0.01; *: P<0.05; n.s.: P>=0.05.

FIGS. 4A-4R are diagrams showing the cell viability from combined treatment of a NMDAR inhibitor (ifenprodil) and FLT4/FGFR inhibitor (infigratinib) in MHCC97L cells (FIGS. 4A-4C), combined treatment of a NMDAR inhibitor (ifenprodil) and FLT4/FGFR inhibitor (erdafitinib) in MCHC97L cells (FIGS. 4D-4F), combined treatment of a NMDAR inhibitor (ifenprodil) and FLT4/FGFR inhibitor (SAR131675) in MCHC97L cells (FIGS. 4G-4I), combined treatment of a NMDAR inhibitor (ifenprodil) and FLT4/FGFR inhibitor (sorafenib) in MCHC97L cells (FIGS. 4J-4L), combined treatment of a NMDAR inhibitor (ifenprodil) and FLT4/FGFR inhibitor (lenvatinib) in MCHC97L cells (FIGS. 4M-40), and a combined treatment of a NMDAR inhibitor (MK801) and FLT4/FGFR inhibitor (sorafenib) in MCHC97L cells (FIGS. 4P-4R). Cells were treated with the indicated drug pair at multiple doses for two days. Cell viability was measured by MTT assay. Synergy was determined based on the Bliss independence and HSA models for each drug pair. Dose combinations with a 95% lower confidence bound of the estimated excess over Bliss scores that is greater than 0 are in bold. Data are mean±SD from biological replicates (n=3).

FIGS. 5A-5D is a line plot showing the effects of sorafenib and/or ifenprodil in NMDAR1- and NMDAR2-ablated MHCC97L cells. FIG. 5A illustrates the cell viability (%) at 0, 2, 4, and 8 μM of sorafenib in MHCC97L-Cas9 cells that were infected with vector control (▪), or NMDAR1 (●), or NMDAR2Bsg (▴) RNAs. After 6-day post-infection, cells were treated with the indicated dose of sorafenib for 48 hours. Cell viability was determined by MTT assays. Data were collected from three biological replicates. Statistical significance was analyzed by one-way ANOVA with Tukey's post hoc test. #: P<0.05 and *: P<0.05 indicate comparisons between the vector control with NMDAR1sg1 and NMDAR2Bsg1, respectively. FIGS. 5B-5D are line graphs showing NMDAR1/2B-dependent ifenprodil-induced growth inhibition in sorafenib-treated cells. MHCC97L-Cas9 cells were infected with NMDAR1 and NMDAR2B sgRNAs. After 6-day post-infection, cells were treated with multiple doses of sorafenib together with ifenprodil for 48 hours. Cell viability was determined by MTT assays. Data were collected from three biological replicates. Statistical significance was analyzed by Student's t-test. ***: P<0.001; **: P<0.01; *: P<0.05; n.s.: P>=0.05. FIGS. 5E-5M are diagrams showing cell viability of combined treatment of a NMDAR inhibitor (ifenprodil) and a FLT4/FGFR inhibitor(s) (sorafenib) in Hep3B (FIGS. 5E-5G), Huh7 (FIGS. 5H-5J), and HepG2 (FIGS. 5K-5M) cells. Cells were treated with the indicated drug pair at multiple doses for two days. Cell viability was measured by MTT assay. Synergy was determined based on the Bliss independence and HSA models for each drug pair. Dose combinations with a 95% lower confidence bound of the estimated excess over Bliss scores that is greater than 0 are in bold. Data are mean±SD from biological replicates (n=3). FIGS. 5N-5V are diagrams showing that ifenprodil in combination with lenvatinib reduces viability and self-renewal ability of HCC cells. Cell viability of combined treatment of ifenprodil and lenvatinib in Hep3B (FIGS. 5N-5P), Huh7 (FIGS. 5Q-5S), and HepG2 (FIGS. 5T-5V) cells. Cells were treated with ifenprodil and lenvatinib at multiple doses for 2 days. Cell viability was measured by MTT assays. Data was collected from three biological replicates. Synergy was determined based on the Bliss independence and HSA models for each drug pair. Dose combinations with a 95% lower confidence bound of the estimated excess over Bliss score that is greater than 0 are in bold. Data are mean±SD from biological replicates. FIG. 5W is a line plot showing that ifenprodil in combination with lenvatinib suppressed the cell self-renewal ability of MHCC97L cells. Cells were pre-seeded in spheroid growth medium and treated with 20 μM ifenprodil+40 μM lenvatinib. The number of tumor spheres was counted after a 10-day culture. Stem cell frequency and 95% confidence intervals were calculated. Data were collected from three biological replicates. ****: P<0.0001; **: P<0.01; *: P<0.05; n.s.: P>=0.05.

FIGS. 6A-6L are diagrams showing the cell viability and self-renewal ability of L02 and HepaRG cells after co-treatment with ifenprodil and sorafenib/lenvatinib. Cell viability of combined treatment of ifenprodil and sorafenib (FIGS. 6A-6F) or lenvatinib (FIGS. 6G-6L) in L02 and HepaRG cells. Cells were treated with ifenprodil and sorafenib (FIGS. 6A-6F) or lenvatinib (FIGS. 6G-6L) at multiple doses for 2 days. Cell viability was measured by MTT assays. Data was collected from three biological replicates. Synergy was determined based on the Bliss independence and HSA models for each drug pair. Dose combinations with a 95% lower confidence bound of the estimated excess over Bliss score that is greater than 0 are in bold. Data are mean±SD from biological replicates. FIGS. 6M and 6N are line plots showing results from experiments in which L02 cells were pre-seeded in spheroid growth medium and treated with 10 μM ifenprodil+5 μM sorafenib (FIG. 6M) or 20 μM ifenprodil+40 μM lenvatinib (FIG. 6N). The number of tumor spheres was counted after a 10-day culture. Stem cell frequency and 95% confidence intervals were calculated. Data were collected from three biological replicates. P>0.05 was detected for all comparison groups. FIGS. 6O-6Q are diagrams showing the cell viability of sorafenib-resistant HepG2 cells after co-treatment with ifenprodil and sorafenib. In FIGS. 6O-6Q, sorafenib-resistant HepG2 cells were treated with ifenprodil and sorafenib at multiple doses for 2 days. Cell viability was measured by MTT assays. Data was collected from three biological replicates. Synergy was determined based on the Bliss independence and HSA models for each drug pair. Data are mean±SD from biological replicates.

FIG. 7A is a schematic showing the workflow of the limiting dilution spheroid formation assay. Cells pre-seeded in spheroid growth medium were treated with ifenprodil and sorafenib. The number of tumor spheres formed was counted after 10 days. FIGS. 7B-7E are line plots of the combined treatment of ifenprodil and sorafenib suppressed the ability of HCC cells to form tumor spheres. Limiting dilution spheroid formation assays were performed with MHCC97L cells treated with 10 μM ifenprodil+5 μM sorafenib (FIG. 7B), Hep3B cells treated with 5 μM ifenprodil+5 μM sorafenib (L), and Huh7 and HepG2 cells treated with 10 μM ifenprodil+10 μM sorafenib (FIGS. 7C-7E), for 10 days. Stem cell frequency and 95% confidence intervals were calculated. Data were collected from three biological replicates. FIGS. 7F-7J show results from the pre-treatment of ifenprodil and sorafenib reduces the self-renewal ability of HCC cells. FIG. 7F is a schematic depicting the workflow of the limiting dilution spheroid formation assay performed after ifenprodil and sorafenib pre-treatment. Cells were treated with ifenprodil and sorafenib for 48 hours, and then re-plated in spheroid growth medium without drug. The number of tumor spheres was counted after 10 days. FIGS. 7G-7J are line plots of results showing that pre-treatment of ifenprodil and sorafenib suppressed the self-renewal ability of HCC cells. Limiting dilution spheroid formation assays were performed with HCC cells treated with 10 μM ifenprodil+5 μM sorafenib (FIG. 7G), 5 μM ifenprodil+5 μM sorafenib (FIG. 7H), and 10 μM ifenprodil+10 μM sorafenib (FIGS. 71 and 7J), for 2 days. Stem cell frequency and 95% confidence intervals were calculated. Data were collected from three biological replicates. ****: P<0.0001; ***: P<0.001; **: P<0.01; *: P<0.05; n.s.: P>=0.05.

FIGS. 8A-8H are plots of results showing the expression changes in MHCC97L cells treated with ifenprodil and/or sorafenib. FIG. 8A is a bar graph of results showing that treatment of ifenoproil and/or sorafenib did not reduce expression of MDR transporters. MHCC97L cells were treated with 10 μM ifenprodil, 5 μM sorafenib, or combination of ifenprodil and sorafenib, for 24 hours. RNA was extracted from the drug(s)-treated cells for quantitative PCR. Fold change of each gene in drug(s)-treated group was normalized to DMSO-treated group. FIG. 8B is a heat map showing the log₂ fold-change values (Log 2(FC)) among differentially expressed genes (DEGs) identified by RNA-seq. Log 2(FC) of DEGs found in at least one treatment condition (IFEN: 10 μM ifenprodil, SOR: 5 μM sorafenib, and I+S: 10 μM ifenprodil and 5 μM sorafenib; treated for 24 hours) were compared to DMSO-treated control in MHCC97L cell line. DEGs are ordered from the lowest to the highest Log 2(FC) in each condition respectively. Data were collected from three biological replicates. FIG. 8C is a heatmap showing the log₂(FC) among significantly differentially expressed gene ontology groups identified in the RNA-seq data. Log 2(FC) of differentially expressed genes belonging to the Gene Ontology (GO) categories were found to be marked up-/down-regulated in the I+S treatment. Highlighted genes were subjected to further validations. FIGS. 8D-8G are bar graphs showing the gene expression changes revealed by RNA-seq in MHCC97L cells treated with ifenprodil and/or sorafenib. Drug treatment was performed as described in FIG. 8A. RNA was extracted from the drug(s)-treated cells for RNA-seq. Fold change of each gene in drug(s)-treated group was normalized to DMSO-treated group. Genes are grouped based on their involvement in ER stress-(FIG. 8D), WNT- and stem cell-(FIG. 8E), and cell cycle and DNA replication-(FIGS. 8F and 8G) related pathways FIG. 8H is a heatmap summary of the GO enrichment analysis. Median Log 2(FC) of differentially expressed genes (left panel), number of DEGs (middle panel), and the −Log 10 (false discovery rate) (right panel) of the GO categories among the top hits in GO enrichment analysis are shown. In FIG. 8H, the top hit child GO categories are collapsed into the parent GO and only the parent ones are plotted.

FIG. 9A is a bar graph showing results from the co-treatment of ifenprodil and sorafenib greatly increased the cellular level of ATF6. MHCC97L cells were transfected with a luciferase-based reporter construct harboring ATF6 binding sites. The luciferase signals were detected at day 2 post-transfection. Data are mean±SD from biological replicates (n=3). FIG. 9B is a bar graph showing results that the co-treatment of ifenoproil and sorafenib increases Annexin V-positive MHCC97L cells. Drug treatment was performed as described in FIG. 8A. Data are mean±SD from biological replicates (n=3). Statistical significance was analysed by one-way ANOVA with Tukey's post hoc test. ***: P<0.001; ***: P<0.001; n.s.: P>=0.05. FIG. 9C is a bar graph of results showing that co-treatment of ifenoproil and sorafenib reduces nestin mRNA expression. Drug treatment was performed as described in FIG. 8A. RNA was extracted from the drug(s)-treated cells for quantitative PCR. Fold change of each gene in drug(s)-treated group was normalized to DMSO-treated group. FIG. 9D is a bar graph of results showing that co-treatment of ifenprodil and sorafenib greatly increased the cellular level of ATF6. HepG2 cells were transfected with a luciferase-based reporter construct harboring ATF6 binding sites. The luciferase signals were detected at day 2 post-transfection. Data are mean±SD from biological replicates (n=3). Statistical significance was analyzed by one-way ANOVA with Tukey's post hoc test. FIG. 9E is a bar graph of results showing that co-treatment of ifenprodil and sorafenib reduced activity of WNT signaling. HepG2 cells were transfected with a TOPFlash (firefly luciferase-based) plasmid and a renilla-luciferase plasmid. Equal number of transfected cells were re-plated and treated with DMSO, 5 μM ifenprodil alone, 5 μM sorafenib alone, or combination of ifenprodil and sorafenib for 24 hours. Data are mean±SD from biological replicates (n=3). Statistical significance was analysed by one-way ANOVA with Tukey's post hoc test. ***: P<0.001; ***: P<0.001; **: P<0.01; *: P<0.05; n.s.: P>=0.05. FIGS. 9F and 9G are bar graphs of results showing that co-treatment of ifenprodil and sorafenib induced UPR-dependent G1-phase cell-cycle arrest. MHCC97L cells were treated with DMSO, 10 μM ifenprodil, 5 μM sorafenib, or combination of ifenprodil and sorafenib, for 24 hours. Cells were fixed and stained with PI and analyzed using flow cytometry. UPR-depleted MHCC97L cells were generated by infecting MHCC97L-Cas9 cells with three sgRNAs targeting IRE1-alpha, PERK, and ATF6. Data are mean±SD from biological replicates (n=3). FIG. 9H is a bar graph showing confirmation of reduced cellular level of ATF6 in UPR-depleted cells using a luciferase-based reporter construct harboring ATF6 binding sites. The luciferase signals were detected at day 2 post-transfection. Data are mean±SD from biological replicates (n=3). Statistical significance was analyzed by student's t test. ***: P<0.001. FIG. 9I is a bar graph of results showing that co-treatment of ifenprodil and sorafenib reduced activity of WNT signaling. MHCC97L cells were transfected with a TOPFlash (firefly luciferase-based) plasmid and a renilla-luciferase plasmid. Equal number of transfected cells were re-plated and treated with DMSO, 10 μM ifenprodil alone, 5 μM sorafenib alone, or combination of ifenprodil and sorafenib for 24 hours. Data are mean±SD from biological replicates (n=3). FIGS. 9J and 9K are line plots of results showing that depletion of UPR sensors impaired the inhibitory effects of the self-renewal ability induced by co-treatment of ifenprodil and sorafenib. Limiting dilution spheroid formation assays were performed with MHCC97L cells or UPR-depleted MHCC97L cells treated with 10 μM ifenprodil+5 μM sorafenib for 10 days. Stem cell frequency and 95% confidence intervals were calculated. Data were collected from three biological replicates.

FIGS. 10A-10R are diagrams of results showing that ifenprodil treatment enhances the efficacy of sorafenib in HCC patient-derived organoids. FIGS. 10A-10L are diagrams of results showing that combined treatment of ifenprodil and sorafenib synergistically inhibited the growth of multiple HCC patient-derived organoids. Dissociated cells of HCC-HK P1 (FIGS. 10A-10C), HCC #23 (FIGS. 10D-10F), HCC #10 (FIGS. 10G-10I), and HCC-HK P2 (FIGS. 10J-10L) organoids were seeded in complete culture medium. Three days after cell seeding when the cells formed small organoids, multiple doses of ifenprodil and sorafenib were added for additional 3 days. Cell viability was measured by Cell-Tier Glo assays after the treatment. Synergy was determined based on the Bliss independence and HSA models for each drug pair. Dose combinations with a 95% lower confidence bound of the estimated excess over Bliss score that is greater than 0 are in bold. Data are mean±SD from biological replicates (n=4). FIGS. 10M and 10N are bar graphs of results showing that ifenprodil in combination with sorafenib increases the level of UPR-related proteins (including phosphorylated IRE1-alpha and CHOP) and p21^{Cip1} and decreases the level of CDK2 and WNT signaling in HCC-HK P1 and HCC #23 organoids. HCC-HK P1 and HCC #23 organoids were treated with 20 μM ifenprodil and 8 μM sorafenib for 3 days. WNT signaling (lower panels) was measured using a lentiviral-based TOPFlash reporter. Data are mean±SD from biological replicates (n=4 for HCC-HK P1; n=3 for HCC #23). FIGS. 100-10R are line plots of results showing that ifenprodil in combination with sorafenib suppressed the cell self-renewal ability of HCC organoids. Limiting dilution spheroid formation assays were performed with the organoids treated with 20 μM ifenprodil and 8 μM sorafenib for 3 days. Stem cell frequency and 95% confidence intervals were calculated. Data were collected from three biological replicates.

FIGS. 11A-11I are graphs of results showing that treatment with ifenprodil in combination with sorafenib suppresses tumor formation in xenograft models. FIGS. 11A-11C are graphs of results showing that combined treatment of ifenprodil and sorafenib suppressed tumor growth of MHCC97L-derived xenograft in mice. MHCC97L cells were subcutaneously injected in nude mice and treated with 20 mg/kg ifenprodil, 28 mg/kg sorafenib, or combination of ifenprodil and sorafenib, for 21 days. Tumor size change (FIG. 11A), xenograft weight at endpoint (FIG. 11B), and body weight change during treatment (FIG. 11C) are shown (n=5 per treatment group). FIGS. 11D-11I are graphs of results showing that combined treatment of ifenprodil and sorafenib suppressed tumor growth of two patient-derived xenografts in mice. Patient-derived xenografts were subcutaneously injected in NOD/SCID mice and treated with 20 mg/kg ifenprodil, 28 mg/kg sorafenib, or combination of ifenprodil and sorafenib, for 21 days. Tumor size change (FIGS. 11D and 11G), xenograft weight at endpoint (FIGS. 11E and 11H), and body weight change during treatment (FIGS. 11F and 11I) are shown (n=14 for PDX1 and n=8 for PDX2 per group). FIGS. 11J-11L are growth curves of MHCC97L-derived and HCC patient-derived xenografts. Tumor size of each xenograft was measured every two days during the treatment period (n=5 per group for MHCC97L-derived xenograft (FIG. 11J), n=14 per group for HCC patient-derived xenograft PDX1, and n=8 per group for HCC patient-derived xenograft PDX2 (FIGS. 11K and 11L), respectively. Statistical significance was analyzed at end point by one-way ANOVA with Tukey's post hoc test. **: P<0.01; *: P<0.05.

FIGS. 12A-12C are graphs of results showing that combined treatment of ifenprodil and sorafenib in MHCC97L- and HCC patient-derived xenografts in vivo upregulates UPR and increases p21^{Cip1}. MHCC97L cells were subcutaneously injected in nude mice and treated with 20 mg/kg ifenprodil, 28 mg/kg sorafenib, or combination of ifenprodil and sorafenib, for 21 days. FIGS. 12A and 12B are bar charts representing the intensity of indicated signal from six random fields of immunohistochemical images of p-IRE1-alpha and p21^{Cip1} staining on tissues harvested from the resected MHCC97L-derived xenografts under a light microscope at 200× magnification. Statistical significance was analysed by one-way ANOVA with Tukey's post hoc test. FIG. 12C is a dot plot showing results from the treatment of ifenprodil and/or sorafenib inhibited tumor vasculature in HCC patient-derived xenograft (PDX1) in mice. The dot plot represents the number of CD31-positive vessels from three immunohistochemical images of CD31 staining on tissues harvested from the resected PDX1 tumors treated with DMSO, ifenprodil, sorafenib, or combination of ifenprodil and sorafenib. under a light microscope at 200× magnification. Same drug treatment regimen was used as in FIGS. 12A and 12B. Statistical significance was analyzed by one-way ANOVA with Tukey's post hoc test. ****: P<0.0001; ***: P<0.001; **: P<0.01; *: P<0.05; n.s.: P>=0.05. FIGS. 12D-12J are graphs of results from the treatment with ifenprodil in combination with sorafenib suppresses tumor-initiating cell frequency in xenograft models. FIGS. 12D-12G are bar graphs representing the intensity of indicated signal from five random fields of immunohistochemical images of Lgr5 and Axin2 staining on tissues harvested from the resected MHCC97L-derived (FIGS. 12D and 12E) and patient-derived (FIGS. 12F and 12G) xenografts treated with DMSO, ifenprodil, sorafenib, or combination of ifenprodil and sorafenib. FIGS. 12H and 121 are diagrams of results showing that ifenprodil in combination with sorafenib suppressed the cell self-renewal ability of xenografts. The xenograft residuals from MHCC97L-derived and patient-derived models were dissociated and seeded for limiting dilution spheroid formation assays. Stem cell frequency and 95% confidence intervals were calculated. FIG. 12K is a diagram showing that combined treatment of ifenprodil and sorafenib decreased the repropagation capability of HCC cells in vivo. Residual tumor cells were harvested from the drug(s)-treated patient-derived xenografts and 10,000, 5,000, 1,000, and 500 cells were transplanted into secondary NOD/SCID mouse recipients (n=6 per group). Average tumor latency and incidence were recorded, and tumor-initiating cell frequency was calculated.

FIGS. 13A-13D are graphs showing results from treatment with ifenprodil in combination with sorafenib does not inhibit the metastatic ability of HCC cells in vivo. FIGS. 13A and 13B are dot plots from the treatment of ifenprodil and/or sorafenib did not affect the metastases of mouse HCC cells to form tumors in the lung. RIL-175 cells were injected through tail vein in nude mice and treated with 20 mg/kg ifenprodil, 28 mg/kg sorafenib, or combination of ifenprodil and sorafenib, for 10 days. Quantification of numbers of lung metastasis nodules from images (FIG. 13B) and quantification of bioluminescence signal (FIG. 13A) detected from the mice, their ex vivo resected tumors at the lung, and tumor sizes are shown (n=5 per treatment group) are shown. n.s.: P>=0.05. FIGS. 13C and 13D are graphs of results showing that combined treatment of ifenprodil and sorafenib suppressed tumor growth of RIL-175-derived xenograft in mice. RIL-175 cells were orthotopically injected into the liver of mice and treated with 20 mg/kg ifenprodil, 28 mg/kg sorafenib, or combination of ifenprodil and sorafenib, for 18 days. Xenograft weight at endpoint (FIG. 13C) and body weight change (FIG. 13D) during treatment are shown (n=8 per treatment group). Statistical significance was analyzed by one-way ANOVA with Tukey's post hoc test. ****: P<0.0001; **: P<0.01; *: P<0.05.

FIG. 14A is a bar graph showing the relative protein expression (normalized with the expression of GAPDH) of p-IRE1α, IRE1α, CHOP, p-eIF2α/eIF2α, p21^{cip1} and CDK2 in Western blot in MHCC97L cells treated with 10 μM ifenprodil (IFEN), 5 μM sorafenib (SOR), or combination of ifenprodil and sorafenib (I+S), for 18 hours. FIG. 14B is a bar graph showing the relative protein expression (normalized with the expression of GAPDH) of p-IRE1α, IRE1α, CHOP, p-eIF2α/eIF2α, p21^{cip1} and CDK2 in Western blot in MHCC97L cells treated with 5 μM ifenprodil (IFEN), 5 μM sorafenib (SOR), or combination of ifenprodil and sorafenib (I+S), for 48 hours. FIG. 14C is a bar graph showing percentage of MHCC97L cells in GO/G1-phase, S phase, or G2/M phrase of cell cycle when treated with DMSO, 10 μM ifenprodil, 5 μM sorafenib, or combination of ifenprodil and sorafenib, for 24 hours. Cells were fixed and stained with PI and analyzed using flow cytometry. Data are mean±SD from biological replicates (n=3). Statistical significance was analyzed by one-way ANOVA with Tukey's post hoc test.

### DETAILED DESCRIPTION OF THE INVENTION

### I. Definitions

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The terms “single guide RNA” or “sgRNA” refer to the polynucleotide sequence comprising the guide sequence, tracr sequence and the tracr mate sequence. “Guide sequence” refers to the around 20 base pair (bp) sequence within the guide RNA that specifies the target site and may be used interchangeably with the terms “guide” or “spacer.”

The term “dosage regime” refers to drug administration regarding formulation, route of administration, drug dose, dosing interval and treatment duration.

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount which is able to treat one or more symptoms of hepatocellular carcinoma (HCC), reverse the progression of one or more symptoms of HCC, halt the progression of one or more symptoms of HCC, or prevent the occurrence of one or more symptoms of HCC in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salt” is art-recognized, and includes relatively non-toxic, inorganic, and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine;

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including lipocalin-2 antagonists may inhibit or reduce the activity and/or quantity of one or more lipocalin-2 protein or variants thereof by about 10%, 20%, 30%, 40%, 50%, 75%, 85%, 90%, 95%, or 99% from the activity and/or quantity of the same lipocalin-2 protein or variants thereof in subjects that did not receive or were not treated with the compositions. In some embodiments, the inhibition and reduction are compared according to the level of mRNAs, proteins, cells, tissues, and organs.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with HCC are mitigated or eliminated, including, but are not limited to, reducing and/or inhibiting rate of tumor cell proliferation/growth, increasing the quality of life of those suffering from the disease, decreasing the dose of other medications required to treat the disease, delaying the progression of the disease, and/or prolonging survival of individuals.

The term ‘treating HCC” or “HCC treatment” means reducing, inhibiting, or alleviating one or more symptoms related to HCC in a subject suffering from HCC.

### II. Compositions

The combination therapies include administration of an effective amount of at least two active agents, one being a N-methyl-D-aspartate receptor (NMDAR) inhibitor and the other being a kinase inhibitor, to a subject in need thereof. In preferred embodiments, the kinase is a receptor tyrosine kinase including FLT4 and FGFR.

A. Active Agents

### 1. N-Methyl-D-Aspartate Receptor (NMDAR) Inhibitors

The combination therapies include an N-methyl-D-aspartate receptor (NDMAR) antagonist.

N-methyl-D-aspartate (NMDA) binds selectively to the ionotropic glutamate receptor NDMAR, and not to other glutamate receptors. The NMDA receptor controls non-selective passage of cations across the cell membrane and is activated upon interaction with both glutamate and glycine (or D-serine). Opening and closing of the ion channel is primarily gated by ligand binding, and the current flow through the ion channel is voltage dependent. Activation of the receptor permits positively charged ions to flow through the cell membrane, which plays an important role in the control of synaptic plasticity and memory.

The complex structure of the NMDA receptor provides multiple sites for therapeutic inhibition, including competitive antagonists and non-competitive antagonists: competitive NMDA antagonists interact with the glutamate-binding site of the receptor to directly inhibit the activity of glutamate; non-competitive antagonists of NMDA receptors block the associated ion channels in a use-dependent manner. The glycine site and the polyamine site of the NMDA receptor are also susceptible to antagonism.

The NDMA antagonist can reduce or inhibit the function of the NDMA receptor, or reduce or inhibit binding of endogenous ligands including, but not limited to, glutamate and glycine, to the NDMA receptor, or a combination thereof.

In some embodiments, the NMDAR antagonists are competitive blockers of the glutamate-binding site of the receptor. In some embodiments, the NMDAR antagonists indirectly inhibit the glutamate-binding activity of NMDAR. In other embodiments, the NMDAR antagonists are uncompetitive or noncompetitive blockers of the channel pore of the NDMAR. In other embodiments, the NMDAR antagonists are antagonists of the glycine co-regulatory site of the NDMAR. In other embodiments, the NMDAR antagonists indirectly inhibit the glycine-binding activity of NDMAR. In other embodiments, the NMDAR antagonists are antagonists of the Dizocilpine site of the NDMAR.

In preferred embodiments, the NDMAR antagonist reduces or inhibits passage of cations across the cellular membrane. For example, the NDMAR antagonist can reduce or inhibit the number of cations within the cell.

In some embodiments, the NDMAR antagonist is a glycine site antagonist. Glycine site NDMAR antagonists include 4-Chlorokynurenine (AV-101), 7-Chlorokynurenic acid, Kynurenic acid, and Phenylalanine.

In some embodiments, the NDMAR antagonist is a glutamate site antagonist. Glutamate site antagonists include AP5, AP7, CGP-37849, Kaitocephalin, LY-235959, Midafotel, PEAQX, Perzinfotel, and Selfotel.

In some embodiments, the NDMAR antagonist is a dizocilpine or related site antagonist. Dizocilpine or related site antagonists include Delucemine (NPS-1506), Dextromethorphan (DXM), Dextrorphan (DXO), Dexanabinol, Diphenidine, Dizocilpine (MK-801), Esketamine, Ketamine, Lanicemine, Memantine, Methoxetamine, Phencyclidine (PCP), Tiletamine, and Amantadine.

In some embodiments, the mechanism or region of antagonist binding to the NDMAR is not to the glycine-binding site, or to the glutamate binding site, or to the dizocilpine-binding site. In some embodiments, the mechanism of the NDMAR antagonist activity has yet to be determined. NDMAR antagonists which bind to an unknown site of NDMAR include Diethyl ether, Eliprodil, Hodgkinsine, Ifenprodil, Nitrous oxide (N₂O), Psychotridine, Traxoprodil, Xenon, Atomoxetine, Dextropropoxyphene, Ethanol (alcohol), Guaifenesin, Huperzine A, Ibogaine, Ketobemidone, Methadone, Minocycline, and Tramadol.

In preferred embodiments, the NMDAR inhibitor is ifenprodil. The chemical structure of ifenprodil is shown below.

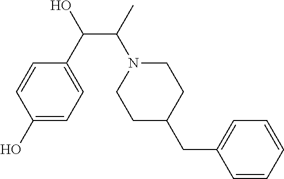 [SMILES: CC(C(O)c1ccc(O)cc1)N1CCC(Cc2ccccc2)CC1]

### 2. Kinase Inhibitors

The combination therapies include one or more kinase inhibitors. Multiple cellular kinases are involved in the development and progression of the HCC by promoting angiogenesis, cellular differentiation, proliferation, and survival.

Kinases are a class of enzyme that promote phosphorylation. Protein enzymes are chains of amino acids and when a phosphoryl group, i.e., PO32-, is covalently attached to one of the amino acids, it changes the three-dimensional configuration and function of the enzyme. Proteins are constantly being phosphorylated and dephosphorylated in living cells. Apoptosis, proliferation, and differentiation are all affected by phosphorylation. When these kinase enzymes go “wrong”, normal cellular function can go awry, and kinase deregulation can contribute to the growth of cancer cells. Drugs given to stop kinases can slow the proliferation of malignant cells and angiogenesis (growth of blood vessels). There are many kinases (538 in the human body) and many kinase inhibitor compounds have been identified. Some of these have proved to be useful in cancer treatment.

In some embodiments, the kinase inhibitor is a tyrosine kinase inhibitor (TKI). Most kinase inhibitors work on tyrosine kinases. Tyrosine kinases are important cellular signaling proteins that have a variety of biological activities, including cell proliferation and migration. Multiple kinases are involved in angiogenesis, including receptor tyrosine kinases such as the vascular endothelial growth factor receptor (VEGFR). Tyrosine kinase inhibitors (TKIs) are a class of chemotherapy medications that inhibit, or block, one or more of the enzyme tyrosine kinases. Cell membrane receptors are what scientists call molecular structures that send and receive signals from the environment. Some of the receptors are enzymes and catalyze biochemical reactions.

In some embodiments, the kinase inhibitor is an inhibitor of a Receptor Tyrosine Kinase (RTK). RTKs span the cell membrane with an intracellular (internal) and extracellular (external) portion. The intracellular portion removes a phosphate group, a process called de-phosphorylation, from the coenzyme messenger ATP. The extracellular portion has sites to which signal sending proteins and hormones can bind. Many of these signaling binders are growth factors.

In some embodiments, the kinase inhibitor is an inhibitor of a receptor for growth factors. Growth factors are involved in the initialization and regulation of cell cycles. The type of growth factor determines its effects on the cell. There are three primary growth factors that relate to tyrosine kinase. The receptors of these growth factors are members of the RTK family. Epidermal growth factors (EGF) help regulate cell growth and differentiation. Platelet-derived growth factors (PDGF) regulates cell growth and development. Vascular endothelial growth factors (VEGF) are involved in the creation of blood vessels.

Growth factors and kinases act together as though they are attached to an “on/off” switch. The removal of a phosphate group changes the shape and actions of the protein. This essentially “turns on” the cellular action (or actions). When the cellular action(s) is completed, the phosphate group is removed, and that protein is “turned off.” This “on/off” process can become disrupted, often by a mutated kinase, and actions can become unregulated. An unregulated RTK bound to EGF, for example, could lead to uncontrolled growth and division in the cell. The rapid cell growth could then lead to cancer. Mutations of RTKs often lead to oncogenes, which are genes that help turn a healthy cell into a cancerous cell.

Tyrosine kinase inhibitors treat cancer by correcting this deregulation. Imatinib, for example, blocks a kinase receptor from binding to ATP, preventing the phosphorylation that would benefit the cancerous cell and promote cell division. Gefinitib inhibits EGFRs, preventing that signal from being stuck “on” and creating uncontrolled proliferation. Over 30 TKI medications, including imatinib and gefinitib, have been approved by the Food and Drug Administration for use in humans. One TKI, Toceranib (Palladia), was approved for the treatment of cancer in dogs. The human medications may inhibit one or more tyrosine kinases. Erlotinib (Tarceva), like Gefitinib, inhibits EGFR. Lapatinib (Tykerb) is a dual inhibitor of EGFR and a subclass called Human EGFR type 2. EGFR is not the only growth factor targeted. Sunitinib (Sutent) is multi-targeted, inhibiting PDGFR and VEGF.

Other tyrosine kinase inhibitors are more specialized. Sorafenib (Nexavar) targets a complex pathway that would lead to a kinase signaling cascade. Nilotinib (Tasinga) inhibits the fusion protein bcr-abl and is typically prescribed when a patient has shown resistance to imatinib.

In some embodiments, the combination therapy includes one or more inhibitors targeting one or more of FLT4, FGFR, EGFR, ERBB2, VEGFRs, Kit, PDGFRs, ABL, SRC, mTOR, and combinations thereof. In some embodiments, the combination therapy includes one or more of Crizotinib, Ceritinib, Alectinib, Brigatinib, Bosutinib, Dasatinib, Imatinib, Nilotinib, Vemurafenib, Dabrafenib, Ibrutinib, Palbociclib, Sorafenib, Ribociclib, Cabozantinib, Gefitinib, Erlotinib, Lapatinib, Vandetanib, Afatinib, Osimertinib, Ruxolitinib, Tofacitinib, Trametinib, Axitinib, Toceranib, Lenvatinib, Nintedanib, Pazopanib, Regorafenib, Sunitinib, Dacomitinib, and Ponatinib.

In some embodiments, the combination therapy includes a kinase receptor antagonist that is a pan-kinase receptor inhibitor. In other embodiments, the combination therapy includes a kinase receptor antagonist that is a specific inhibitor of one or more kinases. In preferred embodiments, the combination therapy includes one or more inhibitors targeting Fms-Related Tyrosine Kinase 4 (FLT4) and/or Fibroblast Growth Factor Receptor (FGFR).

Sorafenib

In a preferred embodiment, the combination therapy includes the kinase inhibitor sorafenib. Sorafenib is an oral bi-aryl urea, which inhibits multiple cell surface and downstream kinases involved in tumor progression. Cell surface tyrosine kinases inhibited by Sorafenib include VEGF receptor-(VEGFR-) 1, VEGFR-2, VEGFR-3, platelet-derived growth factor receptor-(PDGFR-)β, RET, c-KIT, and FMS-like tyrosine kinase-3. Sorafenib also inhibits the Ras/MAPK pathway, which involves extracellular signal-regulated kinases and multiple intracellular serine/threonine kinases, including Raf-1 (C-Raf) and B-Raf (wild and mutant-types). Ras/MAPK pathway activation could be due to the mutational activation of Ras oncogene or over expression of surface tyrosine kinases. Overexpression of these kinases is important in HCC proliferation and angiogenesis (Wilhelm S, et al. (2006), Nat Rev Drug Discov. 5(10): pages 835-844; Wilhelm S M, et al. (2004), Cancer Res. 64(19): pages 7099-109). Two phase III randomized placebo-controlled trials, the SHARP trial, conducted mainly in America and Europe (Llovet J M, et al. (2008) N Engl J Med. 359(4): pages 378-390), and a similar trial conducted in Asia (Cheng A L, et al. (2009) Lancet Oncol. 10(1): pages 25-34) reported improved overall survival with sorafenib. In the SHARP trial, the median overall survival was 10.7 months with sorafenib and 7.9 months with placebo. In the Asian study, the median overall survival was 6.5 months with sorafenib and 4.2 months with placebo. Sorafenib was generally well tolerated; toxicities were mild to moderate in severity, predominantly including diarrhea, fatigue, and hand-foot skin reaction.

Accordingly, in some embodiments, the combination therapy includes an effective amount of sorafenib and an effective amount of one or more of NMDAR inhibitors for preventing, treating, or alleviating one or more symptoms of cancer, particularly liver cancer.

The chemical structure of sorafenib is shown below.

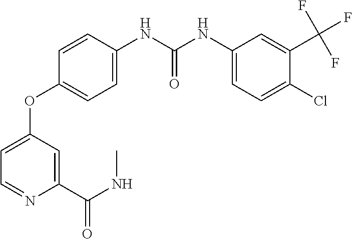 [SMILES: CNC(=O)c1cc(Oc2ccc(NC(=O)Nc3ccc(Cl)c(C(F)(F)F)c3)cc2)ccn1]

Lenvatinib

In a particular embodiment, the combination therapy includes the kinase inhibitor lenvatinib. Lenvatinib is a multireceptor tyrosine kinase inhibitor that inhibits the kinase activities of vascular endothelial growth factor (VEGF) receptors VEGFR1 (FLT1), VEGFR2 (KDR), and VEGFR3 (FLT4). Lenvatinib also inhibits other receptor tyrosine kinases that have been implicated in pathogenic angiogenesis, tumor growth, and cancer progression in addition to their normal cellular functions, including the fibroblast growth factor receptors FGFR1, 2, 3, and 4; and the platelet-derived growth factor receptor alpha, KIT, and RET.

In some embodiments, combination therapy includes an effective amount of lenvatinib and an effective amount of one or more of NMDAR inhibitors for preventing, treating, or alleviating one or more symptoms of cancer, particularly liver cancer.

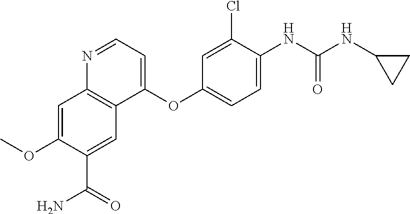 [SMILES: COc1cc2nccc(Oc3ccc(NC(=O)NC4CC4)c(Cl)c3)c2cc1C(N)=O]

Sunitinib

In a preferred embodiment, the combination therapy includes the kinase inhibitor sunitinib. Sunitinib is a multi-kinase blocker that targets VEGFR and PDGFR. Sunitinib was used in phase II clinical trials for HCC treatment, which led to an open-label phase III trial comparing it with sorafenib (Cheng A, et al. (2011), J Clin Oncol. 29 Suppl 15:4000). A total of 1073 patients were randomized to receive either sorafenib (544) or sunitinib (529). This trial was terminated early due to increased side effects and futility concerns.

Linifanib

In a particular embodiment, the kinase inhibitor is linifanib. Linifanib is a multi-kinase inhibitor targeting VEGFR and PDGFR along with other kinases. It was found to be effective in the treatment of the HCC with an acceptable safety profile in a single arm phase II clinical trial (Toh H C, et al. (2013), Cancer. 119: pages 380-387).

Brivanib

In a particular embodiment, the kinase inhibitor is brivanib. Brivanib is a selective inhibitor of fibroblastic growth factor receptor and VEGFR. It showed somewhat promising results in the phase II trials as first line (median overall survival: 10 months) and second line (median overall survival: 9.5 months) treatment agent for HCC (Park J W, et al. (2011) Clin Cancer Res. 17:1973-1983; Finn R S et al. (2012), Clin Cancer Res. 18: pages 2090-2098). Brivanib was tried in a phase III BRISK-PS trial as a secondary treatment agent (failed prior systemic treatment due to side effects or progression of the disease) for the treatment of HCC. The median length of overall survival was 9.4 months for brivanib recipients versus 8.2 months in the placebo group, which was not statistically significant (P=0.33) (Llovet J M, et al (2012). 47^{th} International Liver Congress (EASL 2012); Abstract 1398). Another phase III trial, BRISK-PS, compared brivanib with sorafenib as first line treatment agent for HCC (Johnson P J, et al. (2013) J Clin Oncol. 31: pages 3517-3524). Median survival was 9.5 months in the brivanib group compared with 9.9 months in the sorafenib group, which was not statistically significant. Sorafenib was better tolerated than brivanib leading to lesser discontinuation rate (33% vs 43% respectively).

Tivantinib

In a particular embodiment, the kinase inhibitor is tivantinib. Tivantinib is an oral MET receptor tyrosine kinase inhibitor. When added to sorafenib, it had synergistic effect against HCC as noted in a phase I clinical trial (Martell R E, et al. (2012) Proc Am Soc Clin Oncol. 30 (No. 15 Suppl): Abstract 4117). In a randomized, placebo-controlled, double-blind, phase II trial, tivantinib was used as a second line agent for the treatment of HCC in previously unresectable HCC who progressed or could not tolerate the first line systemic therapy (Santoro A, et al. (2013) Lancet Oncol. 14(1): pages 55-63). The patients were randomly assigned to receive tivantinib (n=71) or placebo (n=36). Time to progression of HCC was longer in tivantinib group (1.6 months) than the placebo group (1.4 months) (HR=0.64; P=0.04). The subgroup of patients who received tivantinib and expressed high tissue MET levels (n=22) had even longer median time to progression of HCC (2.7 months). A randomized, double-blinded, controlled phase III study (METIV-HCC trial) is currently underway to determine the efficacy and safety of tivantinib plus sorafenib compared to sorafenib alone in the patients with previously unresectable cancer as a first line treatment agent.

Everolimus

In a particular embodiment, the kinase inhibitor is everolimus. Everolimus is an inhibitor of mTOR. A phase I/II single arm trial using everolimus in advanced HCC patients (unresectable) with and without prior systemic therapy for HCC showed that the median progression free survival of 28 patients was 3.8 months (95% CI: 2.1-4.6) and overall survival was 8.4 months (95% CI: 3.9-21.1) (Zhu A X, et al. (2011) Cancer 117(22): pages 5094-5102). A randomized, double blind, placebo control phase III trial (EVOLVE-1) is underway to assess the role of everolimus in unresectable HCC patients who failed prior treatment with sorafenib.

Additional FLT4 and/or FGFR inhibitors include infigratinib, erdafitinib, and SAR131675.

### a. Fms-Related Tyrosine Kinase 4 (FLT4) Antagonists

In some forms the combination therapies include one or more NMDAR inhibitors with an Fms-related tyrosine kinase 4 (FLT4) antagonist. Fms-related tyrosine kinase 4, also known as FLT4, is a tyrosine kinase receptor for vascular endothelial growth factors C and D (VEGFC and VEGFD). FLT4 is thought to be involved in lymph angiogenesis, maintenance of the lymphatic endothelium and is central to the development of the vascular network and the cardiovascular system during embryonic development. FLT4 promotes proliferation, survival, and migration of endothelial cells, and regulates angiogenic sprouting. Signaling by activated FLT4 leads to enhanced production of VEGFC, and to a lesser degree VEGFA, thereby creating a positive feedback loop that enhances FLT4 signaling. Modulates KDR signaling by forming heterodimers. The secreted isoform 3 may function as a decoy receptor for VEGFC and/or VEGFD and play an important role as a negative regulator of VEGFC-mediated lymph angiogenesis and angiogenesis. Binding of vascular growth factors to isoform 1 or isoform 2 leads to the activation of several signaling cascades; isoform 2 seems to be less efficient in signal transduction, because it has a truncated C-terminus and therefore lacks several phosphorylation sites. Mediates activation of the MAPK1/ERK2, MAPK3/ERK1 signaling pathway, of MAPK8 and the JUN signaling pathway, and of the AKT1 signaling pathway.

The FLT4 antagonist can reduce or inhibit the function of the FLT4 gene, or the FLT4 gene product, or reduce or inhibit binding of endogenous ligands to the FLT4 receptor, or a combination thereof. In some embodiments, the FLT4 inhibitor is a molecule, which binds to the kinase domain of FLT4, or to one or more ectodomains of FLT4, or to the kinase domain of FLT4 and to the ectodomain of FLT4. In some embodiments, the FLT4 inhibitor is a molecule which prevents or reduces binding of VEGFC to FLT4, or prevents or reduces binding of VEGFD to FLT4, or prevents or reduces binding of both VEGFC and VEGFD to FLT4.

In some embodiments, the FLT4 inhibitor is MAZ51, SAR131675, AAL-993, Axitinib, linifanib, SU4312, sunitinib, VD6474 (vandetanib), vatalanib, motesanib, MK-2461, E3810 (lucitanib), MGCD-265, NVP-TAE226, AT9283, NVP-AEW541, or E7078 (lenvatinib).

### b. Fibroblast Growth Factor Receptor (FGFR) Inhibitors

In some forms, the combination therapies include one or more NMDAR inhibitors with one or more inhibitors targeting Fibroblast Growth Factor Receptor (FGFR). In particular embodiments, the combination therapy includes a Fibroblast Growth Factor Receptor 3 (FGFR3) inhibitor.

FGFRs are a family of membrane receptor tyrosine kinases that play central roles in developmental and adult cells. Dysregulation of FGFRs is implicated in a wide variety of cancers.

The human fibroblast growth factor receptor (FGFR) family includes four members: FGFR1, FGFR2, FGFR3, and FGFR4. The native ligand of FGFRs is fibroblast growth factors (FGFs). Through its extracellular domain, FGFR recognizes and is stimulated by specific FGFs. The FGF binding pocket is formed by the D2 and D3 subregions. The binding of FGFs drives the dimerization of FGFRs, which subsequently induces a trans-autophosphorylation event of the intracellular kinase domains, followed by the activation of downstream transduction pathways. Through triggering downstream signaling pathways, FGFRs participate in various vital physiological processes, such as proliferation, differentiation, cell migration and survival.

The FGFR antagonist can reduce or inhibit the function of the FGF receptor, or reduce or inhibit binding of endogenous ligands including, but not limited to, FGFs, to the FGF receptor, or a combination thereof. In some embodiments, the FGFR inhibitor is a molecule which binds to the kinase domain of FGFR, or to one or more ectodomains of FGFR, or to the kinase domain of FGFR and to the ectodomain of FGFR.

In some embodiments the FGFR antagonist is an inhibitor of FGFR. In some embodiments the FGFR antagonist is non-selective, multiple-kinase inhibitor. Non-selective FGFR inhibitors include ponatinib (AP24534) (O'Hare et al. (2009) Cancer Cell. 16(5): pages 401-412), dovitinib (Trudel et al. (2005) Blood 105(7): pages 2941-2948), lucitanib (Bello, et al. (2011) Cancer Res. 71(4): pages 1396-1405), and nintedanib (Hilberg et al. (2008) Cancer Res. 68(12): pages 4774-4782).

In some embodiments the FGFR inhibitor is a non-covalent pan-FGFR inhibitor, for example, a multiple-kinase inhibitor. Therefore, in some embodiments the FGFR inhibitor is a selective, pan-FGFR inhibitor. FGFR-selective inhibitors include AZD4547 (Gavine et al. (2012) Cancer Res. 72(8): pages 2045-2056) and LY2874455 (Zhao G et al. (2011) Mol. Cancer. Ther. 10(11): pages 2200-2210).

In some embodiments, the FGFR inhibitor is a molecule which covalently binds to and competitively blocks the FGF binding site of the receptor. In some embodiments, the FGFR inhibitor is a molecule which binds irreversibly to the FGFR. FGFR inhibitors which bind covalently to the receptor include BLU9931 (Hagel et al. (2015) Cancer Discov. 5(4): pages 424-437), FIIN-3 (Tan L et al. (2014) Proc. Natl. Acad. Sci. USA 111(45): pages E4869-E4877), FIIN-2 (Huang Z, et al. (2015) ACS Chem. Biol. 10(1): pages 299-309), CGA159527 (Fairhurst, et al. (2017) Med Chem. Comm. 8: pages 1604-1613), H3B-6527 (Ho H. K., et al. (2014) Drug Discov. 19: pages 51-62), and TAS-120 (Kalyukina, et al. (2019) Chem. Med. Chem. 14(4): pages 494-500), BLU-554 (Hagel, et al. (2015) Cancer Discov. 5(4): pages 424-437) and FGF401 (Hierro, et al. (2015), Semin. Oncol. 42(6): pages 801-819).

In some embodiments, the FGFR inhibitor is a molecule which binds to the ectodomain of FGFR. In some embodiments the FGFR inhibitor molecule is an antibody. In some embodiments the FGFR inhibitor molecule is a monoclonal antibody, such as FPA144 (Pierce, et al. (2014) J. Clin. Oncol. 32(15) suppl Abstract e15074), BAY 1187982 (Sommer, et al. (2016) Cancer Res. 76(21): pages 6331-6339), BAY 1179470 (Schatz, et al. (2014) Cancer Res. 74(19_Supplement): Abstract 4766), MFGR1877S (Trudel, et al. (2012) Blood. 120(21): page 4029), and FP-1039/GSK3052230 (Blackwell, et al. (2016) Oncotarget 7(26): pages 39861-39871).

In some embodiments, the FGFR inhibitor is a molecule which binds to the ectodomain of FGFR in a non-FGF competitive manner and inhibits FGF-induced signaling. In some embodiments, the FGFR inhibitor is a small molecule which allosterically binds to the ectodomain of FGFR, such as SSR128129E (Herbert, et al. (2013) Cancer Cell. 23(4): pages 489-501).

B. Formulations

Formulations of, and pharmaceutical compositions including one or more active agents are provided. The combination therapies can include administration of the active agents together in the same admixture, or in separate formulations. Therefore, the pharmaceutical compositions can include an NMDAR inhibitor and a kinase inhibitor, or combinations of one or more than one NMDAR inhibitor and one or more than one kinase inhibitor. In some embodiments, the pharmaceutical compositions can include one or more additional active agents. Therefore, in some embodiments, the pharmaceutical composition includes two, three, or more active agents. The pharmaceutical compositions can be formulated as a pharmaceutical dosage unit, referred to as a unit dosage form.

Formulations of combination therapies typically include an effective amount of an admixture of NMDAR inhibitor and a kinase inhibitor, or an effective amount of an admixture of more than one NMDAR inhibitor and more than one kinase inhibitor. Effective amounts of the combined active agents are discussed in more detail below. It will be appreciated that in some embodiments the effective amount of an NMDAR inhibitor in combination with a kinase inhibitor in a combination therapy is different from the amount that would be effective for the NMDAR inhibitor, or the kinase inhibitor to achieve the same result when administered individually. For example, in some embodiments the effective amount of an NMDAR inhibitor, or a kinase inhibitor, is a lower dosage of the NMDAR inhibitor, or kinase inhibitor in a combination therapy than the dosage of the NMDAR inhibitor, or kinase inhibitor that is effective when one of the agents is administered without the other. In other embodiments, the dosage of one agent is higher and the dosage of the other agent is lower than one agent is administered without the other. In some case, the agents are not effective when administered alone, and only effective when administered in combination.

### 1. Delivery Vehicles

The active agents can be administered and taken up into the cells of a subject with or without the aid of a delivery vehicle. Appropriate delivery vehicles for the disclosed active agents are known in the art and can be selected to suit the particular inhibitor. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

The active agent(s) can be incorporated into a delivery vehicle prepared from materials which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances, and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (monoglycerides, diglycerides and triglycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C. The release point and/or period of release can be varied as discussed above.

### 2. Pharmaceutical Compositions

Pharmaceutical compositions including active agent(s) with or without a delivery vehicle are provided. Pharmaceutical compositions can be for administration by parenteral (intramuscular, intraperitoneal, intravenous (IV) or subcutaneous injection), enteral, or transmucosal (nasal, vaginal, rectal, or sublingual) routes of administration or using bio erodible inserts and can be formulated in dosage forms appropriate for each route of administration.

In certain embodiments, the compositions are administered locally, for example, by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the compositions which is greater than that which can be achieved by systemic administration. Targeting of the molecules or formulation can be used to achieve more selective delivery.

### a. Formulations for Parenteral Administration

Active agent(s) and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

### b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment, and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and ® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art, such as direct compression, wet granulation, or dry granulation processes. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bio erodible, gradually hydrolysable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxy methylcellulose, hydroxypropyl methylcellulose, hydroxypropyl methylcellulose acetate succinate, hydroxypropyl methylcellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinyl acetate phthalate, vinyl acetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 weight % to 100 weight % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e., non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

C. Adjunct and Additional Therapies and Procedures

The combination therapies can be administered to a subject in combination with one or more adjunct therapies or procedures, or can be an adjunct therapy to one or more primary therapies or producers. The additional therapy or procedure can be simultaneous or sequential with the combination therapy. In some embodiment the additional therapy is performed between drug cycles or during a drug holiday that is part of the combination therapy dosage regime. In preferred embodiment, the additional therapy is a conventional treatment for cancer, more preferably a conventional treatment for liver cancer. For example, in some embodiments, the additional therapy or procedure is surgery, transplant surgery, a radiation therapy, or chemotherapy. For example, in a particular embodiment, combination therapies used simultaneously or sequentially with a regime of a chemotherapeutic agent, e.g., Gemcitabine (Gemzar), Oxaliplatin (Eloxatin), Cisplatin, Doxorubicin (pegylated liposomal doxorubicin), Capecitabine (Xeloda), Mitoxantrone (Novantrone), docetaxel or cabazitaxel. As discussed in more detail below, in some embodiment, the adjunct or additional therapy is part of the combination therapy.

### III. Methods of Treatment

It has been established that N-methyl-D-aspartate receptor (NMDAR) inhibitors can be used in combination with inhibitors of one or more kinases to provide enhanced antitumor activity as compared to the use of either agent alone. In preferred embodiments, the kinase is a receptor tyrosine kinase including FLT4 and FGFR.

Methods of treating one or more symptoms of liver cancer in a subject are provided. In certain embodiments, the methods include administering to a subject with cancer an effective amount of an NMDAR inhibitor, or a derivative, analog or prodrug, or a pharmacologically active salt thereof in combination with one or more inhibitors of a kinase, preferably receptor tyrosine kinase, to reduce or inhibit one or more symptoms of the cancer. In preferred embodiments, the NMDAR inhibitor and inhibitors of a kinase, preferably a receptor tyrosine kinase, can be used in combination to provide enhanced antitumor activity as compared to the use of either agent alone. The methods can include contacting one or more cancer cells expressing the NMDAR and kinase with an effective amount of an a NMDAR inhibitor in combination with one or more inhibitors of a kinase, preferably receptor tyrosine kinase, to decrease or inhibit the proliferation and/or viability of the cancer cells compared to untreated control cancer cells.

Aberrant Wnt/β-catenin signaling has been shown to be common in HCC tumors and to have significant clinical impact on tumor behavior, prognosis, and response to treatment. In certain embodiments the methods are more effective in treating cancer in a subject having cancer cells that exhibit aberrant expression of one or more genes involved in Wnt/0-Catenin signaling pathway. In preferred embodiments, the cancer cells exhibit increased Wnt/β-Catenin signaling pathway relative to non-cancer cells. In other embodiments, the methods are more effective in treating cancer in a subject having cancer cells that exhibit reduction in expression of one or more genes involved in Wnt/β-Catenin signaling pathway in response to combination treatment with an NMDAR inhibitor plus a kinase inhibitor. In certain embodiments the methods are more effective in treating cancer in a subject having cancer cells that exhibit expression of one or more genes involved in Cdk2 signaling pathway. In preferred embodiments, the cancer cells exhibit increased Cdk2 signaling pathway relative to non-cancer cells. In other embodiments, the methods are more effective in treating cancer in a subject having cancer cells that exhibit reduction in expression of one or more genes involved in Cdk2 signaling pathway in response to combination treatment with an NMDAR inhibitor plus a kinase inhibitor.

In some embodiments, the methods administer an effective amount of an NMDAR inhibitor and kinase inhibitor to activate or enhance UPR effectors, including phosphorylated IRE1-alpha and/or C/EBP Homologous Protein (CHOP) in the target cells; to increase the expression of p21^{ciP1}, a regulator of cell-cycle progression at G1/S phase; to downregulate CDK2 level; and/or to downregulate WNT signaling in cancer cells. In some embodiments, the methods administer an effective amount to reduce gene expression in one or more of Lgr5, Axin2, HES1, AFP, NES (Nestin), and Tert in cancer cells.

The NMDAR inhibitor and kinase inhibitor can be administered locally or systemically to the subject, or coated or incorporated onto, or into a device. Therefore, in some embodiments, an NMDAR inhibitor is administered locally or systemically to the subject, at the same time, or before, or after a kinase inhibitor is administered locally or systemically to the same subject.

Compositions for use in the treatment of the disclosed diseases are also provided. For example, a composition including an NMDAR inhibitor for use in a method of treating a subject with cancer, wherein the subject is one whom a composition including a kinase inhibitor has previously been or is concurrently being administered and wherein the response achieved following the administration of NMDAR inhibitor is greater than the response achieved by administering either the NMDAR inhibitor alone or the kinase inhibitor alone are disclosed.

In another embodiment, a composition including a kinase inhibitor for use in a method of treating a subject with cancer, wherein the subject is one whom a composition including an NMDAR inhibitor has previously been or is currently being administered and wherein the response achieved following the administration of the kinase inhibitor is greater than the response achieved by administering either the NMDAR inhibitor alone or the kinase inhibitor alone is provide. Suitable compositions, cancers to be treated, dosage regimes, and responses achieved by administering the combinations are discussed at length above. In particular embodiments the subject may have been previously administered one or more of the drugs, but not in combination.

Furthermore, it will be appreciated as discussed above, that the cancer may have developed a resistance to the previously administered active agent the active agent is administered in the absence of the combination. Therefore, in some embodiments, the subject population being treatment is defined as one in which the cancer being treated is resistant or insensitive to one or the other of the active agent when administered alone.

A. Methods For Selecting Patients For NMDAR Inhibitor and Kinase Inhibitor Combination Therapies

Methods for characterizing tumors and/or for characterizing the tumor microenvironment are provided. In particular embodiments, the methods characterize tumors so as to assess the extent to which the tumor cells and/or tumor infiltrating cells or tumor associated cells express genes associated with sensitivity to combination therapy with one or more NMDAR inhibitors in combination with one or more inhibitors of a kinase, preferably a receptor tyrosine kinase. For example, tumor cells and/or tumor infiltrating cells or tumor associated cells that are sensitive to more than additive effects of combination therapy with NMDAR inhibitor in combination with one or more inhibitors of a receptor tyrosine kinase can express genes involved in the Wnt/β-Catenin signaling pathway prior to treatment, and/or show up-regulation of UPR effectors, including phosphorylated IRE1-alpha and/or C/EBP Homologous Protein (CHOP) after combination treatment. In some embodiments, the methods diagnose and treat cancer and other diseases. Therefore, in some embodiments, the methods characterize a cell of a tumor. In particular embodiments, the methods include determining whether a cell of the tumor expresses one or more of the components of genes and signaling pathways involved in cancer sternness (including Nestin and Tert), specifically genes involved in the Wnt/β-Catenin signaling pathway including Lgr5 and Axin2.

Methods for assessing the amenability of subject to a proposed anti-cancer therapy are also provided. In some embodiments the methods include characterizing cells of a tumor of the subject by determining whether the cells of the tumor express genes that are associated with sensitivity to more than additive effects of NMDAR inhibitor in combination with one or more inhibitors of a receptor tyrosine kinase. For example, in some embodiments, subjects having cancer cells that express genes involved in the Wnt/β-Catenin signaling pathway are selected for treatment with an NMDAR inhibitor in combination with one or more inhibitors of a receptor tyrosine kinase. Methods for assessing the efficacy of an anti-cancer therapy provided to a subject are also disclosed. In some embodiments the methods include characterizing cells of a tumor of the patient during the course of the therapy or after the completion thereof, wherein said characterization can include determining whether the cells of the tumor express genes involved in the Wnt/β-Catenin signaling pathway, or show increased expression of UPR effectors, including phosphorylated IRE1-alpha and/or C/EBP Homologous Protein (CHOP) in response to the treatment.

Methods for selecting patients for anti-cancer therapy based on characterization of the tumor or tumor microenvironment are also provided. In some embodiments, cancer patient tumor samples are characterized prior to and following treatment with specific chemotherapeutic and/or biologic therapies, and/or other therapeutic interventions (e.g. radiation, cryoablation, surgical resection of the tumor, etc.), in order to see if the expression patterns of one or more of the components of genes and signaling pathways involved in cancer stemness (including Nestin and Tert), specifically genes involved in the Wnt/β-Catenin signaling pathway including Lgr5 and Axin2, within the tumor microenvironment have changed.

In some embodiments, the methods include detecting genes involved in the signaling pathways involved in cancer stemness (including Nestin and Tert), specifically genes involved in the Wnt/β-Catenin signaling pathway including Lgr5 and Axin2, alone, or in combination with one or more other biomarkers of cancer cells. Suitable methods of detection are known in the art. For example, in specific embodiments, the methods include a step of contacting the cell of the tumor with a molecule that immuno-specifically or physio-specifically binds proteins involved in the Wnt/β-Catenin signaling pathway, or examining RNA expression for genes involved in cancer stemness using qPCR, microarray methods, or RNA-Sequencing. In some preferred embodiments, the molecule that immuno specifically or physio specifically binds proteins involved in cancer stemness (including Nestin and Tert), and/or genes involved in the Wnt/β-Catenin signaling pathway. In particular embodiments, the genes involved in the Wnt/β-Catenin signaling pathway are Lgr5 and Axin2. In some embodiments, the molecule that immuno specifically or physio specifically binds proteins involved in cancer stemness molecule is an antibody or an antigen-binding fragment thereof. In a preferred embodiment, the antibody or antigen-binding fragment thereof is specific for the Lgr5 or Axin2 gene products.

B. Methods of Administration and Dosage Regimes

The combination therapies and treatment regimens typically include treatment of a disease or symptom thereof, or a method for achieving a desired physiological change, including administering to an animal, such as a mammal, especially a human being, an effective amount of an NMDAR inhibitor in combination with a kinase inhibitor to treat the disease or symptom thereof, or to produce the physiological change, wherein the chemical agents or components are administered together, such as part of the same composition, or administered separately and independently at the same time or at different times (i.e., administration of the NMDAR inhibitor and the kinase inhibitor is separated by a finite period of time from each other). Therefore, the term “combination” or “combined” is used to refer to either concomitant, simultaneous, or sequential administration of the NMDAR inhibitor and the kinase inhibitor. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

When used for treating cancer, the amount of NMDAR inhibitor present in a pharmaceutical dosage unit, or otherwise administered to a subject can be the amount effective to reduce the proliferation, viability, or a combination thereof of the cancer cells when administered in combination with a kinase inhibitor. Likewise, the amount of kinase inhibitor present in a pharmaceutical dosage unit, or otherwise administered to a subject can be the amount effective to reduce the proliferation, viability, or a combination thereof of the cancer cells when administered in combination with an NMDAR inhibitor. Therefore, in some embodiments the amount of the active agents is effective to decrease or inhibit the proliferation and/or viability of the cancer cells compared to untreated control cancer cells. In some embodiments, the amount of the active agents is effective to reduce, slow or halt tumor progression, or to reduce tumor burden in one or more tumors in the recipient, or a combination thereof. In some embodiment, the amount of the active agents is effective to alter a measurable biochemical or physiological marker. For example, in the case of liver cancer, the amount of the active agents can be effective to reduce one or more of the level of Lens culinaris-reactive AFP (also known as AFP-L3), Golgi Protein 73 (GP73), Asialo-alpha-acid glycoprotein (As AGP), laminin, neopterin, and/or Glypican-3 (GPC3) concentration in the blood compared to the AFP-L3, GP73, As AGP, laminin, neopterin, and/or GPC3 concentration prior to treatment. In some embodiments, the active agents are administered in an effective amount to reduce or prevent cancer progression, despite a rise in the level or amount of one or more of AFP-L3, GP73, As AGP, laminin, neopterin, and GPC3 concentration in the recipient.

In preferred embodiments, administration of the NMDAR inhibitor and the kinase inhibitor achieves a result greater than when the NMDAR inhibitor and the kinase inhibitor are administered alone or in isolation. For example, in some embodiments, the result achieved by the combination is partially or completely additive of the results achieved by the individual components alone. In the most preferred embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

The effect of the combination therapy, or individual agents thereof can depend on the disease or condition to be treated or progression thereof. For example, as illustrated in the Examples below, an agent such as sorafenib can be used a first or second line therapy for treatment of liver cancer. However, over time, the cancer can develop a resistance to sorafenib. Subsequent treatment of the cancer with sorafenib in combination with a NMDAR inhibitor such as ifenprodil “re-sensitizes” the cancer to sorafenib treatment. Accordingly, in some embodiments, the effect of the combination on a cancer can compared to the effect of the individual agents alone on the cancer.

A treatment regimen of the combination therapy can include one or multiple administrations of NMDAR inhibitor, and one or multiple administrations of kinase inhibitor. In certain embodiments, an NMDAR inhibitor can be administered simultaneously with a kinase inhibitor. Where an NMDAR inhibitor and a kinase inhibitor are administered at the same time, the NMDAR inhibitor and the kinase inhibitor can be in the same pharmaceutical composition.

In some embodiments, an NMDAR inhibitor and a kinase inhibitor are administered sequentially, for example, in two or more different pharmaceutical compositions. In certain embodiments, the NMDAR inhibitor is administered prior to the first administration of the kinase inhibitor. In other embodiments, the kinase inhibitor is administered prior to the first administration of the NMDAR inhibitor. For example, the NMDAR inhibitor and the kinase inhibitor can be administered to a subject on the same day. Alternatively, the NMDAR inhibitor and the kinase inhibitor are administered to the subject on different days.

In some embodiments, the kinase inhibitor is administered at least 1, 2, 3, 4, 5, 6, 7, 8, 9, 10, 11, 12, 13, 14, 15, 16, 17, 18, 19, 20, 21, 22, 23, 24 or 30 hours or days prior to or after administering the NMDAR inhibitor. In other embodiments, the NMDAR inhibitor is administered at least 1, 2, 3, 4, 5, 6, 7, 8, 9, 10, 11, 12, 13, 14, 15, 16, 17, 18, 19, 20, 21, 22, 23, 24 or 30 hours or days prior to or after administering of the kinase inhibitor. In certain embodiments, additive or more than additive effects of the administration of NMDAR inhibitor in combination with one or more kinase inhibitors is evident after one day, two days, three days, four days, five days, six days, one week, or more than one week following administration.

Dosage regimens or cycles of the agents can be completely or partially overlapping, or can be sequential. For example, in some embodiments, all such administration(s) of the NMDAR inhibitor occur before or after administration of the kinase inhibitor. Alternatively, administration of one or more doses of the NMDAR inhibitor can be temporally staggered with the administration of kinase inhibitor to form a uniform or non-uniform course of treatment whereby one or more doses of NMDAR inhibitor are administered, followed by one or more doses of kinase inhibitor, followed by one or more doses of NMDAR inhibitor; or one or more doses of kinase inhibitor are administered, followed by one or more doses of NMDAR inhibitor, followed by one or more doses of kinase inhibitor; etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

C. Diseases to be Treated

The combination therapies are typically used to treat cancer, preferably hepatocellular carcinoma. In some embodiments the methods include administering an effective amount of a kinase inhibitor in combination with an NDMAR inhibitor to treat or prevent liver cancer in a subject in need thereof. Other suitable cancers to be treated include advanced renal cell carcinoma, thyroid cancer, and FLT3-ITD positive acute myeloid leukemia, for which Cdk2 and/or WNT signaling have been associated with tumorigenesis and recurrence.

Suppression of the cancer's ability to grow can be measured using a biochemical assay, for example, measuring a decline in the AFP-L3, GP73, As AGP, laminin, neopterin, and/or GPC3 concentration in the blood, or by a morphometric analysis, for example by computerized tomography (CT), magnetic resonance imaging (MRI) or ultrasound. Therefore, in some embodiments the methods include the step of measuring one or more of AFP-L3, GP73, As AGP, laminin, neopterin, and/or GPC3 concentration in the blood of the recipient. The measurements can be made before and after administration of a kinase inhibitor in combination with an NDMAR inhibitor.

A cancer that has reacquired an ability to grow can be an increase in tumor growth, the emergence, reemergence, or aggravation of other symptoms such as ostealgia, or new sites of metastasis.

### 1. Hepatocellular Carcinoma

In some embodiments, methods of administering a kinase inhibitor in combination with an NDMAR inhibitor are effective for treatment of hepatocellular carcinoma (HCC). HCC is the most common primary liver malignancy and is a leading cause of cancer-related death worldwide.

Chronic liver disease and cirrhosis remain the most important risk factors for the development of HCC of which viral hepatitis and excessive alcohol intake are the leading risk factors worldwide.

Chronic viral hepatitis can lead to cirrhosis and/or HCC. Hepatitis B and C are the most common causes of chronic hepatitis in the world. Hepatitis B virus (HBV) is a double-stranded, circular DNA molecule with eight genotypes (A to H). Genotypes A and D are more common in Europe and the Middle East, while genotypes B and C are more common in Asia. Hepatitis B is transmitted via contaminated blood transfusions, intravenous injections, and sexual contact. Vertical transmission from mother to fetus is the leading cause for HBV infection worldwide. Five percent of the world's population is infected with Hepatitis B.

Several epidemiological studies have demonstrated significant hepatocarcinogenicity with chronic HBV infection. Hepatitis B carriers have a 10%-25% lifetime risk of developing HCC. Unlike other causes of chronic hepatitis, HBV is unique in that HCC can develop without evidence of cirrhosis. Genotype C has been associated with a higher risk of HCC than genotypes A, B, and D. Active infection with HBV carries an independent risk of HCC with HBV DNA levels >105/mL viral copies associated with a 2.5-3 times increased risk of developing HCC in 8-10 years follow-up. Hepatitis B surface antigen (HBsAg) is not the only hematological marker that carries a significant risk for development of HCC. Patients with positive hepatitis B core antibody (anti-HBc) who are HBs Ag-negative also remain at risk for development of HCC. The hepatocarcinogenicity of HBV can be significantly reduced with antiviral treatment for hepatitis B. Suppression of the virus can result in a significant 5-year reduction of the incidence of HCC from 13.7% (controls) to 3.7%, with the greatest reduction occurring in cirrhotic patients. 10 The use of HBV vaccination has resulted in significant declines in the incidence of HCC from HBV. The East Asian neonatal vaccination program is estimated to result in a 70%-85% decrease in the incidence of hepatitis B-related HCC. Despite perinatal immunization, 5%-10% of infants remain at risk of acquiring hepatitis B infection. The use of nucleoside analogs in treating chronic hepatitis B mothers in their third trimester of pregnancy has demonstrated superiority to vaccination alone in preventing neonatal transmission.

Hepatitis C virus (HCV) is a small, single-stranded RNA virus, which exhibits high genetic variability. There are six different genotypes of HCV isolated. Genotypes I, II, and III are predominant in the Western countries and the Far East, while type IV is predominant in the Middle East. The highest rates of chronic hepatitis C infection occur in Egypt (18%), with lower rates occur in Europe (0.5%-2.5%), the United States (1.8%), and Canada (0.8%).16 Once infected with HCV, 80% of patients progress to chronic hepatitis, with ˜20% developing cirrhosis. In Hepatitis C, the development of HCC occurs almost exclusively in the liver with established cirrhosis; however, in the HALT-C trial, 8% of HCC occurred in patients with only advanced fibrosis. Dual infection with HBV and HCV in a cirrhotic patient increases the risk of HCC with an odds ratio (OR) of 165 compared to 17 for hepatitis C and 23 for hepatitis B alone. A synergistic effect with alcohol increases the incidence of HCC between 1.7- and 2.9-fold when compared to HCV-HCC alone. The risk of HCC is reduced significantly in patients who obtained a sustained viral response after treatment of HCV with a 54% reduction in all-cause mortality. While advances in medications recently have made treating HCV easier, vaccinations against the virus remain elusive.

Alcohol consumption remains an important risk factor for the development of HCC. The relationship between alcohol and liver disease correlates with the amount of alcohol consumed over a lifetime, with heavy alcohol use rather than social drinking being the main risk of HCC. The prevalence rate of alcohol abuse in the United States is five times higher than that of hepatitis C. Alcohol abuse accounts for 40%-50% of all HCC cases in Europe. Studies in Europe reported an increase in the relative risk of developing liver disease above 7-13 drinks per week in women and 14-27 drinks per week in men. In the United States, studies showed that the risk of liver cancer is increased two-to fourfold among persons drinking more than 60 g/d of ethanol. A meta-analysis of 19 prospective studies showed that consumption of three or more drinks per day resulted in a 16% increase risk of liver cancer and consumption of six or more drinks per day resulted in a 22% increase risk.

Sixty percent of patients older than 50 years with diabetes or obesity are thought to have NASH with advanced fibrosis. Chronic medical conditions such as diabetes mellitus and obesity increase the risk of HCC. Diabetes mellitus directly affects the liver because of the essential role the liver plays in glucose metabolism. It can lead to chronic hepatitis, fatty liver, liver failure, and cirrhosis. Diabetes is an independent risk factor for HCC. Patients with diabetes have between a 1.8- and 4-fold increased risks of HCC. It is well-known that obesity is associated with many hepatobiliary diseases, including nonalcoholic fatty liver disease (NAFLD), steatosis, and cryptogenic cirrhosis all of which can lead to the development of HCC.

### 2. Other Cancers

In some embodiments, methods of administering a kinase inhibitor in combination with an NDMAR inhibitor are effective for treatment of multiple types of cancer.

It has been established that an NMDAR inhibitor, or a derivative, analog or prodrug, or a pharmacologically active salt thereof in combination with one or more inhibitors of receptor tyrosine kinase can give rise to profound greater than additive killing of cancers that are associated with increased activities and/or gene expression of one or more components in the signaling pathways involved in cancer stemness (including Nestin and Tert), specifically genes involved in the Wnt/β-Catenin signaling pathway including Lgr5 and Axin2. Therefore, multiple non-hormonal cancers can be treated using the compositions and methods described herein.

The combination is particularly effective in treating cancers characterized by up-regulated expression of genes that are involved in cancer sternness (including Nestin and Tert), specifically genes involved in the Wnt/β-Catenin signaling pathway including Lgr5 and Axin2. Therefore, in some embodiments, methods of administering a kinase inhibitor in combination with an NDMAR inhibitor are effective for treatment of cancers characterized by up-regulated expression of genes involved in the Wnt/0-Catenin signaling pathway. In particular embodiments, the methods are effective for treatment of cancers characterized by up-regulated expression of Lgr5 and Axin2. Cancers that have been identified as having up-regulated expression of genes that are involved in cancer sternness (including Nestin and Tert), specifically genes involved in the Wnt/β-Catenin signaling pathway include multiple types of cancers, including but not limited to renal cell carcinoma, thyroid cancer, and FLT3-ITD positive acute myeloid leukemia.

### IV. Kits

Medical kits are also disclosed. The medical kits can include, for example, a dosage supply of an NMDAR inhibitor, a kinase inhibitor, or a combination thereof in separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

The disclosed compositions and methods can be further understood through the following numbered paragraphs.

1. A pharmaceutical composition comprising an effective amount of the combination of an N-methyl-D-aspartate receptor (NMDAR) inhibitor and a kinase inhibitor, wherein administration of the pharmaceutical composition reduces cancer cell proliferation or reduces cancer cell viability, or reduces both cancer cell viability and proliferation in a subject with cancer to a greater degree than administering to the subject the same amount of the NMDAR inhibitor alone or the same amount of the kinase inhibitor alone.
2. The pharmaceutical composition of paragraph 1, wherein the reduction in cancer cell proliferation and/or viability in the subject with cancer is more than the additive reduction achieved by administering the NMDAR inhibitor alone or the kinase inhibitor alone.
3. The pharmaceutical composition of paragraph 1 or 2, wherein the NMDAR inhibitor is selected from the group consisting of ifenprodil, 4-Chlorokynurenine, 7-Chlorokynurenic acid, Kynurenic acid, Phenylalanine, AP5, AP7, CGP-37849, Kaitocephalin, LY-235959, Midafotel, PEAQX, Perzinfotel, Selfotel; dizocilpine, Delucemine, Dextromethorphan, Dextrorphan, Dexanabinol, Diphenidine, Dizocilpine, Esketamine, Ketamine, Lanicemine, Memantine, Methoxetamine, Phencyclidine, Tiletamine, and Amantadine, Diethyl ether, Eliprodil, Hodgkinsine, Nitrous oxide, Psychotridine, Traxoprodil, Xenon, Atomoxetine, Dextropropoxyphene, Ethanol, Guaifenesin, Huperzine A, Ibogaine, Ketobemidone, Methadone, Minocycline, and Tramadol.
4. The pharmaceutical composition of any one of paragraphs 1-3, wherein the NMDAR inhibitor is ifenprodil, a pharmaceutically acceptable salt of ifenprodil, a prodrug, analog, or derivative of ifenprodil, or a pharmaceutically acceptable salt of a prodrug, analog, or derivative of ifenprodil.
5. The pharmaceutical composition of paragraph 4, wherein the dosage of ifenprodil is 1 mg-100 mg.
6. The pharmaceutical composition of any one of paragraphs 1-5, wherein the kinase inhibitor is a receptor tyrosine kinase inhibitor.
7. The pharmaceutical composition of any one of paragraphs 1-6, wherein receptor tyrosine kinase inhibitor is an inhibitor of Fibroblast Growth Factor Receptor or Fms-related tyrosine kinase 4.
8. The pharmaceutical composition of any one of paragraphs 1-7, wherein the kinase inhibitor is selected from the group consisting of sorafenib, lenvatinib, infigratinib, erdafitinib, SAR131675, crizotinib, ceritinib, alectinib, brigatinib, bosutinib, dasatinib, imatinib, nilotinib, vemurafenib, dabrafenib, ibrutinib, palbociclib, ribociclib, cabozantinib, gefitinib, erlotinib, lapatinib, vandetanib, afatinib, osimertinib, ruxolitinib, tofacitinib, trametinib, axitinib, toceranib, nintedanib, pazopanib, regorafenib, sunitinib, dacomitinib, and ponatinib.
9. The pharmaceutical composition of any one of paragraphs 1-8, wherein the kinase inhibitor is sorafenib.
10. The pharmaceutical composition of paragraph 9, wherein the dosage of sorafenib is 100 mg to 1000 mg.
11. The pharmaceutical composition of any one of paragraphs 1-10, wherein the cancer cells are hepatocellular carcinoma.
12. The pharmaceutical composition of any one of paragraphs 1-11, wherein the cancer cells have aberrant Wnt/β-catenin signaling and/or Cdk2 signaling compared to non-cancerous cells.
13. A method of treating cancer comprising administering to a subject with cancer an effective amount of an N-methyl-D-aspartate receptor (NMDAR) inhibitor in combination with an effective amount of a kinase inhibitor, wherein administration of the combination the NMDAR inhibitor and the kinase inhibitor reduces cancer cell proliferation and/or viability in the subject with cancer to a greater degree than administering to the subject the same amount of NMDAR inhibitor alone or the same amount of the kinase inhibitor alone.
14. The method of paragraph 13, wherein the reduction in cancer cell proliferation and/or viability in the subject with cancer is more than the additive reduction achieved by administering the NMDAR inhibitor alone or the kinase inhibitor alone.
15. The method of paragraph 13 or 14, wherein the NMDAR inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the kinase inhibitor to the subject.
16. The method of paragraph 13 or 14, wherein the kinase inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the NMDAR inhibitor to the subject.
17. The method of any one of paragraphs 13-16, further comprising surgery or radiation therapy.
18. The method of any one of paragraphs 13-17, wherein the cancer to be treated is characterized by expression of genes involved in cancer sternness, Wnt/β-catenin signaling pathway, and/or Cdk2 signaling pathway.
19. The method of any one of paragraphs 13-18, wherein one or more genes involved in cancer stemness, Wnt/β-catenin signaling pathway, and/or Cdk2 signaling pathway are selected from the group consisting of Lgr5, Axin2, HES1, AFP, NES, and Tert.
20. The method of any one of paragraphs 13-19, wherein the cancer is characterized by down regulation of expression of one or more genes selected from the group consisting of Lgr5, Axin2, HES1, AFP, NES, and Tert following treatment.
21. The method of any one of paragraphs 13-20, wherein the cancer is characterized by up-regulation of unfolded protein response effectors including phosphorylated IRE1-alpha and/or C/EBP Homologous Protein (CHOP) following treatment.
22. The method of any one of paragraphs 13-21, further comprising the step of selecting a subject having a cancer characterized by overexpression of one or more genes involved in cancer stemness, Wnt/β-catenin signaling pathway, and/or Cdk2 signaling pathway.
23. The method of any one of paragraphs 13-22, wherein the NMDAR inhibitor and the kinase inhibitor are administered in an amount effective to reduce the serum concentration of one or more of Lens culinaris-reactive AFP, Golgi Protein 73, Asialo-alpha-acid glycoprotein, laminin, neopterin, and Glypican-3 compared to the serum concentration of one or more of Lens culinaris-reactive AFP, Golgi Protein 73, Asialo-alpha-acid glycoprotein, laminin, neopterin, and Glypican-3 prior to treatment.

The present invention is further understood by reference to the following non-limiting examples.

### EXAMPLES

### Example 1: A CRISPR-Cas9 Screen Identifies Combinatorial Therapeutic Targets that Inhibit HCC Growth

### Materials and Methods

Cell Culture

Human HCC cell lines (HepG2 and Hep3B) and HEK293T were purchased from American Type Culture Collection (ATCC). The sorafenib-resistant HepG2 cells were prepared according to inventors' previous work (22). Human HCC cell line Huh7 was purchased from the JCRB Cell Bank. HepaRG cells were purchased from ThermoFisher Scientific. Human HCC cell line MHCC97L was obtained from Liver Cancer Institute, Fudan University. Human liver cell line L02 was obtained from the Institute of Virology, Chinese Academy of Medical Sciences, Beijing, China. RIL-175 cell line was obtained from the National Cancer Institute (National Institutes of Health, Bethesda, USA). MHCC97L, HepG2, Huh7, Hep3B, L02, and HEK293T were maintained in Dulbecco's Modified Eagle Medium (DMEM). HepaRG cells were maintained in William's E Medium supplemented with GlutaMAX Supplement (100X) and HepaRG Maintenance/Metabolism Medium Supplement (5X). To generate cell lines stably expressing Cas9 protein (i.e., MHCC97L-Cas9), MHCC97L cells were infected with lentiviral pAWp30 (Addgene, 73857) vector, followed by 2 weeks of selection with Zeocin (200 μg/ml, ThermoFisher Scientific). All cells were grown in complete medium supplemented with 10% fetal bovine serum (FBS) and 1% antibiotic-antimycotic (ThermoFisher Scientific) at 37° C. with 5% CO₂. To generate the UPR-depleted cells, MHCC97L-Cas9 cells were infected with vectors expressing sgRNAs targeting IRE1-alpha, PERK, and ATF6, and cultured for 6 days prior to downstream assays. Cells were regularly tested for mycoplasma contamination using PCR detection and were confirmed to be negative.

Organoid Culture

HCC patient-derived organoids labelled HCC #23 and HCC #10 were gifts from Meritxell Huch of The Gurdon Institute at the University of Cambridge. HCC organoid lines, named as HCC-HK P1 (or KYM) and HCC-HK P2 (or LKY), were established from resected HCC tissues of HCC patients. Four HCC organoid lines, named as HCC-HK P1, HCC #23, HCC #10, and HCC-HK P2, were prepared from HCC patients undergoing hepatectomy at Queen Mary Hospital, Hong Kong SAR. Samples were collected from patients who had not received any previous local or systemic treatment prior to operation. Informed consent was obtained from all patients before the collection of liver specimens and the study was approved by the Ethics Committee of the University of Hong Kong. HCC cells were mixed with Matrigel (BD Biosciences), and 10,000 cells were seeded per well onto a 24-well plate for culture. After Matrigel had solidified, culture medium was added. The culture medium was based on AdDMEM/F12 (ThermoFisher Scientific), supplemented with 1% N2 and 1% B27 (both from ThermoFisher Scientific), 1.25 mM N-Acetyl-L-cysteine (Sigma-Aldrich), 10 nM gastrin (Sigma-Aldrich), and the growth factors including 50 ng/ml EGF (Sigma-Aldrich), 10% RSPO1 conditioned media (homemade), 100 ng/ml FGF10 (Sigma-Aldrich), 25 ng/ml HGF (Peprotech), 10 nM Nicotinamide (Sigma-Aldrich), 5 μM A83.01 (Tocris), and 10 μM Forskolin (FSK) (Tocris). The organoids were maintained at 37° C. with 5% CO₂.

Mouse

Four to five week-old male BALB/c nude mice and NOD/SCID mice were used in the experiments. All experimental procedures were approved by the Committee of the Use of Live Animals in Teaching and Research at The University of Hong Kong and the Animals (Control of Experiments) Ordinance of Hong Kong, and performed in compliance with the institutional guidelines.

Characterization of HCC Organoids

Cells isolated from both non-tumor liver and HCC tissue were sustainably expanded in vitro for more than 6 months, with budding- and cystic-like morphology; and were passaged continuously or frozen/thawed for long-term storage. Histology of the organoids closely mimic that of the unprocessed clinical tissue specimen, with HCC phenotype validated by presence of both alpha fetoprotein (AFP) and glypican 3 (GPC3) as well as the absence of cytokeratin 19 (CK19) by immunohistochemistry. Nuclear pleomorphism in both HCC tissue and corresponding organoid sections were also clearly noted. Early (passage 2) and late passages (passage 12, >5 months in culture) of non-tumor liver and HCC patient-derived 3D organoids against the corresponding tissue biopsies were thoroughly analyzed and compared by whole exome sequencing (WES), to ensure that the organoid cultures recapitulated their corresponding biopsies at the genetic level. Specifically, only 3 base substitutions were observed in protein-coding DNA of non-tumor liver organoids, of which the frequency was comparable to previous studies. On the other hand, using the same filtering criteria, only 17 protein-coding base substitutions were found in HCC tumor organoid cultures, of which 3 were introduced during long-term passage. Above named mutations were not identified in any known HCC drive genes or COSMIC70 databases. In sum, both the immunohistochemistry and the WES data lend support to the fact that the organoid cultures are genetically stable and highly recapitulate the molecular features of the original tumor.

Plasmid Construction

The plasmids used in this study were purchased from Addgene or generated by standard molecular cloning strategies, including oligo annealing, PCR, and restriction enzyme digestion (Table 1). Custom oligonucleotides were purchased from Genewiz. DH5a competent E. coli cells were used for transformation and positive colonies were selected with ampicillin (100 μg/ml, USB) or carbenicillin (50 μg/ml, Teknova). DNA was extracted and purified by Plasmid Mini (Takara and Tiangen) or Midi preparation (Qiagen) kits. Sequences of the vectors were confirmed by Sanger sequencing.

TABLE 1
List of plasmid constructs

| Construct ID | Detail |
|---|---|
| pAWp92 | pBT264-mutH1p-scaffold |
| pAWp28 | pBT264-U6p-{2xBbsI}-sgRNA scaffold-{MfeI} |
| pAWp103 | pFUGW-EFSp-EGFP |
| pAWp30 | pFUGW-EFSp-SpCas9-P2A-Zeo |
| pWXp63 | pFUGW-EFSp-EGFP-U6p-FLT4sg1-v1 scaffold |
| pWXp64 | pFUGW-EFSp-EGFP-U6p-FLT4sg3-v1 scaffold |
| pWXp66 | pFUGW-EFSp-EGFP-mutH1p-GRIN1sg1-wt scaffold |
| pWXp67 | pFUGW-EFSp-EGFP-mutH1p-GRIN1sg2-wt scaffold |
| pWXp69 | pFUGW-EFSp-EGFP-U6p-FGFR3sg2-v1 scaffold |
| pWXp72 | pFUGW-EFSp-EGFP-U6p-FGFR3sg1-v1 scaffold |
| pWXp73 | pFUGW-EFSp-EGFP-mutH1p-FGFR3sg1-wt scaffold |
| pWXp74 | pFUGW-EFSp-EGFP-mutH1p-FGFR3sg3-wt scaffold |
| pWXp78 | pFUGW-EFSp-EGFP-mutH1p-GRIN1sg2-wt scaffold-U6p-FLT4sg1-v1- |
| | scaffold |
| pWXp79 | pFUGW-EFSp-EGFP-mutH1p-GRIN1sg1-wt scaffold-U6p-FLT4sg3-v1- |
| | scaffold |
| pWXp85 | pFUGW-EFSp-EGFP-mutH1p-GRIN1sg2-wt scaffold-U6p-FGFR3sg1-v1- |
| | scaffold |
| pWXp86 | pFUGW-EFSp-EGFP-mutH1p-GRIN1sg2-wt scaffold-U6p-FGFR3sg2-v1- |
| | scaffold |
| pWX120 | pFUGW-CMVp-EGFP-U6-PERKsg1-wt scaffold |
| pWX122 | pFUGW-CMVp-EGFP-U6-IRE1sg1-wt scaffold |
| pWX124 | pFUGW-CMVp-EGFP-U6-ATF6sg1-wt scaffold |
| PZp137-1 | pFUGW-CMVp-EGFP-mutH1p-PPP1R12Csg-wt scaffold-U6p-THUMPD3- |
| | AS1sg1-v1 scaffold |
| PZp137-2 | pFUGW-CMVp-EGFP-mutH1p-PPP1R12Csg-wt scaffold-U6p-THUMPD3- |
| | AS1sg2-v1 scaffold |
| Addgene #73857 | pAWp30 |
| Addgene #11976 | p5xATF6-GL3 |
| Addgene #24307 | 7TFC |

Single Guide RNA Design and Library Assembly

Ninety sgRNAs that target 30 selected genes (3 sgRNAs per gene, Table 2) were selected, and three sgRNAs that do not target any known DNA sequence in the human genome were used as controls. The sgRNA sequences were designed based on the sgRNA Designer (BROAD Institute, Massachusetts Institute of Technology). Several criteria were applied to select sgRNAs with: 1) on-target score (Azimuth2.0)>0.6; 2) off-target ranks<180; 3) target sites within 5-65% of the protein-coding sequence; 4) no BbsI, BgIII, Mfe, BaNHI, and EcoRI digestion sites. The barcoded pairwise sgRNA library was assembled using CombiGEM-CRISPR v2.0 as described previously (Wong et al. (2016), Annual Rev Genet 50: pages 515-538; Zhou et al. (2020) Cell Reports 32(6): 108020). In brief, pAWp103 lentiviral vector was digested by BamHI and EcoRI, and the pooled barcoded sgRNA inserts were released from storage vector libraries through BgIII and MfeI digestion. Via base pairing of the compatible sticky ends (BamHI+BgIII and EcoRI+MfeI), the insert pool with mutH1-sgRNA-WT scaffold was ligated into the lentiviral vector to generate the mutH1-sgRNA-WT scaffold library. To generate the pairwise sgRNA library (93×93 sgRNAs=8,649 total combinations), the barcoded sgRNA insert pool released from the U6-sgRNA-v1 scaffold storage library was ligated to the mutH1-sgRNA-WT scaffold library digested with BamHI and EcoRI. The barcodes representing the two sgRNA inserts were concentrated on one end in the assembled library construct and were read via Illumina sequencing.

TABLE 2
List of genes in this study

| | | On-target | Off- | SEQ |
|---|---|---|---|---|
| | | (Azimuth2.0) | target | ID |
| gRNA | gRNA target sequence | score | rank | NO: |
| dummysg1 | ATCGTTTCCGCTTAACGGCG | / | / | 1 |
| dummysg2 | AAACGGTACGACAGCGTGTG | / | / | 2 |
| dummysg3 | CCATCACCGATCGTGAGCCT | / | / | 3 |
| ADORA2Asg1 | CTATTTGCGGATCTTCCTGG | 0.7285 | 102 | 4 |
| ADORA2Asg2 | AAGCAGTTGATGATGTGTAG | 0.6711 | 60 | 5 |
| ADORA2Asg3 | TGGCTTGGTGACCGGCACGA | 0.6571 | 35 | 6 |
| ANXA1sg1 | AGAAATCAGAGACATTAACA | 0.7146 | 11 | 7 |
| ANXA1sg2 | AAAATCTCCAGATGTGTCTG | 0.6654 | 63 | 8 |
| ANXA1sg3 | TCACACCAAAGTCCTCAGAT | 0.6498 | 9 | 9 |
| ANXA3sg1 | CTGAGAGGTCAAATGCACAG | 0.8196 | 13 | 10 |
| ANXA3sg2 | TTTGCATCAAAGACTGCTGG | 0.6396 | 10 | 11 |
| ANXA3sg3 | GCTATTCAGAAAGCAATCAG | 0.6807 | 65 | 12 |
| CFTRsg1 | AACGTGTTGAGGGTTGACAT | 0.6877 | 8 | 13 |
| CFTRsg2 | TGTGGACAGTAATATATCGA | 0.6545 | 4 | 14 |
| CFTRsg3 | CCACGCTTCAGGCACGAAGG | 0.654 | 3 | 15 |
| DPP4sg1 | GGATTCCAAACAACACACAG | 0.735 | 25 | 16 |
| DPP4sg2 | CTACTTGTGTGATGTGACAT | 0.6877 | 19 | 17 |
| DPP4sg3 | ATTTATTACAAAAGGCACCT | 0.6537 | 89 | 18 |
| FGAsg1 | GGTTGATATGAAACGACTGG | 0.7661 | 44 | 19 |
| FGAsg2 | ACAGTCAGAACCATCTTCGG | 0.6878 | 10 | 20 |
| FGAsg3 | TGATCCGGTTCCATAAGAGG | 0.6753 | 18 | 21 |
| FGFR2sg1 | CTTAGTCCAACTGATCACGG | 0.7903 | 13 | 22 |
| FGFR2sg2 | TGACCAAACGTATCCCCCTG | 0.7632 | 57 | 23 |
| FGFR2sg3 | GCCGGCAAATGCCTCCACAG | 0.7462 | 91 | 24 |
| FGFR3sg1 | AAGAACGGCAGGGAGTTCCG | 0.7311 | 33 | 25 |
| FGFR3sg2 | GGTGCTGAATGCCTCCCACG | 0.6882 | 32 | 26 |
| FGFR3sg3 | CATCCGGCAGACGTACACGC | 0.6538 | 2 | 27 |
| FLT4sg1 | CTCACCTCTCACGAACACGT | 0.7463 | 57 | 28 |
| FLT4sg2 | GCCCTCCAGTCACGGCACTG | 0.704 | 42 | 29 |
| FLT4sg3 | CATACCATGCACAATGACCT | 0.6988 | 92 | 30 |
| NMDAR1sg1 | AGCCGTTCCAGAGCACACTG | 0.787 | 108 | 31 |
| NMDAR1sg2 | CAAAAGCCGTAGCAACACTG | 0.7447 | 31 | 32 |
| NMDAR1sg3 | CGGGCAGGCAGACATGATCG | 0.6612 | 71 | 33 |
| NMDAR3Bsg1 | CCATGACATTGTGCAACTGG | 0.6679 | 45 | 34 |
| NMDAR3Bsg2 | GGGGCTACGCCACTCGTACA | 0.6546 | 19 | 35 |
| NMDAR3Bsg3 | AGGTACAGCTCGAAGTCGAA | 0.6519 | 21 | 36 |
| HDAC9sg1 | AGCTTTGATCCAATGATGTG | 0.6955 | 12 | 37 |
| HDAC9sg2 | AACAGCATGAGAACTTGACA | 0.6739 | 18 | 38 |
| HDAC9sg3 | TTTCCCTCTAAAGTAACATG | 0.6593 | 17 | 39 |
| ICAM1sg1 | CGAAGCCAGAGGTCTCAGAA | 0.7316 | 136 | 40 |
| ICAM1sg2 | TGACGTGTGCAGTAATACTG | 0.6888 | 14 | 41 |
| ICAM1sg3 | GCCCGCTGAGGTCACGACCA | 0.6418 | 20 | 42 |
| IL8(CXCL8)sg1 | ATTTCTGTGTTGGCGCAGTG | 0.6982 | 1 | 43 |
| IL8(CXCL8)sg2 | CAGAGCTGCAGAAATCAGGA | 0.6608 | 14 | 44 |
| IL8(CXCL8)sg3 | ATTTCTGCAGCTCTGTGTGA | 0.6155 | 23 | 45 |
| ITGB7sg1 | AGAGTGTTCAAGGGTCACGG | 0.7577 | 94 | 46 |
| ITGB7sg2 | CCACGTCCGAATCAACCAGA | 0.7127 | 6 | 47 |
| ITGB7sg3 | CCGGGTATCCCTCAGCACGA | 0.6932 | 33 | 48 |
| KCND3sg1 | TCTGGTCATGACCAACAACG | 0.7746 | 38 | 49 |
| KCND3sg2 | GTGCGTCATGATCTTCACCG | 0.816 | 172 | 50 |
| KCND3sg3 | GACAATGGTGTACCAAAACG | 0.6906 | 14 | 51 |
| KITsg1 | GCCTAATCTCGTCGCCCACG | 0.7234 | 5 | 52 |
| KITsg2 | ATTACGAAACCAATCAGCAA | 0.6676 | 34 | 53 |
| KITsg3 | GAATGGCATGCTCCAATGTG | 0.6578 | 27 | 54 |
| MAPK11sg1 | CCCCTACCAGACGGAGCCGT | 0.6088 | 66 | 55 |
| MAPK11sg2 | GGTGGATGATCCCGGCCGAG | 0.646 | 47 | 56 |
| MAPK11sg3 | TGACCAGCTGAAGCGCATCA | 0.6162 | 70 | 57 |
| NPR1sg1 | GATCGACGTTAGCTCCAACG | 0.7838 | 1 | 58 |
| NPR1sg2 | GTGTATTAGGTATGACTACG | 0.7343 | 69 | 59 |
| NPR1sg3 | GCTGTCGGTGTAAAAAACCA | 0.7119 | 25 | 60 |
| ODC1sg1 | ATCAGAGATTGCCTGCACGA | 0.6971 | 34 | 0 |
| ODC1sg2 | CAACGCTGGGTTGATTACGC | 0.6635 | 2 | 62 |
| ODC1sg3 | GAAGGGGCTTTACATGTGCG | 0.6543 | 31 | 63 |
| OXTRsg1 | GCGGCTCAAGACCGCTGCAG | 0.7056 | 40 | 64 |
| OXTRsg2 | CCTGCAAGTACTTGACCAGG | 0.6967 | 32 | 65 |
| OXTRsg3 | GCTAGCTGTCTACATCGTGC | 0.6589 | 25 | 66 |
| PDGFRAsg1 | CGAGACAGGAGTACCGTGGA | 0.6822 | 43 | 67 |
| PDGFRAsg2 | TAAGTCAGGGGAAACGATTG | 0.6818 | 4 | 68 |
| PDGFRAsg3 | CTGGTCATTTATAGAAACCG | 0.6815 | 21 | 69 |
| PROCsg1 | AATTGCTCGCTGGACAACGG | 0.7171 | 3 | 70 |
| PROCsg2 | ACGTGCCAACTCCTTCCTGG | 0.7007 | 68 | 71 |
| PROCsg3 | GCCTTCTGGTCCAAGCACGT | 0.6508 | 2 | 72 |
| PTGER4sg1 | CAGCGCGCAAAAGAGCACGT | 0.7 | 7 | 73 |
| PTGER4sg2 | GCCCGCGTACATGTAGGAGT | 0.6548 | 2 | 74 |
| PTGER4sg3 | GTTCACAGAAGCAATTCGGA | 0.6539 | 41 | 75 |
| PTK7sg1 | CTGCAACATCAAGCACACGG | 0.7614 | 112 | 76 |
| PTK7sg2 | GCTCTGACCATCAGAAAGGG | 0.7015 | 45 | 77 |
| PTK7sg3 | GAGCGTACGACTGTGTACCA | 0.6821 | 5 | 78 |
| SERPINE1sg1 | AGGGTGAGAAAACCACGTTG | 0.8278 | 7 | 79 |
| SERPINE1sg2 | CAGACGCGATCTTCGTCCAG | 0.6998 | 2 | 80 |
| SERPINE1sg3 | GCTGAGTTCACCACGCCCGA | 0.6566 | 29 | 81 |
| TNFsg1 | TTGGAGTGATCGGCCCCCAG | 0.6809 | 11 | 82 |
| TNFsg2 | AGAGCTCTTACCTACAACAT | 0.6335 | 13 | 83 |
| TNFsg3 | GGAGCTGAGAGATAACCAGC | 0.6231 | 18 | 84 |
| TOP2Asg1 | TGTACGCTTATCCTGACTGA | 0.6806 | 50 | 85 |
| TOP2Asg2 | AGCATTGTAAAGATGTATCG | 0.6731 | 21 | 86 |
| TOP2Asg3 | TGAACAAGTAAACCACAGGT | 0.6623 | 142 | 87 |
| TRPV3sg1 | TGTACGACATGATCCTACTG | 0.7378 | 4 | 88 |
| TRPV3sg2 | AACCCCAAGTACCAACACGA | 0.7226 | 15 | 89 |
| TRPV3sg3 | AGGATGGACTGCAGATCCGA | 0.6768 | 18 | 90 |
| TUBA1Asg1 | CTGTGATAAGTTGCTCAGGG | 0.7492 | 23 | 91 |
| TUBA1Asg2 | TGCGAATTCGGTCCAACACG | 0.6743 | 47 | 92 |
| TUBA1Asg3 | AAGTCTACAAACACTGCCCG | 0.6647 | 55 | 93 |
| ATF6sg1 | TTGTAGGACAGGTTTAGTCA | 0.6647 | 33 | 100 |
| IRE1sg1 | CATGTTTGACAACCGCGACG | 0.7014 | 1 | 101 |
| PERKsg1 | TTATCTACCATACTACAAGA | 0.7108 | 36 | 102 |
| PPP1R12Csg | GGGTAAACCGACTCCCCCGA | 0.6583 | 32 | 103 |
| THUMPD3- | CAGGTAAAACTGACGCACGG | 0.7354 | 42 | 104 |
| AS1sg1 | | | | |
| THUMPD3- | GGGACCACCTTATATTCCCA | 0.7134 | 30 | 105 |
| AS1sg2 | | | | |

Lentiviral Vector Generation and Transduction

The second-generation lentiviral vector system was used in this study. For each well of a 6-well plate, 1 μg of pCMV-dR8.2-dvpr vector, 0.5 μg of pCMV-VSV-G vector, and 0.5 μg of the FUGW-based lentiviral vector was mixed in pre-warmed Opti-MEM medium (ThermoFisher Scientific). FuGENE® HD (Promega) was used for transfecting the vector mix into HEK293T cells following the manufacturer's protocol. The culture medium was replaced with new culture medium on the next day. The viral medium was collected at 48 hours and 72 hours after transfection. The two batches of viral media were combined and filtered through a 0.45 μm polyethersulfone membrane (Pall, NY, USA). For individual vector transduction, 200 μl of the filtered virus medium was added into each well of a 12-well plate at ˜30% cell confluency in the presence of 8 μg/ml polybrene (Sigma-Aldrich). For pooled library transduction, a multiplicity of infection (MOI) of ˜0.3 was used and no polybrene was added. To ensure high representation of each library member in the infected cell pool, the starting number of cells used for the transduction was ˜500-fold of the library size.

Flow Cytometry, Cell Cycle Analysis, and Cell Sorting

Cells transduced with florescent proteins were trypsinized, washed with PBS, and resuspended in PBS containing 2% FBS. Resuspended cells were subjected to flow cytometry analysis using the BD FACSCantoII, ACEA NovoCyte Quanteon (Agilent) or BD LSR Fortessa Analyzer (BD Biosciences). The signal of TurboRFP and EGFP was detected by a 561 nm yellow-green laser (610/20 nm) and a 488 nm blue laser (530/30 nm), respectively. For cell cycle analysis, cells harvested by trypsinization were washed by PBS. Cell pellets were fixed by ice-cold 70% ethanol at 4° C. for 1 hour. The fixed cells were rehydrated by replacing ethanol with PBS for 15 minutes at room temperature for cell cycle analysis. To remove RNAs, 5 μl RNase A (10 mg/ml) per 500 μl PBS was added and incubated at 37° C. for 15 minutes. The DNA contents were stained by propidium iodide (ThermoFisher Scientific) for 1 hour at room temperature in dark. Signal was detected by a 561 nm yellow-green laser (581/15 nm). FlowJo software (v10.5.3, Becton Dickinson) was used for data analysis. For cell sorting, cells were collected following the normal trypsinization and PBS washing steps. Cell pellets were resuspended in PBS with 2% FBS and 2X antibiotic-antimycotic. Resuspended cells were subjected to cell sorting using the BD Influx cell sorter (BD Biosciences) equipped with 100-μm nozzle. Signal was detected by a 488 nm blue laser (530/40 nm). ˜500-fold more cells were sorted into culture medium supplemented with 10% FBS and 2X antibiotic-antimycotic. The culture medium was changed with fresh complete culture medium (10% FBS and 1X antibiotic-antimycotic) 1 day after sorting.

Annexin V Staining

For Annexin V staining, cells were seeded onto a 10 -cm dish and treated with 10 μM ifenprodil, 5 μM sorafenib, or both. After 24 hours of drug treatment, cells were stained with Annexin V kit (Biolegend, #640920) following the manufacturer's protocol. Briefly, cells were washed with PBS twice and resuspended with 1X Binding Buffer. 5 μl per ˜1×10⁵ cells of APC Annexin V was added and incubated for 15 minutes at room temperature in the dark. Cells were then resuspended in 1 X Binding Buffer and analyzed using the BD FACS CantoII, ACEA NovoCyte Quanteon (Agilent), or BD LSR Fortessa Analyzer (BD Biosciences). The signal of APC was detected by 640 nm Red Laser (670/14 nm).

Sample Preparation for Next-Generation Sequencing

Genomic DNA from cell pools infected with the sgRNA library was extracted using DNeasy Blood & Tissue Kit (QIAGEN) following the manufacturer's protocol. The DNA concentration was quantified by Quant-iT™ PicoGreen™ dsDNA Assay Kit (ThermoFisher Scientific). To amplify the 298-bp fragment harboring the sgRNA combination-specific barcode and assign an indexing barcode to each sample, two rounds of PCR were carried out. The primers used in the first-round PCR were: 5′-GGATCCGCAACGGAATTC-3′ (forward; SEQ ID NO:94) and 5′-GGTTGCGTCAGCAAACACAG-3′ (reverse; SEQ ID NO:95), which amplify the barcode-containing regions out from the genomic DNA; the primers used in the second-round PCR were: 5′-CAAGCAGAAGACGGCATACGAGATGTGACTGGAGTTCAGACGTGTGCTCTTCC GATCTGGTTGCGTCAGCAAACACAG-3′ (forward; SEQ ID NO:96) and 5′-AATGATACGGCGACCACCGAGATCTACACTCTTTCCCTACACGACGCTCTTCC GATCTNNNNNNNNGGATCCGCAACGGAATTC-3′ (reverse; SEQ ID NO:97), which add Illumina anchor sequences and an indexing barcode (denoted by NNNNNNNN) assigned for each sample. The same set of primers were used for amplifying the barcodes from the plasmid library. 1 ng of plasmid DNA or 800 ng of genomic DNA from cell pools was used as the template for each 50-μl PCR reaction using KAPA HiFi ready mix (Roche). The PCR conditions were optimized for each individual sample to minimize PCR bias. 12 PCR reactions per sample was performed to reach at least 100-fold coverage. The quantity and quality of the PCR products were measured using Real-time PCR with SYBR® Premix Ex Taq™ (TaKaRa). Signal was detected using the StepOnePlus Real Time PCR system (Applied Biosystem). The forward and reverse primers used were 5′-AATGATACGGCGACCACCGA-3′ (SEQ ID NO:98) and 5′-CAAGCAGAAGACGGCATACGA-3′ (SEQ ID NO:99), respectively. Samples were then mixed at a desired ratio depending on the concentration and correspondence library size for multiplexing and were loaded onto the Illumina HiSeq system.

Barcode Sequencing Data Analysis

The barcode reads for individual samples were normalized to count per million reads. The library coverage and reproducibility between biological replicates were analyzed. To increase the confidence in screen hit identification, only combinations that had >=100 raw barcode reads in the baseline time point (Day 0, which is 11 days after transduction, sorting for infected cells, and post-sort recovery) sample and coefficients of variation (CV) of <1 were included in the analysis. Reads for each sgRNA combination were normalized with the averaged read for the control sgRNAs. Log₂ fold change was calculated by comparing the late (day 7) and baseline time point samples. The P-value for each combination was determined for the significance level of the fold change. Gene combinations with at least two sets of sgRNA combinations having mean log₂ ratios of <-0.51 and P-values of <0.05 based on results obtained from two biological replicates are listed in Table 3.

TABLE 3
List of gRNA combinations with mean log2 ratios
of <−0.51 and P-values of <0.05

| | | log- | |
|---|---|---|---|
| | | expression | |
| gRNA-1 | gRNA-2 | (Averaged) | P-Value |
| FGFR3sg1 | ANXA3sg2 | −1.2918 | 0.0326 |
| FGFR3sg3 | ANXA3sg3 | −0.7028 | 0.0488 |
| FLT4sg3 | FGFR2sg3 | −0.6544 | 0.0306 |
| FLT4sg3 | FGFR2sg2 | −2.5940 | 0.0127 |
| NMDAR1sg2 | FGFR3sg1 | −0.9028 | 0.0306 |
| NMDAR1sg2 | FGFR3sg2 | −0.9091 | 0.0069 |
| NMDAR1sg1 | FLT4sg3 | −1.8030 | 0.0498 |
| NMDAR1sg2 | FLT4sg1 | −4.4690 | 0.0098 |
| ITGB7sg1 | ANXA3sg1 | −1.0305 | 0.0288 |
| ITGB7sg1 | ANXA3sg3 | −3.7177 | 0.0153 |
| ITGB7sg3 | CFTRsg3 | −1.0011 | 0.0450 |
| ITGB7sg3 | CFTRsg1 | −1.0620 | 0.0488 |
| PDGFRAsg1 | CFTRsg2 | −1.3845 | 0.0175 |
| PDGFRAsg3 | CFTRsg2 | −1.8778 | 0.0078 |
| PDGFRAsg1 | FLT4sg1 | −1.0818 | 0.0224 |
| PDGFRAsg2 | FLT4sg3 | −0.5169 | 0.0260 |
| PTK7sg1 | NPR1sg2 | −1.9360 | 0.0403 |
| PTK7sg3 | NPR1sg1 | −1.1460 | 0.0418 |
| PTK7sg1 | NMDAR3Bsg3 | −0.6259 | 0.0317 |
| PTK7sg3 | NMDAR3Bsg3 | −0.9441 | 0.0040 |
| SERPINE1sg3 | KCND3sg2 | −1.0645 | 0.0262 |
| SERPINE1sg3 | KCND3sg3 | −0.5290 | 0.0350 |

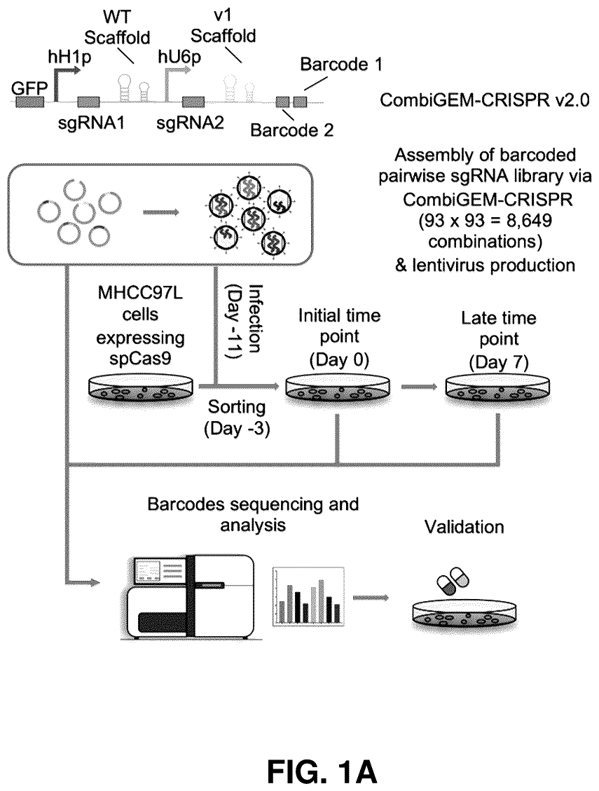

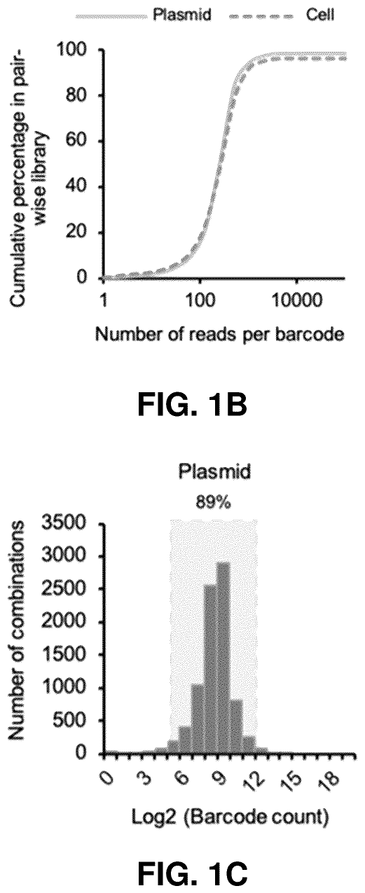

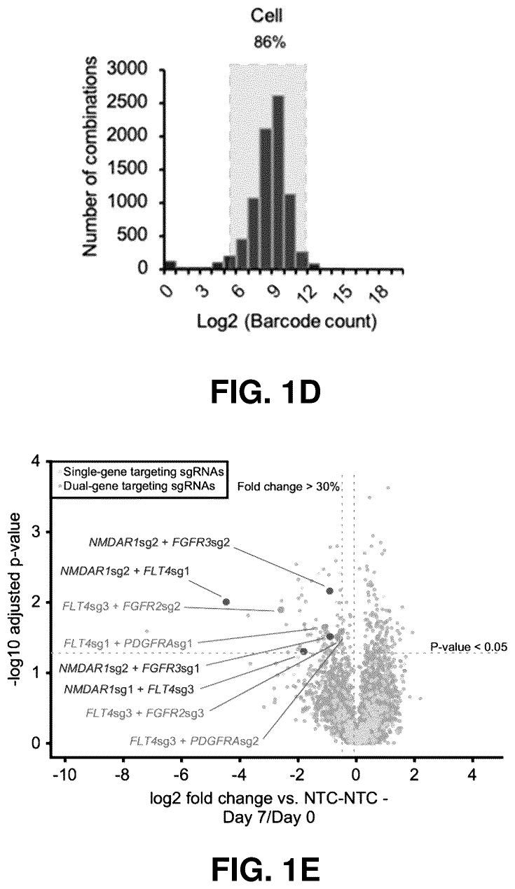

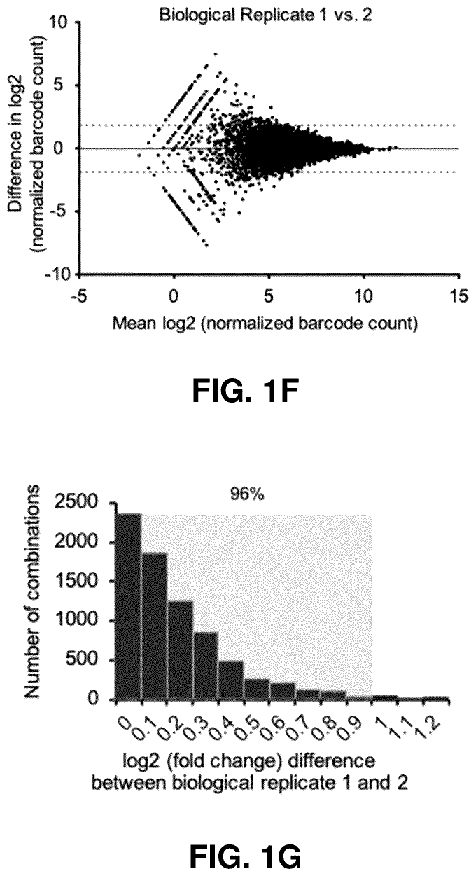

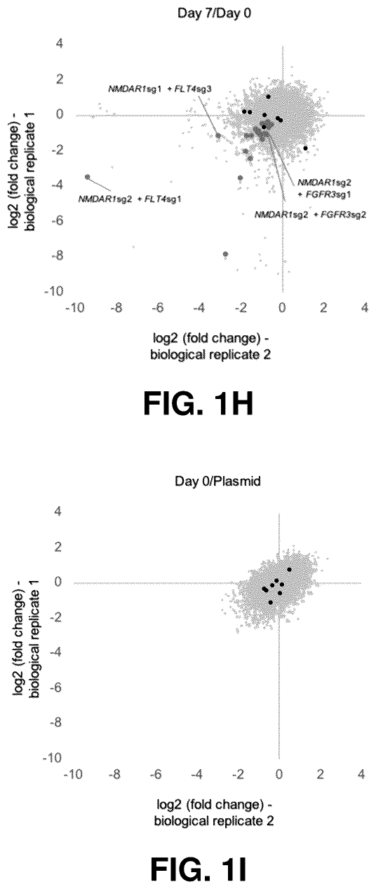

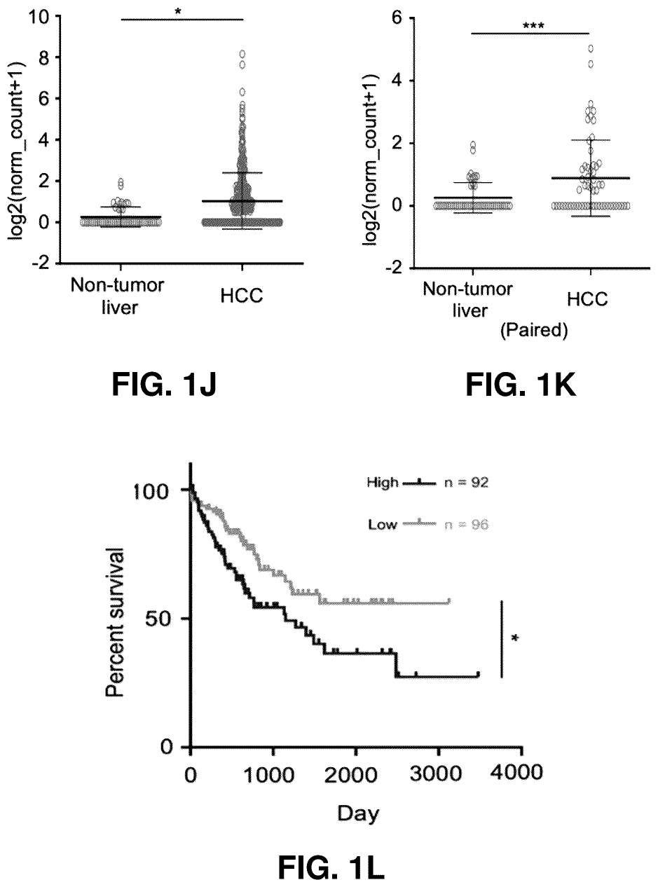

Only relatively minor changes in the representation of combinations were observed between plasmid and day 0 samples (FIGS. 1B-1I), because phenotypes resulting from the targeted gene inactivation take time to manifest.

Cell Viability Assay and Drug Interaction Analysis

To validate the growth inhibition effects brought by the selected sgRNA combinations, MTT assay was applied. On day 7 post-infection, 2,000 lentivirus-transduced HCC cells expressing the sgRNA combinations were seeded onto each well of a 96-well plate. Cell viability was then determined every 24 hours by performing MTT assay according to the manufacturer's protocol. Absorbance was detected at 570 nm and 650 nm (as a reference) by spectrophotometry using the Synergy H1 Microplate Reader (BioTek). To validate the growth inhibition effects brought by the drug(s) in HCC cells, 2,000 HCC cells were seeded onto each well of a 96-well plate, and drugs were added to the cells on the next day. Cell viability was determined by MTT assay after 2 days of drug treatment. To measure the growth inhibition effects brought by the drug(s) in HCC organoids, CellTiter-Glo Luminescent Cell Viability Assay Kit (Promega) was used following the manufacturer's instructions. 1,000 cells were seeded onto each well of a 384-well plate supplemented with Matrigel and complete organoid growth medium, and cultured for 3 days to form organoids. New culture medium together with the 2X concentration of the drug(s) were then added to each well and cultured for another 72 hours. CellTiter-Glo Reagent were added to each well, and incubated at room temperature for 30 minutes. Luminescence was detected using the VICTOR3 Multilabel Plate Reader (PerkinElmer).

Drug Interaction Analysis

The drugs used were ifenprodil (Selleckchem, #54091), infigratinib (LC laboratories, #I-1500), erdafitinib (Selleckchem, #58401), SAR131675 (Sellectchem, #S2842), sorafenib (LC laboratories, #5-8502), lenvatinib (LC laboratories, #L-5400), MK801 (Selleckchem, #S2857), and thapsigargin (Sigma, #586005).

Drug interaction was evaluated by two models: the Bliss independence model (Bliss (1939) Ann. Appl. Bio. (26): pages 585-615) and the Highest Single Agent (HSA) model (Borisy et al. (2003) PNAS 100(13): pages 7977-7982). Based on the Bliss independence model, the expected growth inhibition (Eexp) brought by drug combination (A+B) is given by:

Eexp=Ea+Eb−Ea×Eb

where Ea is the growth inhibition brought by drug A alone, and Eb is the growth inhibition brought by drug B alone. The difference between the expected (Eexp) and the observed (Eobs) growth inhibition was calculated. When ΔE (Eobs−Eexp)>0, the two drugs are considered to be interacting synergistically. Similarly, based on the HSA model, two drugs are considered to act synergistically when ΔE (Eobs−Eexp)>0. In this model, Eexp equals to the higher growth inhibition being induced by the individual drug within the combination (Eexp=max (Ea, Eb)).

It was observed that that the Bliss and HSA scores did not always match, and thus a statistically rigorous definition of synergy was needed. A Bliss independence response surface model (Zhao et al. (2014) J Bimol Screen 19(5): pages 817-21) was further applied to calculate an excess over Bliss score and 95% confidence interval values of the score, using a two-stage modelling. The first stage of modelling calculates the average of observed responses and corresponding variance at each dose of drug A and drug B. The second stage of modelling uses the first stage-estimated means and variances to simulate 100 random samples of the predicted response using multivariate normal distributions. Conditional on the predicted response, a linear model is used to estimate response surface parameters and then the variance of the excess over Bliss score and the 95% confidence interval values of the score. At a given dose combination, the combination effect of the two drugs is considered as significant synergism if the lower confidence bound of the excess over Bliss score is greater than 0.

Drug Response Study in Mice

5×10⁵ MHCC97L cells, 3.5×10⁵ cells (for PDX1) and 1×10⁵ cells (for PDX2) dissociated from HCC patient derived xenograft (PDX), were injected subcutaneously into the left flank of 4 to 5-week-old male BALB/c nude mice and NOD/SCID mice, respectively. 1×10⁴ RIL-175 cells were injected into the right median lobe of the livers from 9 to 12-week-old male C57BL/6N mice. Size of subcutaneously xenografted tumors was measured by an external caliper and tumor volume was estimated by the equation: Volume=½ (Length×Width²), where the length and width represent the largest and smallest diameters, respectively. Drug administration began when the subcutaneous tumor reached ˜0.05 cm3 in size or at day 5 post-injection (for RIL-175 cells), at which point mice were divided into four groups randomly for daily treatment with DMSO, ifenprodil (20 mg/kg/day, intraperitoneally), sorafenib (28 mg/kg/day, orally), or the combination. Ifenprodil and sorafenib were purchased from Selleckchem (#54091) and LC laboratories (#S-8502), respectively. The drug treatment was started after grouping, and lasted for 21 days. Tumor volume and body weight of each mouse were then measured every two days. The estimated tumor volume served as a guide for determining the day when the mice were sacrificed to take actual measurements on the resected tumors. The mice were sacrificed, and the tumor tissues were collected at the end of treatment. Part of the tumor tissues was snap-frozen in liquid nitrogen for protein extraction and immunoblot analysis. Part of the tissues was fixed in 4% paraformaldehyde (PFA) at room temperature for histological staining. The remaining tissues were dissociated into single cells for the limiting dilution spheroid formation assay. To evaluate the tumor-initiating and self-renewal abilities of the drug-treated tumors derived from MHCC97L cells and PDX1 in vivo, limiting dilution and serial transplantation assays were performed. 500, 1,000, 5,000, 10,000, 50,000 cells dissociated from the residual tumor tissues were injected subcutaneously into either flank of 4-5 week-old mice. No drug treatment was further applied. The mice were checked every two days, and tumor incidence and latency were recorded. To evaluate HCC metastases, 4×10⁴ RIL-175 cells were injected through tail vein into 6- to 8-week-old male nude mice. Drug administration began at day 5 post-injection, and daily treatment with DMSO, ifenprodil (20 mg/kg/day, intraperitoneally), sorafenib (28 mg/kg/day, orally), or the combination was applied for 10 days before resection of lung tumors. At five and twelve days after the tail-vein injection, the mice were administered 100 mg/kg D-luciferin via peritoneal injection for bioluminescent imaging (using the PE IVIS Spectrum in vivo imaging system) to monitor the lung metastasis.

Colony Formation Assay

Lentivirus-transduced MHCC97L-Cas9 and Hep3B-Cas9 cells expressing the sgRNAs were seeded onto one well of a 6-well plate. Colonies were fixed by ice-cold methanol at −20° C. for 15 minutes and stained by crystal violet. Colony number and area were determined by ImageJ software.

Limiting Dilution Spheroid Formation Assay

Resuspended cells were cultured in DMEM/F12 (ThermoFisher Scientific) medium containing 0.25% methylcellulose (Sigma-Aldrich), 20 ng/ml EGF (Sigma-Aldrich), 10 ng/ml FGF (Sigma-Aldrich), 4 μg/ml insulin (Sigma-Aldrich), 1% B27 (ThermoFisher Scientific), and 1% antibiotic-antimycotic (ThermoFisher Scientific). Serial dilution of cells (5, 10, 20, 50, and 100 cells from the HCC cell lines and cells dissociated from the xenografts; 10, 20, 50, 100, 200 cells from the HCC organoids) were seeded onto a 96-well plate in 6 replicates for each dilution. Fresh medium was replenished every two days. The number of wells with spheroids formed were counted after a 10-day culture. The stem cell frequency was calculated using ELDA: Extreme Limiting Dilution Analysis (internet site bioinf.wehi.edu.au/software/elda/). In the data plots, the y-axis “fraction nonresponding” refers to the log proportion of number of cultures not responding to form spheroids, while the x-axis “dose (number of cells)” refers to the number of cells used in each culture. The slope of the line is the log-active cell fraction, representing the estimated active cell frequency in forming spheroids, and the dotted lines gives the 95% confidence interval. For example, “Vector control 1:13” indicates that the estimated active cell frequency in forming spheroids in the vector control is 1 out of 13.

Immunoblot Analysis

Cells or homogenized tissues collected from xenografts were lysed by 2X RIPA lysis buffer supplemented with protease inhibitor cocktail (ProBlock™ Gold Mammalian, Goldbio), PhosSTOP (Roche), 1 mM Na3VO4, and 1 mM NaF. Lysates were collected by scraping of the culture plate on ice, and then centrifuged at 15,000 r.p.m. for 15 minutes at 4° C. Supernatants were quantified using the Bradford assay (BioRad). Protein was denatured at 99° C. for 5 minutes before gel electrophoresis on an 8-15% polyacrylamide gel (Bio-Rad). Proteins were transferred to polyvinylidene difluoride membranes at 110V for 2 hours at 4° C. The primary antibodies used were anti-β-catenin (1:1,000; BD Biosciences, 610154), anti-Cyclin D1 (CCND1) (1:1,000; ab16663), anti-MMP-14 (1:1,000; ab51074), anti-PCNA (1:1,000; ab29), anti-TTK (1:1,000; ab11108), anti-MLKL (1:1,000; ab184718), anti-MLKL (phosphor S358) (1:200, ab187091) (Abcam), anti-Bcl-2 (C-2) (1:1,000, sc-7382), anti-c-Myc (9E10) (1:500, sc-40) (Santa Cruz), anti-Bad (1:1,000, #9292), anti-phospho-Bad (Ser112) (7E11) (1:1,000, #9296), anti-phospho-Bcl-2 (Ser70) (5H2) (1:1,000, #2827), anti-cleaved Caspase-3 (Asp175) (1:1,000, #9661), anti-Caspase-7 (1:1,000, #9492), anti-Caspase-9 (C9) (1:1,000, #9508), anti-CHOP (1:500; #2895), anti-eIF2a (1:1,000; #9722), anti-phopho-eIF2a (Ser51) (1:1,000; #3398), anti-GAPDH (1:5,000; #2118), anti-IRElU (14C10) (1:1,000; #3294), anti-LC3B (1:1,000; #2775), anti-MMP-7 (1:200; #71031), anti-PARP (1:1,000, #9542), anti-PERK (1:1,000; #3192), and anti-p21^{Cip1} (1:1,000; #2947) (Cell Signaling), anti-CDK2 (1:1,000; Bethyl Laboratories, #A301-812a), anti-IRE1 alpha (p Ser724) (1:1,000; Novus Biologicals, NB100-2323), anti-LGR5 (1:500; Origene, TA503316) and anti-p-actin (1:10,000; Sigma, A2228). The secondary antibodies used were horseradish peroxidase (HRP)-linked anti-mouse IgG (1:10,000; Cell Signaling, #7076) and HRP-linked anti-rabbit IgG (1:20,000; Cell Signaling, #7074). Membranes were developed by Clarity MAX™ Western ECL Substrate (Bio-Rad). The light emission was exposed and detected at the ChemiDoc™ Imaging System (Bio-Rad).

Immunohistochemistry Staining

Tumor tissues fixed in 4% PFA were sliced into sections. The paraffin sections were dewaxed in xylene, and then rehydrated in series of ethanol and washed in distilled water. Following the rehydration, the sections were heated for 15 minutes at 95° C. water bath in Dako Target Retrieval Solution (citrate buffer, pH 6.0, Dako) to restore antigenicity and enhance antibody-epitope binding. The sections were then incubated with 3% hydrogen peroxidase (H₂O₂) for 30 minutes and washed with PBS three times for 5 minutes. Primary antibodies were diluted in Antibody Diluent (Dako) and added to the slides and incubated overnight at 4° C. within a moist chamber. The primary antibodies used were anti-LGR5 (OTI2A2) (1:150, Origene, TA503316), anti-Axin2 (1:300, Abcam, ab109307), anti-IRE1 alpha (p Ser724) (1:300, Novus Biologicals, NB100-2323), anti-PECAM-1 (CD31) (1:100, Santa Cruz Biotechnology, sc-1506), and anti-p21^{cip1} (1:50, Cell Signaling #2947). The sections were again washed by PBS three times and incubated with Peroxidase Labeled Polymer rabbit/mouse (Dako, K4002/K4000) for 30 minutes at room temperature the day after. Color detection was performed by DAB+Substrate-Chromogen System (Dako) was added and subsequently the nuclei were counterstained with hematoxylin. The unbound stain was removed with acid ethanol. The sections were rehydrated and mounted with mounting medium and observed under a microscope. Quantification of staining densities of the immunohistochemistry images was performed using Image J. Five random fields were selected for quantification. To quantify the number of CD31-positive vessels, each positive endothelial cell cluster within each image was counted as an individual vessel in addition to the morphologically identifiable vessels with a lumen. Three random fields were selected for quantification.

Luciferase Assay

Cells were transfected with a firefly luciferase-based plasmid (TOPFlash or p5xATF6-GL3 (Addgene, 11976) (Wang, et al., J. Biol Chem. (2000), 275(35):27013-20) and a renilla-luciferase plasmid (200:1). An equal number of transfected cells were re-plated and treated with DMSO, ifenprodil alone, sorafenib alone, or combination of ifenprodil and sorafenib for 24 hours. The signal of luciferase was detected by Dual-Luciferase® Reporter Assay System (Promega) according to the manufacturer's instructions. Relative luciferase signal was measured and calculated using the formula: (signal of firefly luciferase/signal from renilla luciferase)/the averaged intensity from DMSO-treated group.

Dissociated cells from organoids were infected with a lentiviral-based TOPFlash plasmid (Addgene, 24307) for 6 hours. This plasmid contains a 7 X Tcf-firefly luciferase-based reporter cassette followed by another cassette driving mCherry expression under a constitutive promoter (Fuerer and Nusse, PLoS One (2010), 5(2): e9370). After a 3-day expansion in Matrigel, cells were re-plated and cultured for another 3 days to form organoids. The organoids were then treated with DMSO, ifenprodil alone, sorafenib alone, or combination of ifenprodil and sorafenib for 3 days. Cells were collected and analyzed using the Dual-Luciferase® Reporter Assay System as described above. Together with 7-AAD (7-amino-actinomycin D) staining, the percentage of mCherry-positive live cells within the organoids were also quantified using flow cytometry and was used for normalization in the luciferase assay.

RNA-Sequencing

3×10⁵ MHCC97L cells were seeded onto a 10 -cm dish. 10 μM ifenprodil, 5 μM sorafenib, or both were added to the cells 1 day after seeding, and the control group was treated with DMSO (Sigma-Aldrich). After 24 hours of drug treatment, RNA was extracted using the MiniBEST Universal RNA Extraction Kit (TaKaRa) following the manufacturer's protocol. The final product was eluted in DEPC-treated H₂O (ThermoFisher Scientific). RNA-seq experiments were performed at the Centre for Genomic Sciences (LKS Faculty of Medicine, The University of Hong Kong). The Illumina adaptors of the paired-end raw sequence reads were trimmed by Trimmomatic 0.39. The STAR aligner version 2.7 was used to align the sequence reads to the human genome (GRCh38.p13, Ensembl release 99) and quantify read counts for each Ensembl gene feature, which generated the raw count gene matrix. Differential expression analysis was performed in R (v 3.6.3) using DESeq2 (1.26.0) with the raw count matrix as input. Three biological replicates each for the ifenprodil-, sorafenib-, and ifenprodil+sorafenib combination-treated groups are compared against the DMSO-treated negative control group. Differentially expressed genes (DEGs) were identified as genes with the absolute values of its shrunken log₂ fold-changes bigger than 1 and a false discovery rate smaller than 0.05. Gene annotation was done using the bioMaRt (2.42.1) package with Ensembl Human genes (GRCh38.p13) dataset. Gene ontology enrichment analysis was performed on up/down regulated DEGs retrieved from either ifenprodil, sorafenib, or ifenprodil+sorafenib treatment using string v11.0 (internet site: string-db.org/). Top hits Gene ontologies (biological processes) were identified as GO enriched with a false discovery rate less than 0.01 and contain more than 40 observations for over-expressions and down-regulations of DEGs in total across all treatments. More specific GO terms containing fewer than 3000 genes and fewer than 300 observations across all treatments were then focused on.

Quantitative-PCR

RNA was extracted from MHCC97L cells treated with 10 μM ifenprodil, 5 μM sorafenib, or both, for 24 hours using the MiniBEST Universal RNA Extraction Kit (TaKaRa) following the manufacturer's protocol. cDNA was synthetized using PrimeScript RT Reagent Kit (TaKaRa), based on random hexamers according to the manufacturer's protocol. Standard quantitative PCR was run on Roche LightCycler480 II Real-time PCR system using TB Green® Premix Ex Taq™ (TaKaRa) following the manufacturer's protocol. The forward (F) and reverse (R) primers used were:

(1) ABCB1:
F:
(SEQ ID NO: 106)
AAA TTG GCT TGA CAA GTT GTA TAT GG,
and
R:
(SEQ ID NO: 107)
CAC CAG CAT CAT GAG AGG AAG;
(2) ABCC1:
F:
(SEQ ID NO: 108)
CTCCTCCTATAGTGGGGACATCAG,
and
R:
(SEQ ID NO: 109)
GTAGTCCCAGTACACGGAAAG;
(3) ABCC2:
F:
(SEQ ID NO: 110)
ATG CAG CCT CCA TAA CCA TGA,
and
R:
(SEQ ID NO: 111)
CTT CGT CTT CCT TCA GGC TAT TCA;
(4) ABCG2:
F:
(SEQ ID NO: 112)
TCA TCA GCC TCG ATA TTC CAT CT,
and
R:
(SEQ ID NO: 113)
GGC CCG TGG AAC ATA AGT CTT;
and
(5) NESTIN:
F:
(SEQ ID NO: 114)
CTG CGG GCT ACT GAA AAG TT,
and
R:
(SEQ ID NO: 115)
AGG CTG AGG GAC ATC TTG AG

Connectivity Map Analysis

Connectivity Map analysis was performed on CLUE (internet site: clue.io) (Subramanian et al. (2017) Cell 171: pages 1437-52 e17). For predicting the drugs that can potentially target CSC, two gene signatures, CD133 and EpCAM CSC signature, were analyzed. The top 150 upregulated and top 150 downregulated genes (or all downregulated genes (123 genes) for CD133 signature) from sorted CD133+liver CSCs versus CD133-differentiated subsets or sorted EpCAM+liver CSCs versus EpCAM-differentiated subsets were selected after ranking by log₂ fold change. For comparing the connectivity of sorafenib, ifenprodil and sorafenib plus ifenprodil treatment with other drugs, the top 150 upregulated and top 150 downregulated genes of each drug compared to DMSO control were selected as signatures. The gene signatures were queried against the L1000 gene expression with database 1.0. The gene signature of sorafenib plus ifenprodil were also inputted into L1000CDS2 (Duan, et al., NPJ Systems Biology and Applications (2016), 2(16015), DOI: https://doi.org/10.1038/npjsba.2016.15) for comparing the similarity with other drug combinations, among which none of them showed high connectivity.

Clinical Data Set

The Cancer Genome Atlas (TCGA)—Liver Hepatocellular Carcinoma (LIHC) dataset was downloaded from the Xenabrowser (internet site: xena.ucsc.edu/, UCSC, USA). Gene expression was compared in non-tumor liver samples and liver tumor samples, and its association with overall survival.

Quantification and Statistical Analysis

Data analyses were performed using GraphPad Prism 7 software (GraphPad Software). Data expressed are mean±SD, biological replicates are specified for each experiment in the figure legends. Two-tailed independent Student's t-test was applied to analyze the statistical difference between any two groups. The one-way ANOVA and subsequent Tukey's post hoc test was used for multiple groups comparison. Survival curves were plotted by the Kaplan-Meier method and the statistical P value was generated by the Cox-Mantel log-rank test. ****: P<0.0001; ***: P<0.001; **: P<0.01; *: P<0.05.

### Results

To identify combinatorial therapeutic targets for HCC, the CombiGEM-CRISPR v2.0-based screening strategy was adopted (Wong et al. (2016) Annual Rev Genet 50: pages 515-538; Wong et al. (2015) Nat Biotechnol 33(9): pages 952-61; Tong et al. (2018) J Hepatol 69(4): pages 826-839) (FIG. 1A). In the screening library, a set of existing genes or potential drug targets for suppressing HCC growth were covered, as well as those that are up-regulated in two liver CSC subsets and have matching drugs being available for targeting them readily. Data mining was performed in search of genes that are up-regulated in liver CSCs, using an in-house transcriptome data comparing the sorted CD133+liver cancer stem cells (CSCs) versus CD133⁻ differentiated subsets (Ma et al. (2010) Cell Stem Cell 7(6): pages 694-707; Tong et al. (2015) Stem Cell Reports 5(1): pages 45-59), and a publicly available transcriptome dataset comparing sorted EpCAM⁺ liver CSCs vs. EpCAM-differentiated subsets (GSE5975) (Yamashita et al. (2009) Gastroenterology 136(3): pages 1012-1024). Based on the above selection criteria, 27 genes were shortlisted to be included in the screening library (Table 3) Three additional genes that were previously identified to be preferentially overexpressed and activated in tumorigenic CD133+liver CSCs were also included (Tong et al. (2018) J Hepatol 69(4): pages 826-839; Ma et al. (2010) Cell Stem Cell 7(6): pages 694-707; Tong et al. (2015) Stem Cell Reports 5(1): pages 45-59) (Table 3). Three single guide RNAs (sgRNAs) per gene were designed, which have high on-target scores (i.e., at least 0.60 based on the Azimuth 2.0 model (Doench et al. (2016) Nat Biotechnol 34(2): pages 184-191) and selected three non-targeting control sgRNAs from the GeCKOv2 library (Shalem et al. (2014) Science 343(6166): pages 84-87) that do not have on-target loci in the human genome for library construction (Table 2). A high coverage (98.7%) barcoded pairwise sgRNA library was assembled with 93×93 sgRNAs (i.e., 8,649 total combinations).

A pooled screen was conducted to isolate pairwise sgRNAs that modulate growth of MHCC97L HCC cells stably expressing Streptococcus pyogenes Cas9. Barcode abundances between day 7 and day 0 (baseline) groups were compared to yield log₂ values as a measure of cell growth. Based on the genetic screen data, 11 gene combinations had at least two sets of sgRNA combinations with mean log₂ ratios of <−0.51 (with at least 30% fewer barcode counts in day 7—versus day 0—cultured cells) and P values of <0.05 based on results obtained from two biological replicates (FIG. 1E; Table 3). The resulting hit list contained gene combinations (FLT4+PDGFRA and FLT4+FGFR2) that encode the protein targets of first line multi kinase inhibitors sorafenib and lenvatinib for HCC treatment, as well as the FGFR3+ANXA3 combination that can be targeted by using sorafenib plus an anti-ANXA3 neutralizing antibody and was shown to inhibit HCC growth (Tong et al., 2018). This result highlights the relevance of the screen to isolate anti-cancer therapeutic targets. The screen also uncovered new druggable combinations that significantly reduce MHCC97L growth. Among those, NMDAR1+FLT4 and NMDAR1+FGFR3 were identified as the top hits to be characterized in this study, because four independent sets of sgRNA combinations targeting those genes gave a mean log₂ ratios of <−0.51 at P<0.05 and their gene products can be inhibited by using same matching drug pairs. Further analysis with The Cancer Genome Atlas (TCGA)—Liver Hepatocellular Carcinoma (LIHC) dataset found NMDAR1 expression in HCC tumor to be higher than that in non-tumor liver tissues (FIGS. 1J and 1K) and patients with low NMDAR1 expression showed better overall survival (FIG. 1L), highlighting its potential clinical relevance.

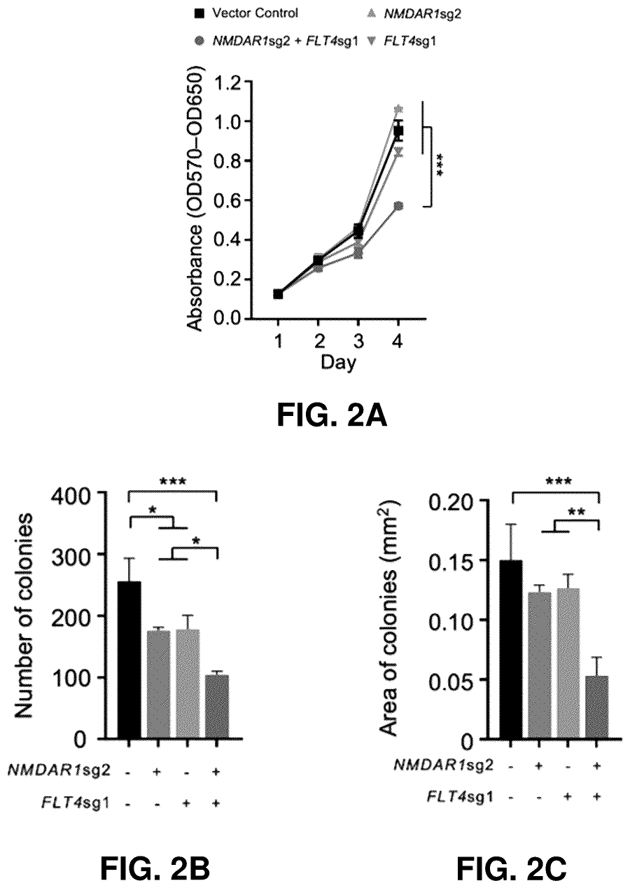

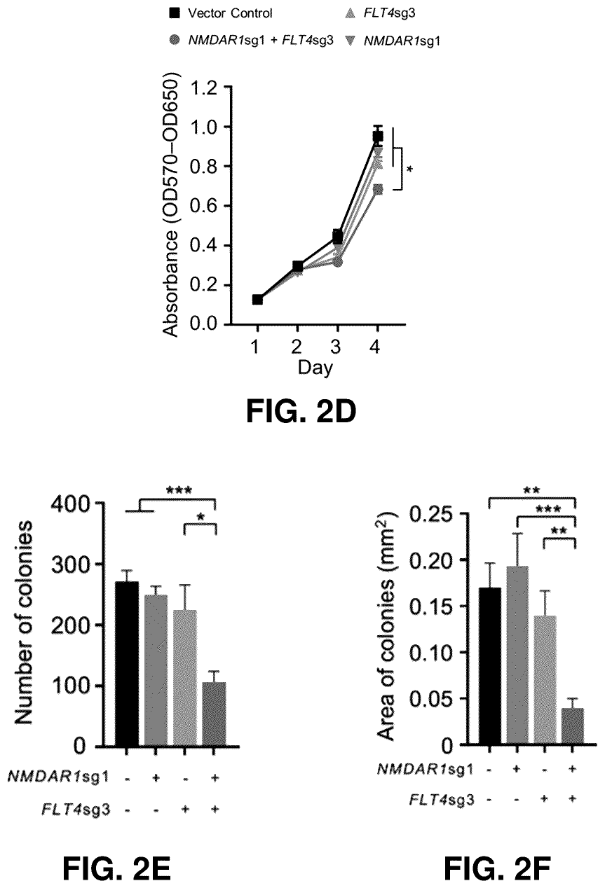

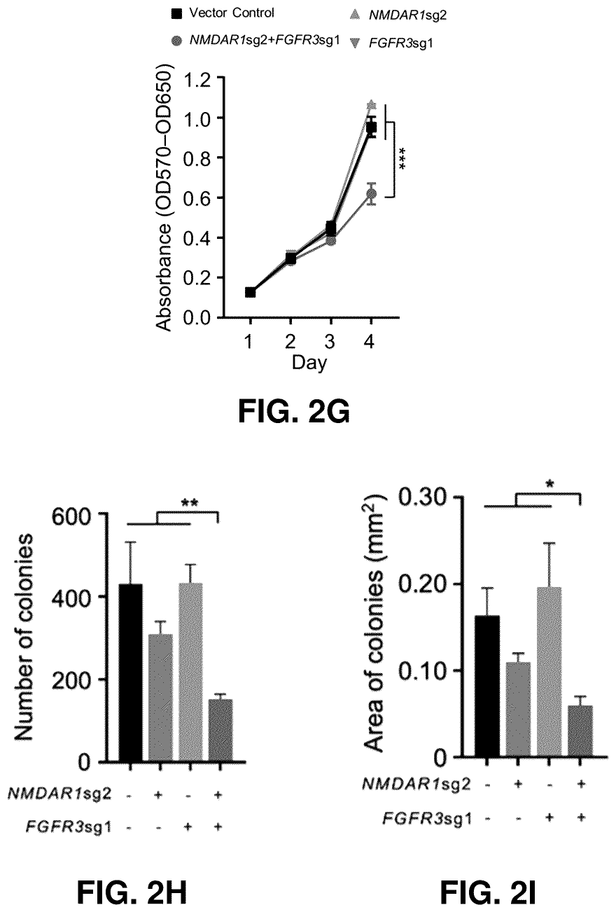

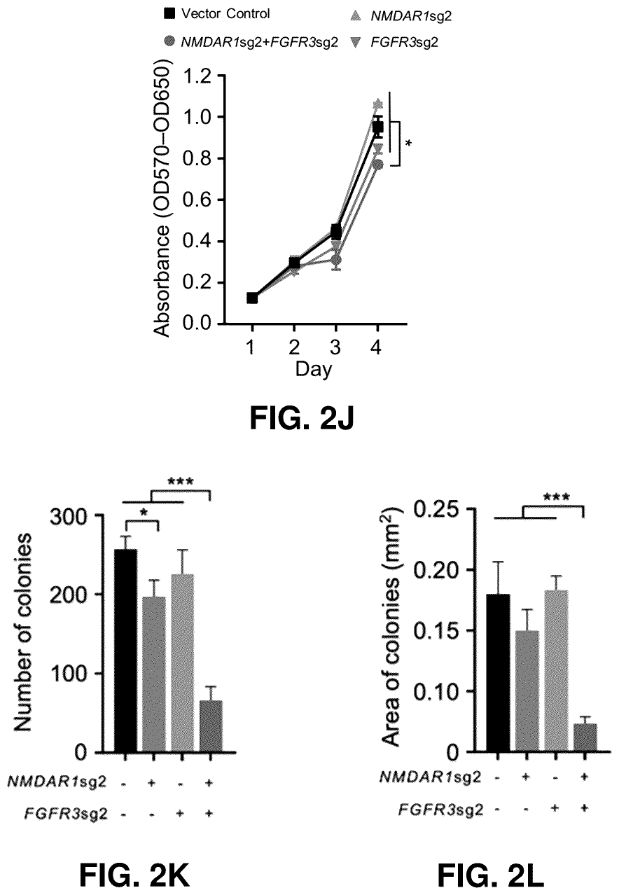

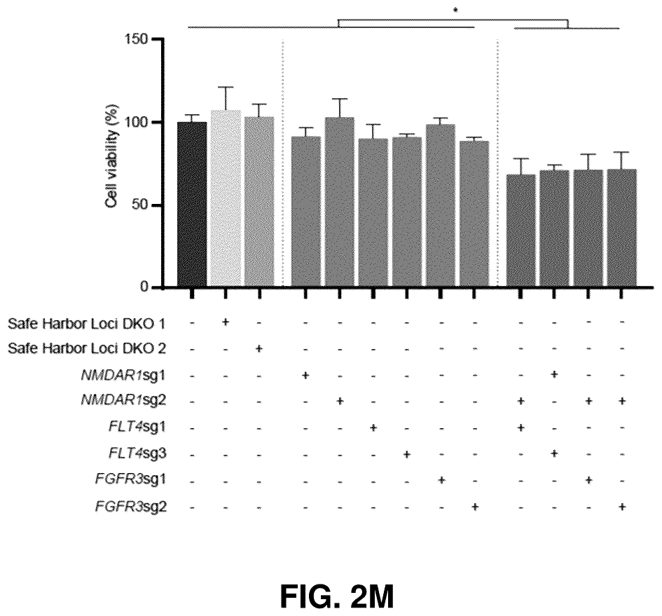

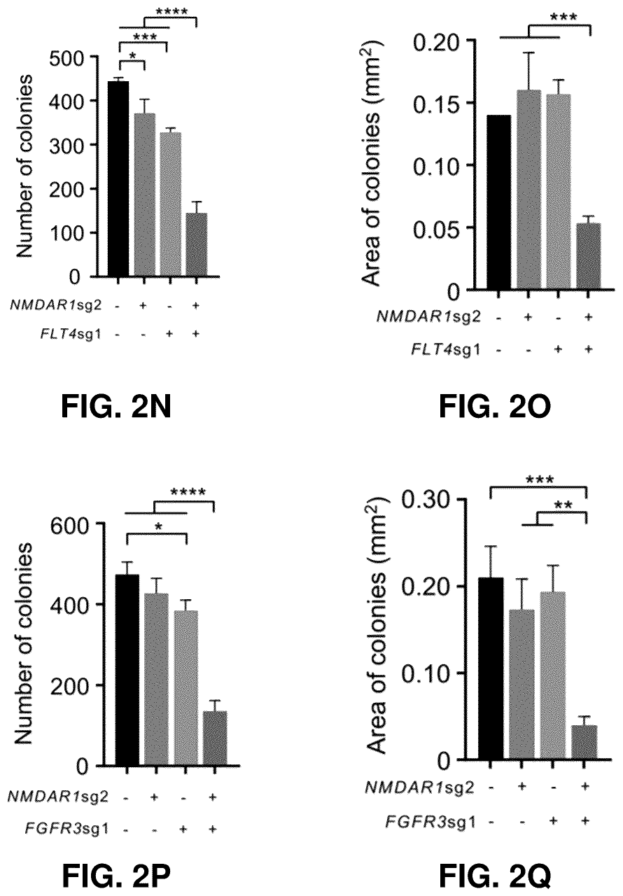

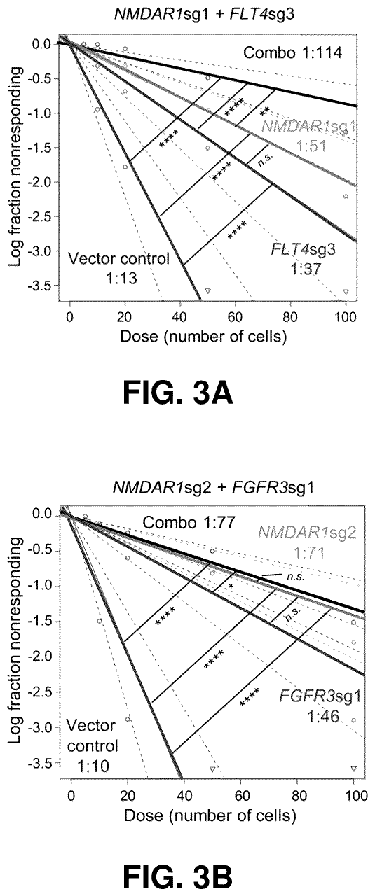

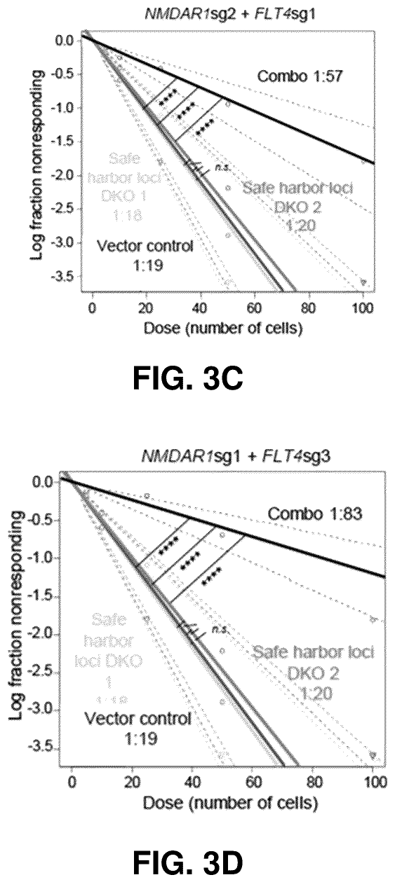

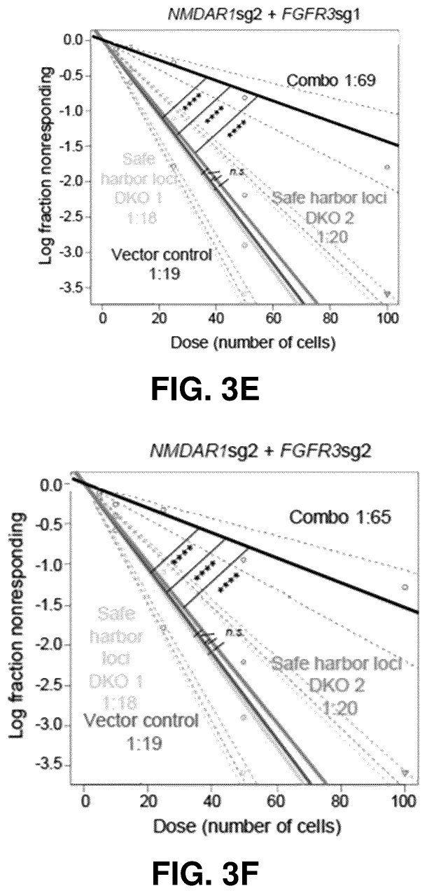

Using individual non-pooled cell viability and colony formation assays, the growth inhibition effects on MHCC97L cells by the four sgRNA combinations for NMDAR1+FLT4 or FGFR3 were separately validated (FIGS. 2A-2L). Little or much less inhibition was observed when those genes were genetically ablated (FIGS. 2A-2L). The growth inhibition effects brought by the sgRNA combinations were not due to excessive double-strand DNA breaks, because simultaneous targeting of two safe harbor loci did not inhibit growth (FIG. 2M). The DNA copy numbers of NMDAR1, FLT4, and FGFR3 loci were also not amplified in MHCC97L's genome (Luo et al (2020), Research Square). Similar growth inhibition effects brough by the sgRNA combinations were also observed in Hep3B cells (FIGS. 2N-2Q). Since NMDAR1 was upregulated in both EpCAM+ and CD133+HCC cell subpopulations in the transcriptome analyses described above, the capability of these sgRNA combinations to suppress the self-renewal ability of tumor-initiating cells that fuels cancer cell growth was evaluated. Using in vitro limiting dilution spheroid formation assays, it was demonstrated that the genetic co-ablation of NMDAR1+FLT4 and NMDAR1+FGFR3 substantially reduced the ability of MHCC97L to assemble tumor spheres, albeit that the effect brought by NMDAR1 ablation was not further enhanced when combined with FGFR3 ablation (FIGS. 3A and 3B).

### Example 2: NMDAR Inhibitors Synergize with Sorafenib and Lenvatinib to Reduce Viability and Self-Renewal Ability of HCC Cells

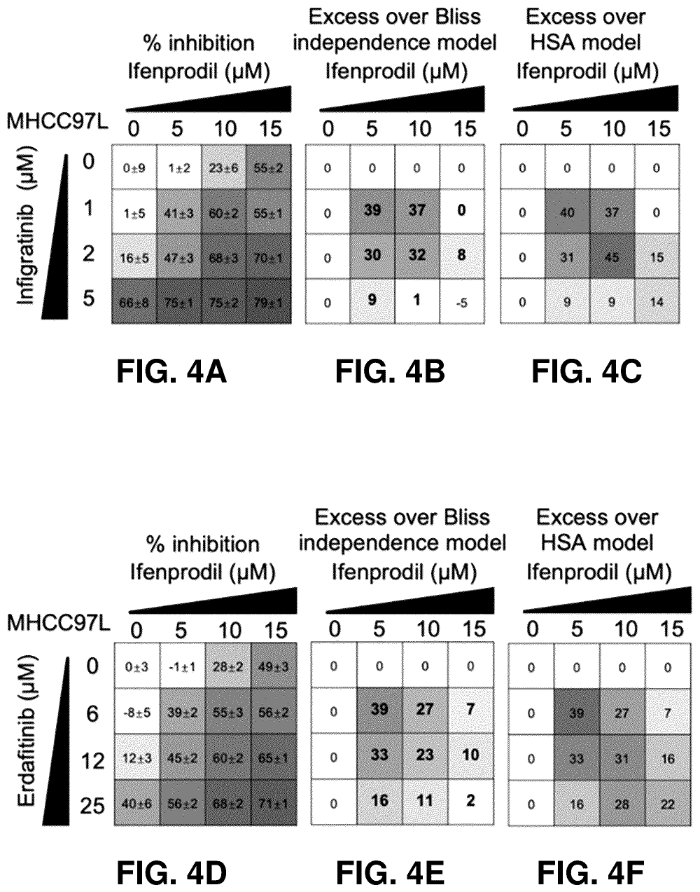

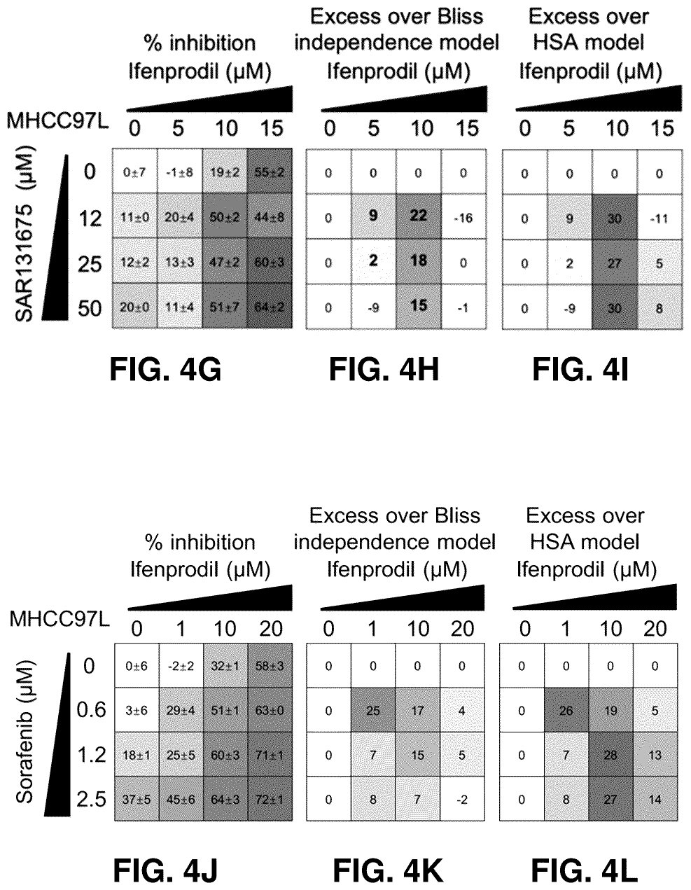

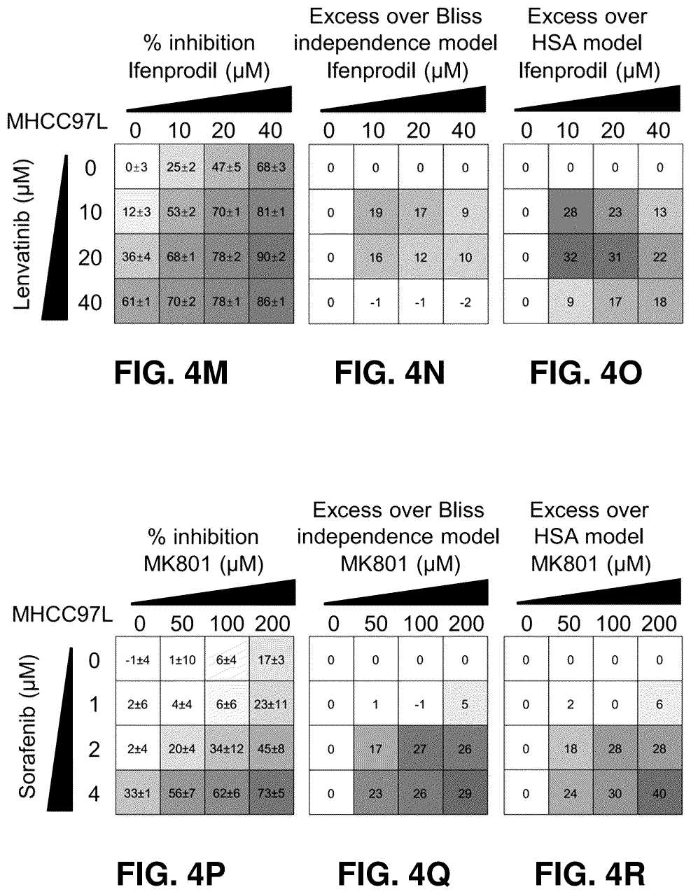

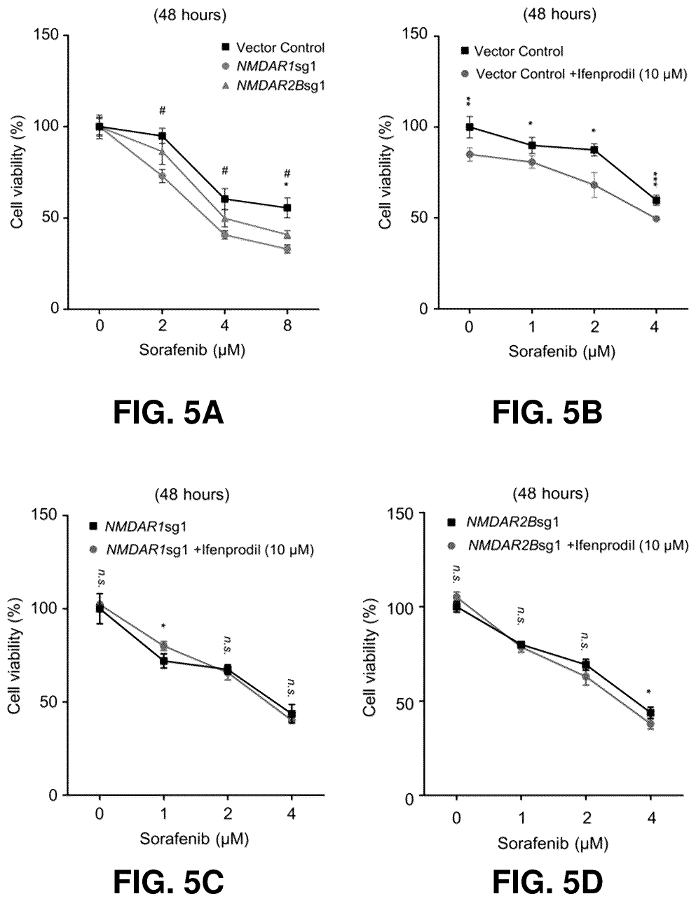

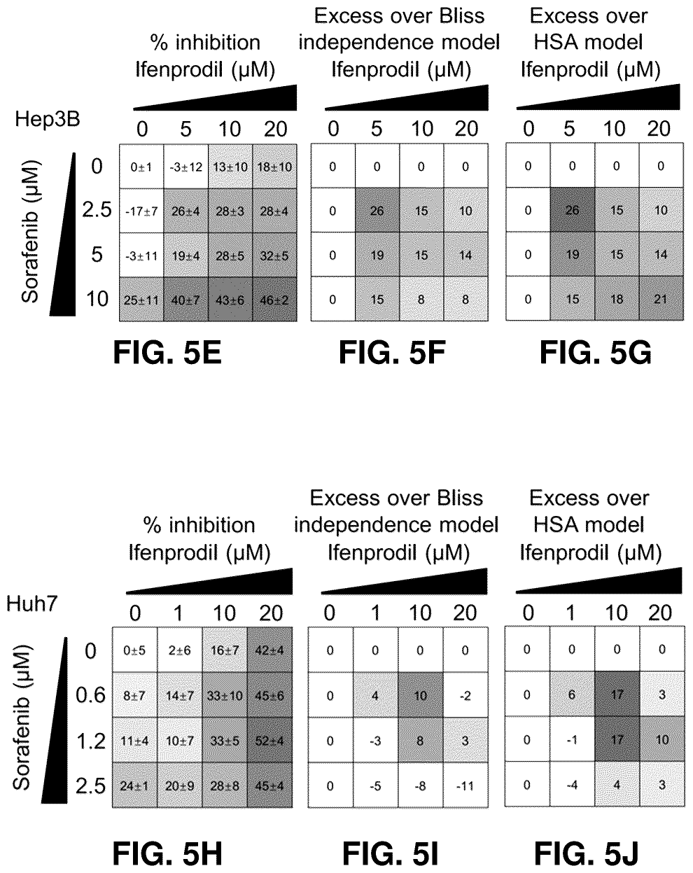

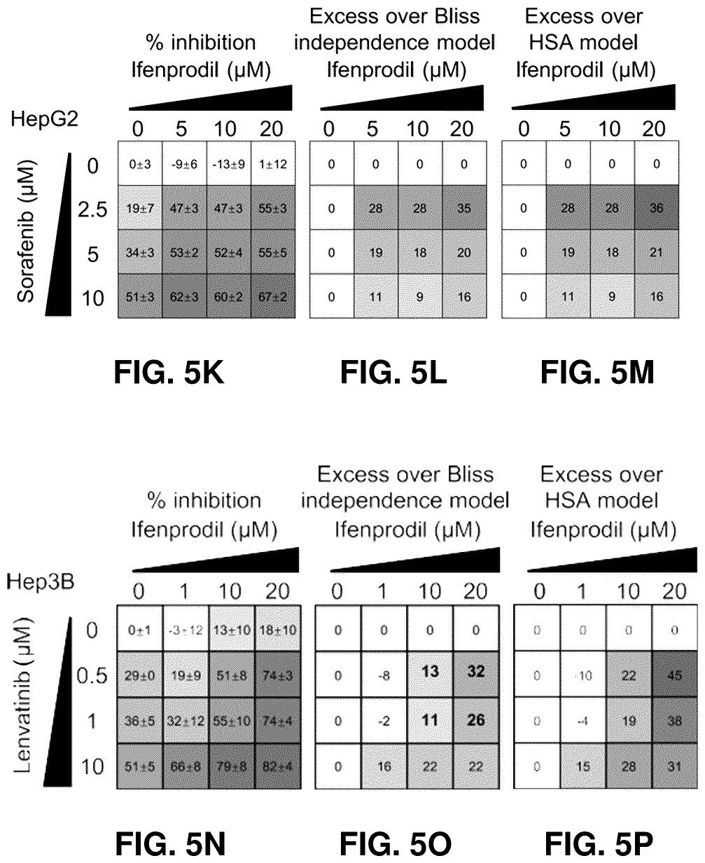

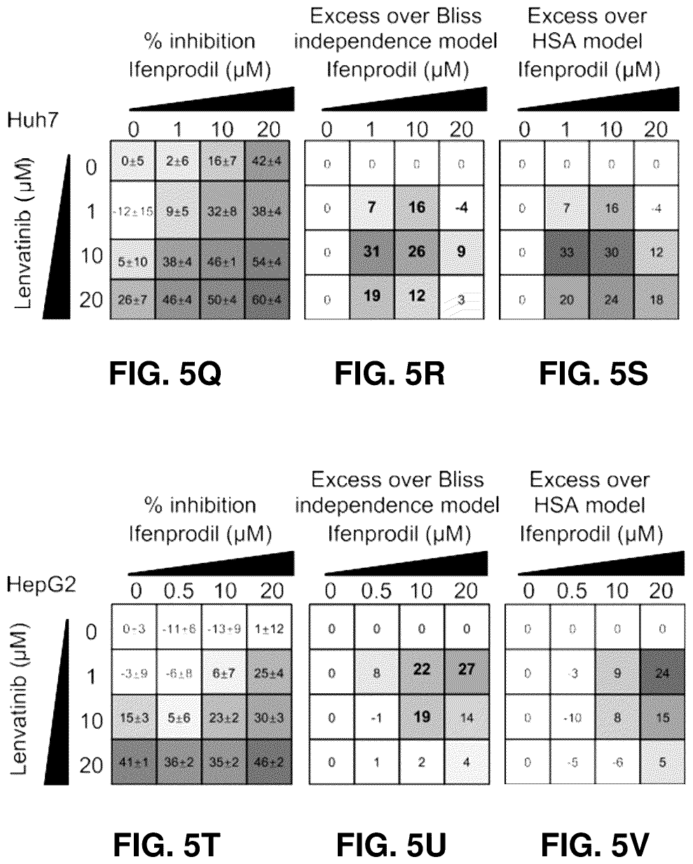

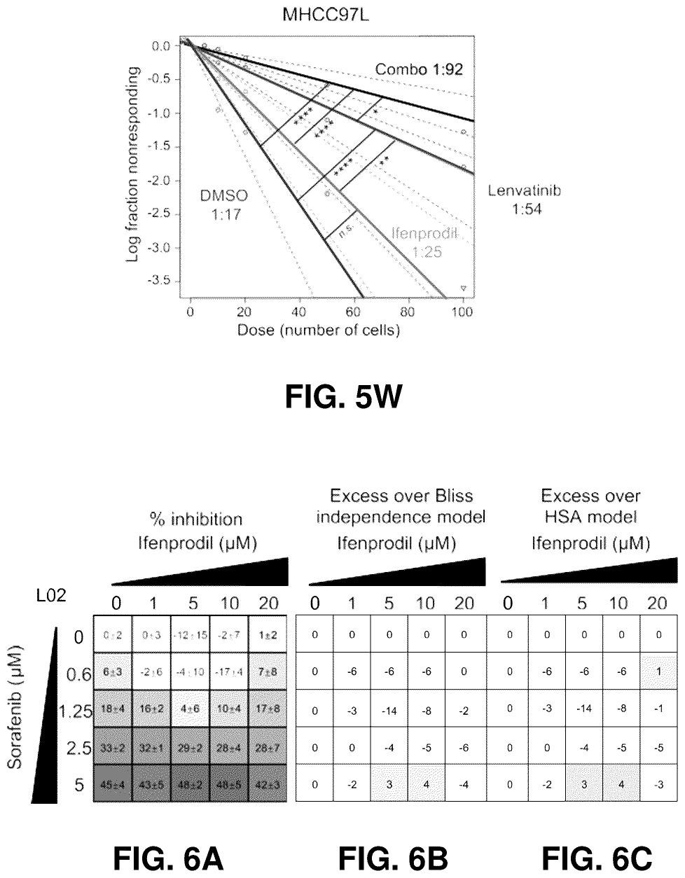

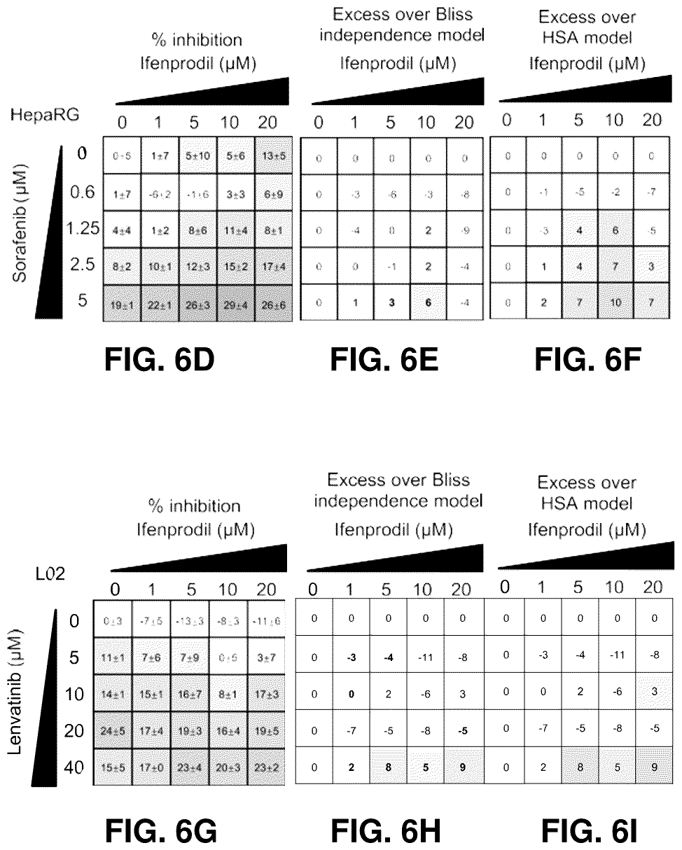

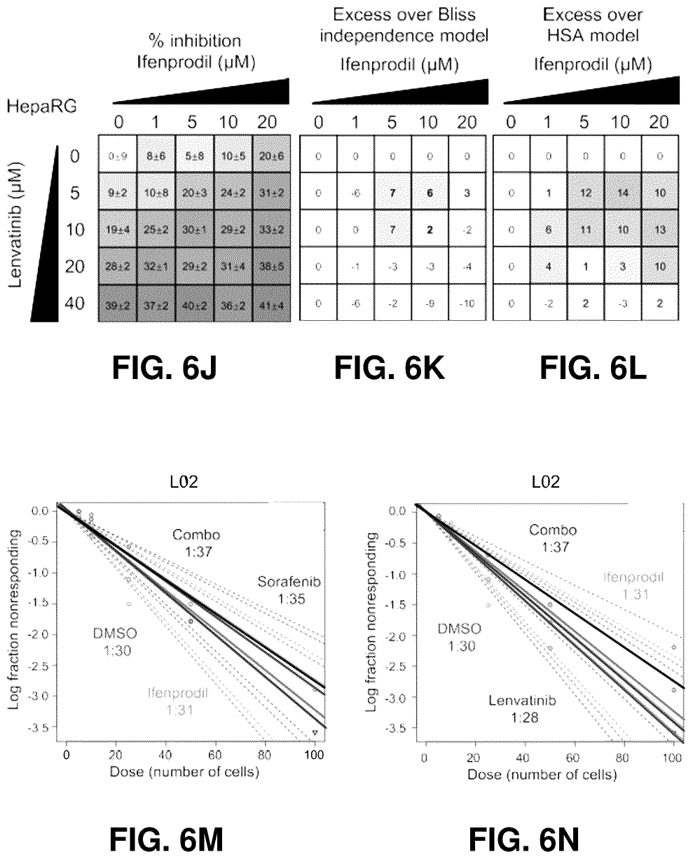

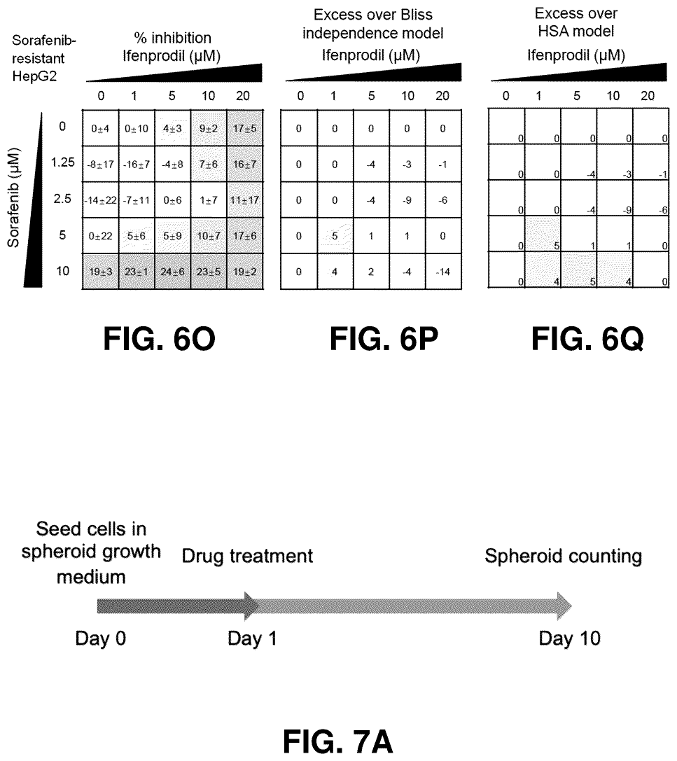

Genetic data led to the evaluation of the efficacy of targeting NMDAR1 in combination with FLT4 or FGFR3 for HCC treatment. The growth inhibition effects were examined by treating HCC cells with the matching inhibitor drugs. MK-801 and ifenprodil (IFEN) were used to inhibit NMDARs, while infigratinib (INF), erdafitinib (ERA), SAR131675 (SAR), sorafenib (SOR) and lenvatinib (LEN) were used to target FLT4 and/or FGFR3. In line with the genetic results described above, the results indicated that IFEN when combined with the FGFR inhibitors (INF and ERA) and the selective FLT4 inhibitor (SAR) greatly reduced the growth of the MHCC97L cells (FIGS. 4A-4I). IFEN and the broad-spectrum kinase inhibitors SOR/LEN also act synergistically to suppress the growth of MHCC97L cells (FIGS. 4J-40). Similar synergy was observed when MHCC97L cells were treated with MK-801 and SOR (FIGS. 4P-4R). MK-801 blocks all NMDAR subunits in a non-selective manner, which poses high toxicity and is less tolerated when used as a therapeutic agent (Chen and Lipton, 2006), while IFEN is more selective for NMDAR1/NMDAR2B subunits and has been used as a vasodilator in some countries including Japan and France with known safety history (Williams, (1993), Mol Pharmacol 44: pages 851-859). Similar to when NMDAR1 was genetically ablated, the growth inhibition was also observed when NMDAR2B was knocked out in sorafenib-treated MHCC97L cells (FIG. 5A). Minimal effects on growth inhibition when IFEN was added together with SOR to the NMDAR1- and NMDAR2B-ablated cells were observed (FIGS. 5B-5D), indicating that the IFEN-induced growth inhibition is likely NMDAR1/NMDAR2B-dependent. IFEN was used in the subsequent work. SOR/LEN were also selected for further examination because they are the first-line drugs used in HCC treatment. Synergistic effects by IFEN and SOR/LEN were observed for additional HCC cell lines such as Hep3B, Huh7, and HepG2 HCC cells (FIGS. 5E-5W), although the synergy observed for treatment with IFEN and SOR in Huh7 cells was relatively weaker. Hepatic L02 and HepaRG cells showed minimal synergistic effects to the IFEN and SOR co-treatment (FIGS. 6A-6N). Co-treatment of IFEN and SOR did not effectively inhibit growth of sorafenib-resistant HepG2 cells, and no synergy was detected, suggesting that additional anti-cancer effect brough by ifenprodil requires sorafenib's activity (FIGS. 6O-6Q). This result is consistent with the hypothesis that while synergism can provide a steep increment in efficacy when a second drug is added to the treatment regimen, it would also mean that the efficacy to the second drug would likely be decreased if cancer cells develop single-drug resistance to the first drug (Saputra et al (2018), Cancer Research, 78:2419-31).

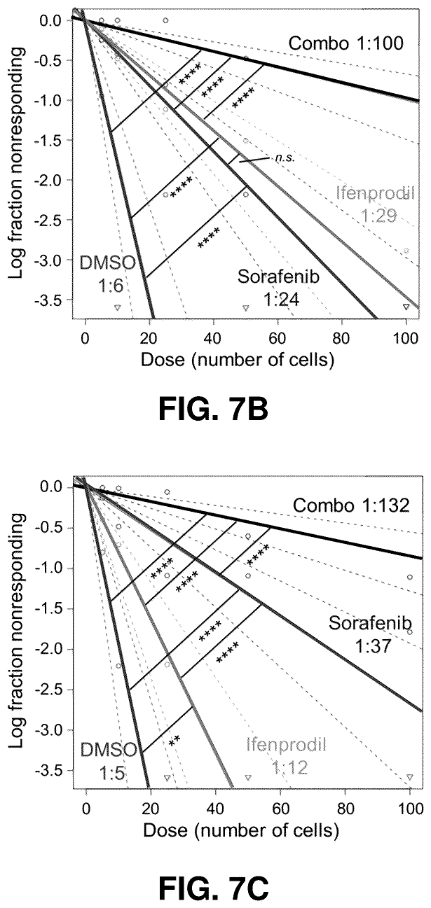

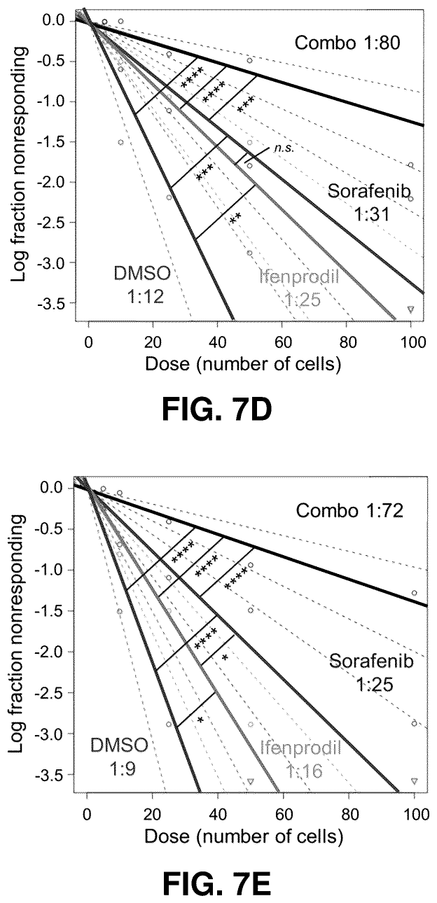

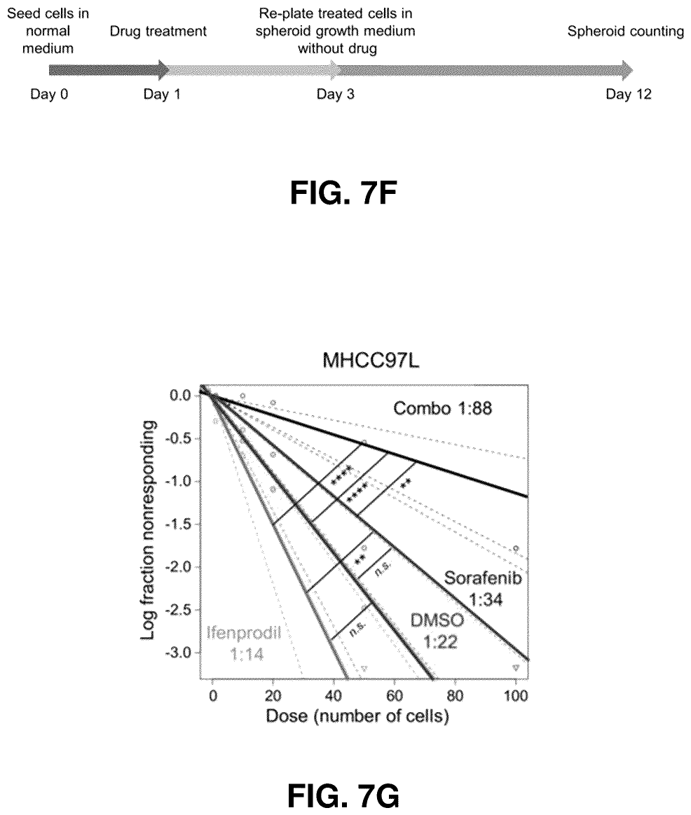

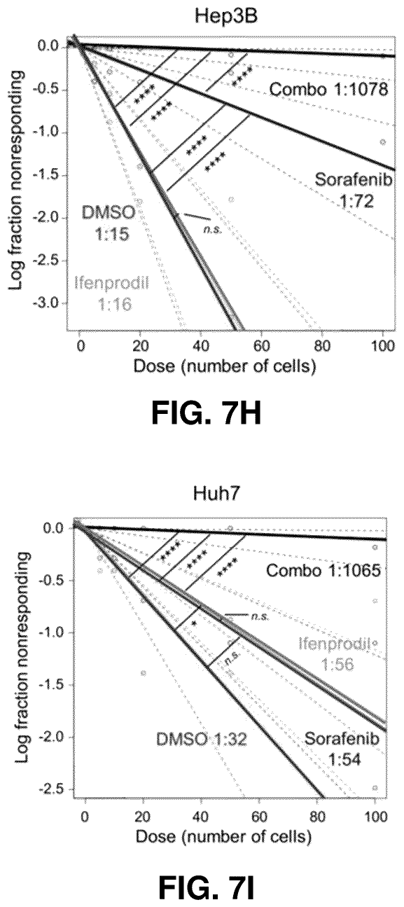

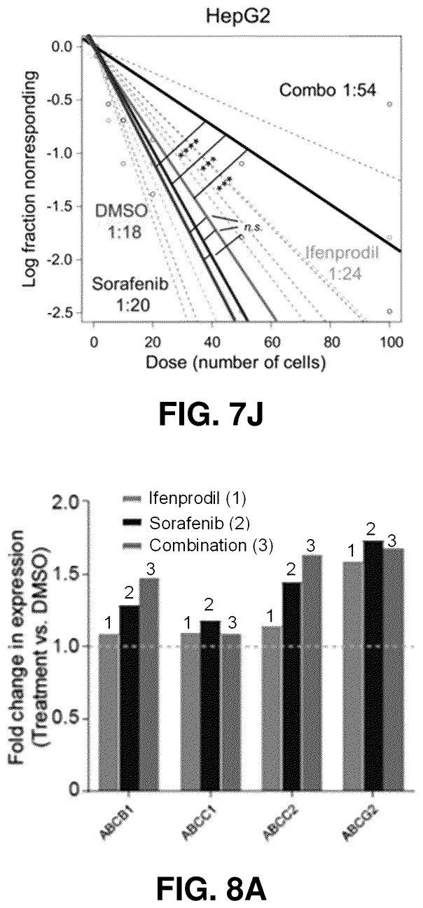

To measure the inhibition of HCC cell's self-renewal ability brought by the drug combination, in vitro limiting dilution spheroid formation assays were performed (FIG. 7A). Cells pre-seeded in spheroid growth medium were treated with ifenprodil and sorafenib. The number of tumor spheres formed was counted after 10 days. Co-treatment with IFEN and SOR or IFEN and LEN, but neither drug alone, markedly suppressed the ability of all four tested lines of HCC cells, but not L02, to form tumor spheres (FIGS. 7B-7E). Notably, similar levels of suppression were also observed when IFEN and SOR were only treated for 2 days to the HCC cell cultures prior to plating for spheroid formation assays (FIGS. 7F-7J), suggesting that the drug combination had eliminated the tumor-initiating cell population or added a molecular brake to abrogate the self-renewal capacity of the cells.

### Example 3: Co-Treatment of Ifenprodil and Sorafenib Induces ER Unfolded Protein Response, Downregulates WNT Signaling, and Results in G1-Phase Cell-Cycle Arrest in HCC Cells

The mechanism through which the IFEN+SOR combination inhibited HCC cell growth and self-renewal was explored. A previous study has reported that a competitive NMDAR antagonist 11 augments the cytotoxic action of sorafenib in murine HCC cells, and it may act by interfering with the lipid signaling pathway, reducing expression of multidrug resistance transporters (MDRs) and thereby increasing accumulation of sorafenib in cancer cells (Gynther et al. (2017) J Med Chem, 60: pages 9885-904).

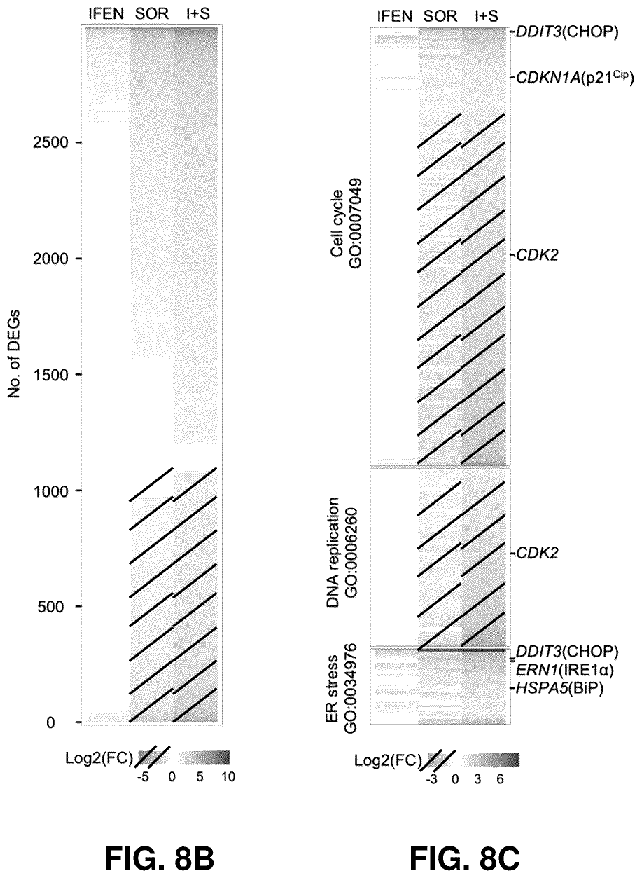

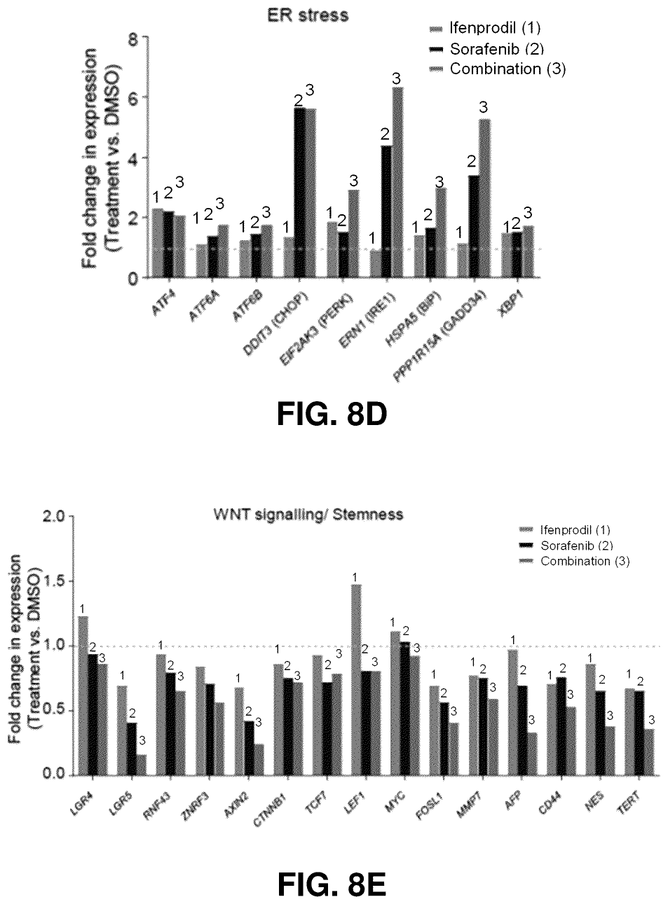

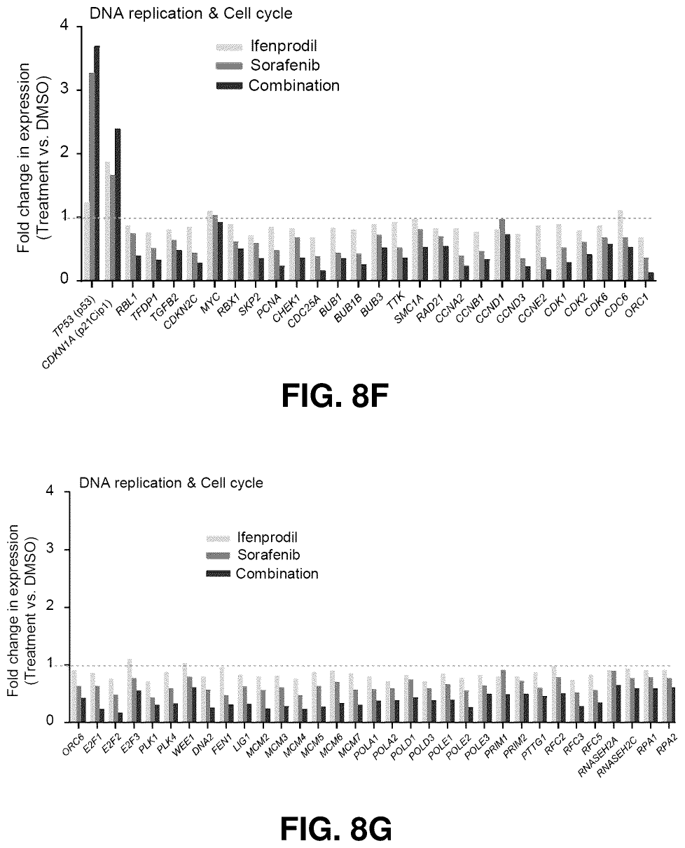

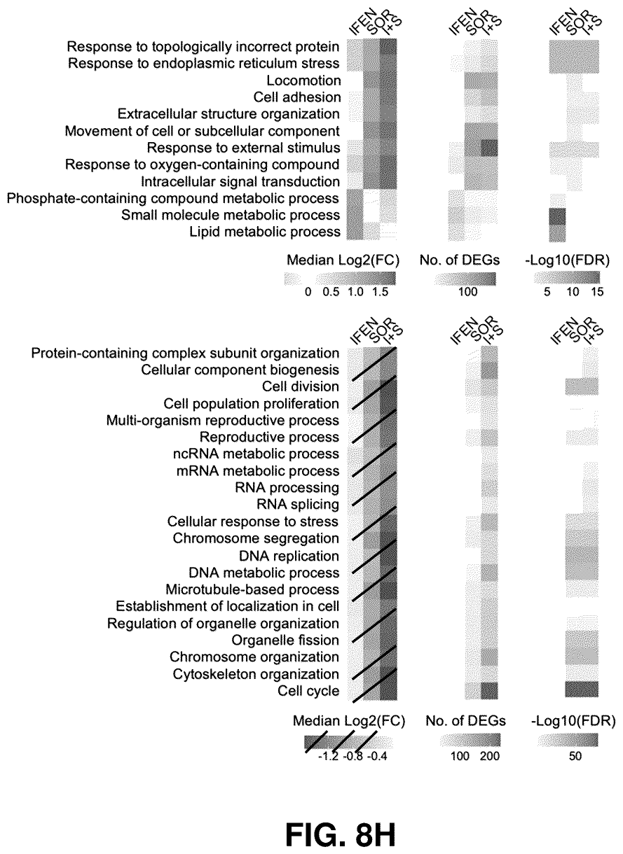

Unexpectedly, reduction of MDR transporter expressions in IFEN+SOR-treated MHCC97L cells was not observed (FIG. 8A). A transcriptome-wide analysis was performed using RNA-seq to identify differentially expressed genes and altered pathways in drug(s)-treated MHCC97L cells compared to non-drug treated cells (FIGS. 8B-8G). For example, notable genes for which differential changes in expression were observed include but are not limited to CDKN1A(p21Cip), CDK2, DDIT3 (CHOP), ERN1 (IRE1α), HSP5A (Bip). CDK1A and DDIT3 were significantly upregulated in SOR only treated cells, IFEN only—treated cells and SOR+IFEN treated cells compared to non-treated cells. CDK2 was significantly downregulated in SOR only treated cells, IFEN only-treated cells and SOR+IFEN treated cells compared to non-treated cells. Compared non-treated cells, ERN1 was significantly upregulated in SOR only treated cells and SOR+IFEN treated cells. However, no differences were observed in the expression of ERN1 in IFEN treated cells compared to non-treated cells (FIGS. 8B-8G). Mapping of the differentially expressed genes to Gene Ontology groups revealed that genes involved in endoplasmic reticulum (ER) unfolded protein response (UPR) are significantly upregulated, while many cell cycle- and DNA replication-related genes are downregulated, in cells treated with IFEN+SOR combination (FIG. 8H).

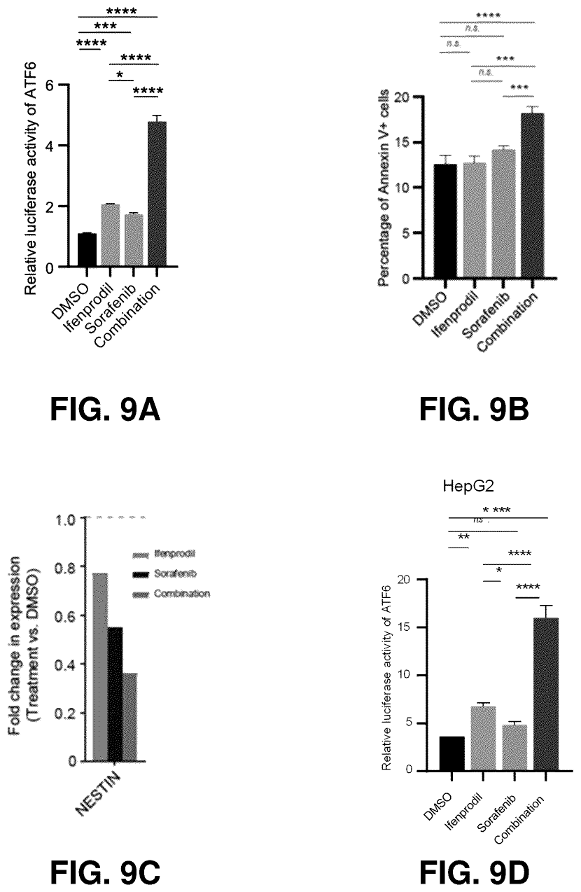

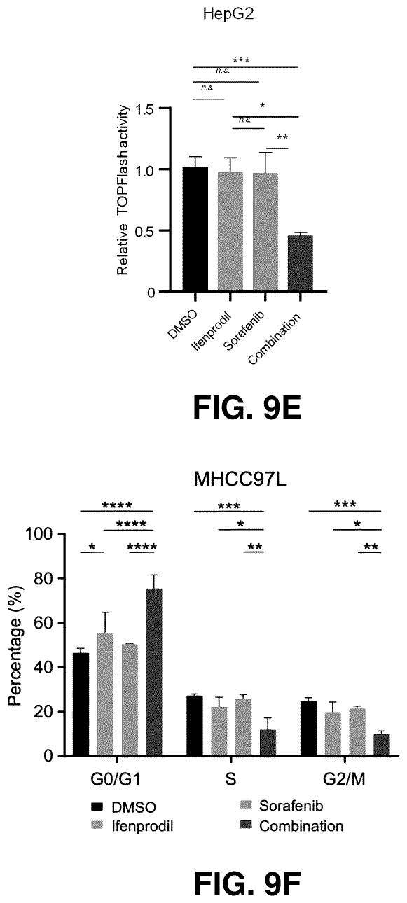

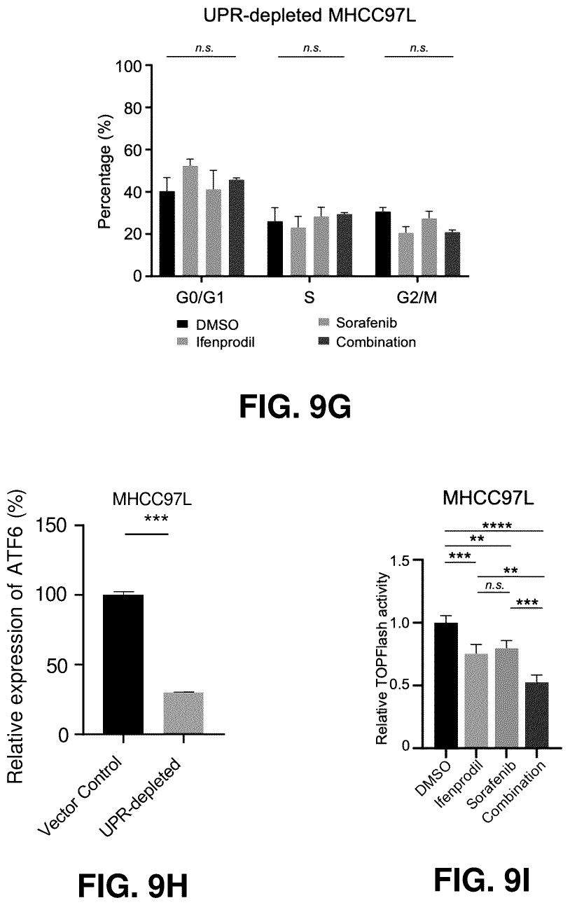

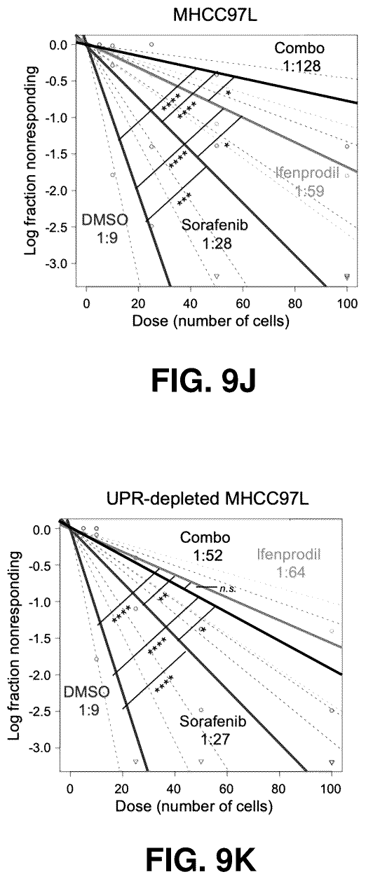

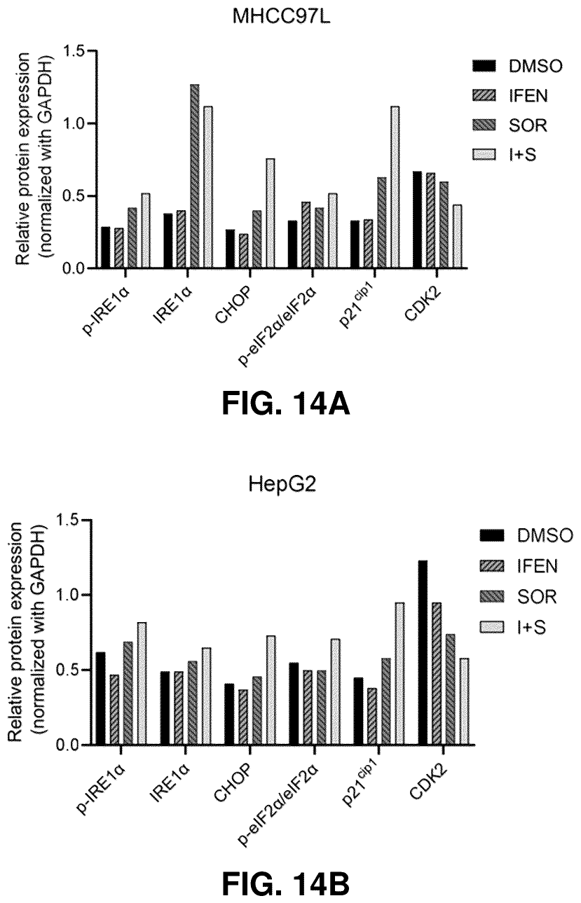

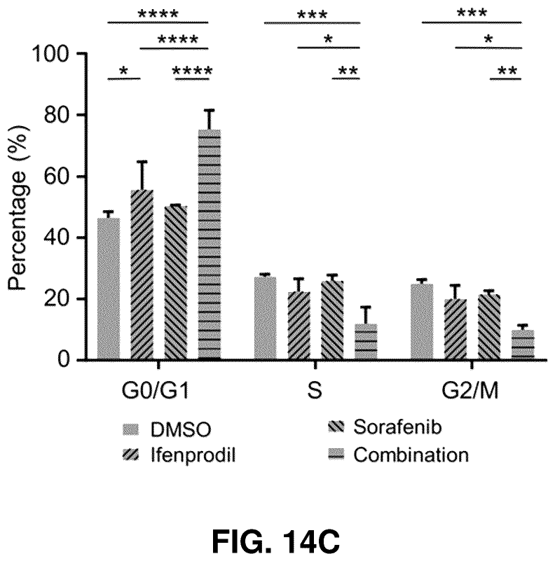

Upon ER stress, cells activate the unfolded protein response (UPR) (Hetz (2012) Nat Rev Mol Cell Biol 13: pages 89-102). Co-treatment of ifenprodil and sorafenib altered the expression of ER stress- and cell cycle-related proteins. MHCC97L cells were treated with DMSO, 10 μM ifenprodil, 5 μM sorafenib, or combination of ifenprodil and sorafenib, for 18 hours. HepG2 cells were treated with DMSO, 5 μM ifenprodil, 5 μM sorafenib, or combination of ifenprodil and sorafenib, for 48 hours. Western blot analysis was performed and confirmed that IFEN+SOR treatment activates UPR effectors, including phosphorylated IRE1-alpha and C/EBP Homologous Protein (CHOP), in both MHCC97L and HepG2 cells (FIG. 9A; FIGS. 14A and 14B). It was also confirmed that IFEN+SOR combination treatment greatly increased the expression of p21^{Cip1}, a regulator of cell-cycle progression at G1/S phase, which was accompanied by the downregulation of CDK2 (FIG. 9A) and other cell cycle and DNA replication-related proteins including PCNA and TTK, as well as the activation of apoptosis signaling pathway (FIGS. 9B and 9C). Similar results were also detected in another HCC cell line HepG2 treated with IFEN+SOR (FIG. 9H). Cell cycle analysis was performed, and G1-phase arrest was detected in MHCC97L cells treated with IFEN+SOR, but not with either IFEN or SOR alone (FIGS. 9F and 9G). To analyze whether IFEN+SOR treatment cause G1-phase arrest through UPR signaling, cells that were deficient in UPR sensing. Since UPR is an integrated ER stress-response pathway that is coordinated by three sensor proteins (i.e., IRE1-alpha, PERK, and ATF6), cells that were depleted of all three sensors were generated (FIG. 9H) as reported in previous studies (Adamson et al. (2016) Cell; 167: pages 1867-82 e21). It was found that G1-phase arrest and p21^{cip1} expression induced by IFEN+SOR treatment was rescued in these cells (FIGS. 9F-9H), indicating that the drug combination-induced G1-phase arrest depends on UPR.

Previous studies have shown that UPR induction is associated with the reduction of WNT- and stem cell-related gene expressions and cause the loss of cell stemness properties (Heijmans et al. (2013), Cell Rep 3:1128-39; Okamoto et al. (2020), J Biol Chem. 295:4591-603; Spaan et al. (2019), Cell Death Dis 10:490). In line with these findings, were detected in the RNA-seq data that WNT signaling-/stemness-related genes (including Lgr5 and Axin2), as well as other stem cell-related genes (including Nestin and Tert), were significantly downregulated in MHCC97L treated with IFEN+SOR (FIGS. 8E and 9C, TOPFlash assay was performed to report on the activation of WNT target genes. The results showed that WNT signaling was markedly downregulated in both MHCC97L and HepG2 cells treated with IFEN+SOR, when compared to those treated with vehicle or the single drugs alone (FIGS. 9E and 9I). It was further confirmed that decreased expressions of WNT target genes including Lgr5, c-Myc, CCND1, CTNNB1, and MMP-7 at the protein level. To directly evaluate whether the loss of cell stemness brought by IFEN+SOR treatment was UPR-dependent, limiting dilution assay was performed on the UPR-depleted MHCC97L cells and it was found that IFEN+SOR treatment were less effective in inhibiting the self-renewal of these cells (FIGS. 9J and 9K). The reduced protein expressions of the WNT target genes, including the cell sternness marker Lgr5, brought by the IFEN+SOR treatment were also relieved in the UPR-depleted MHCC97L cells. These results demonstrate that the drug combination-induced loss of cells stemness requires UPR. Collectively, the data demonstrates that IFEN and SOR synergizes to induce UPR, thereby suppressing the growth and self-renewal of HCC cells.

It was also confirmed that IFEN+SOR combination treatment greatly increased the expression of p21^{cip1}, a regulator of cell-cycle progression at G1/S phase, which was accompanied by the downregulation of its downstream effector Cdk2 in the HCC cells. G1-phase arrest was detected in the cells treated with IFEN+SOR, but not with either IFEN or SOR alone (FIG. 14C). Together, these results indicate that ifenprodil and sorafenib synergized to induce cell-cycle arrest and reduce WNT signaling, which could contribute to the suppression of growth and self-renewal observed in the drug combination-treated HCC cells.

### Example 4: Ifenprodil Treatment Enhances the Efficacy of Sorafenib in HCC Patient-Derived Organoids and 205 Xenograft Models

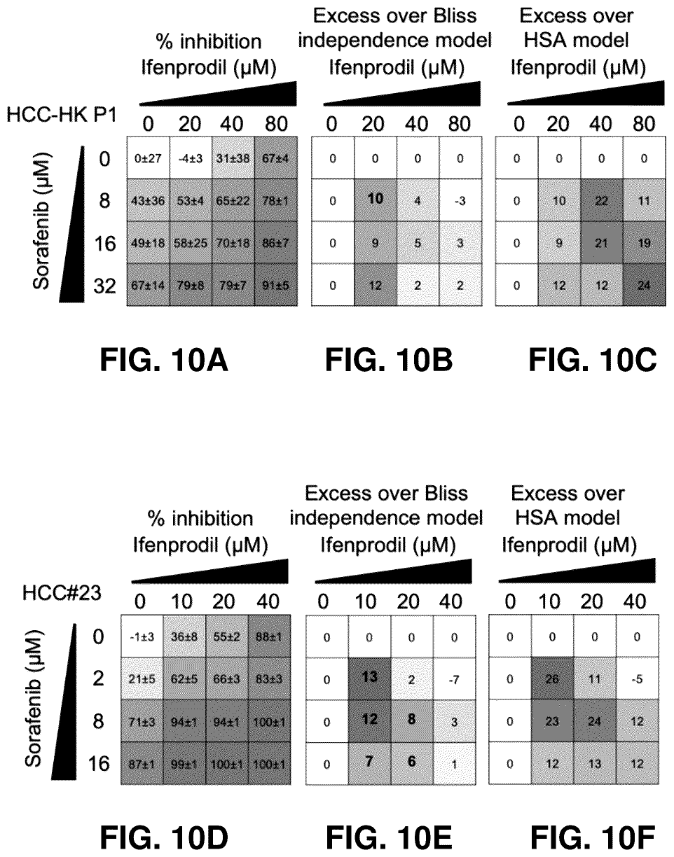

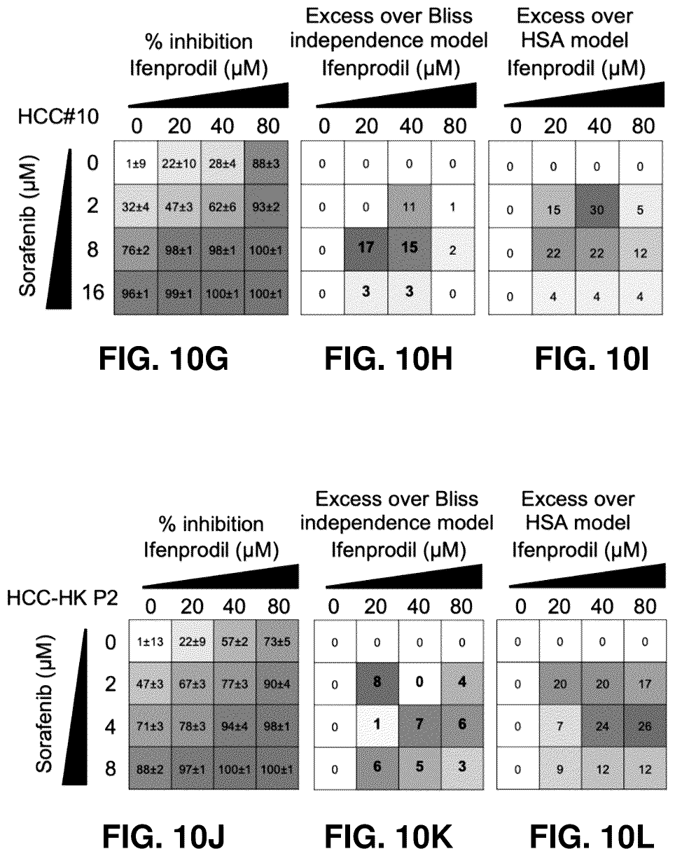

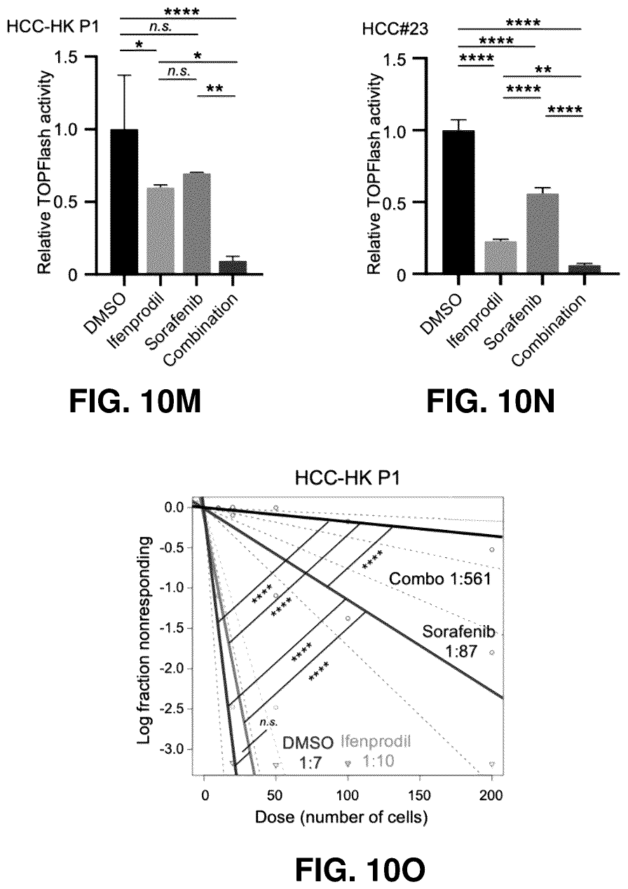

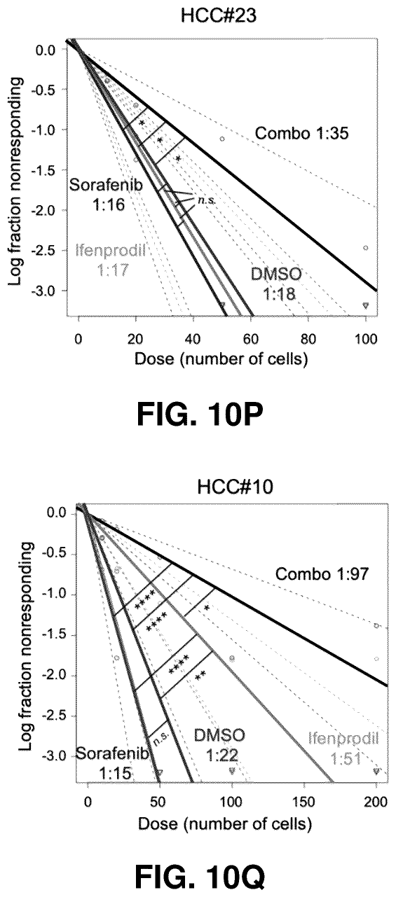

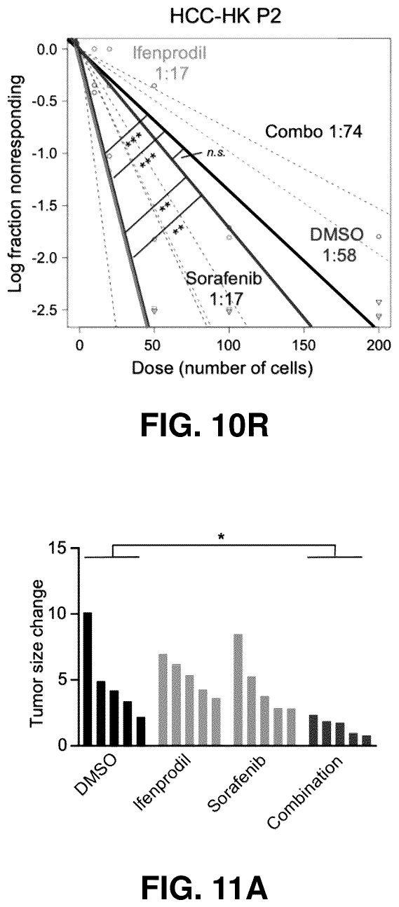

Human primary liver cancer-derived organoid culture models the pathophysiology of a growing tumor in vivo (Broutier et al. (2017), Nat Med 23:1424-35). A panel of HCC patient-derived organoids was used to evaluate inhibitions of growth and self-renewal brought by the drug combination. All HCC patient-derived organoids used in this study have been thoroughly characterized, either in-house or in previous studies (Huch et al. (2015), Cell 2015; 160:299-312), at both molecular and phenotypic levels, with comparison made against the original tissue samples. The HCC patient-derived organoids were characterized at the molecular and phenotypic levels with comparisons made against the original tissue samples. Bright-field images of patient derived non-tumor liver and HCC organoids grown and continuously passage in culture for over 4 months at a scale bar 500 μm were taken. H&E and immunohistochemical analysis of AFP, GPC3 and CK19 in tissue and organoid samples at a scale bar 200 μm was conducted Consistent with what was observed in the cell line models, synergy between IFEN and SOR on suppressing HCC growth was detected in all four tested organoids (FIGS. 10A-10L). Combined treatment of ifenprodil and sorafenib synergistically inhibited the growth of multiple HCC patient-derived organoids. Increased expression of phosphorylated IRE1-alpha, CHOP, phosphorylated-eIF2-alpha, and p21^{cip1}, as well as a decrease in Cdk2, and reduced WNT-signaling was detected in HCC-HK P1 and HCC #23 569 organoids treated with 20 μM ifenprodil and 8 μM 570 sorafenib for 3 days (FIGS. 10M and 10N). Furthermore, the drug combination, but not either drug alone, profoundly suppressed the ability of those HCC organoids to assemble tumor spheroids (FIGS. 100-10R).

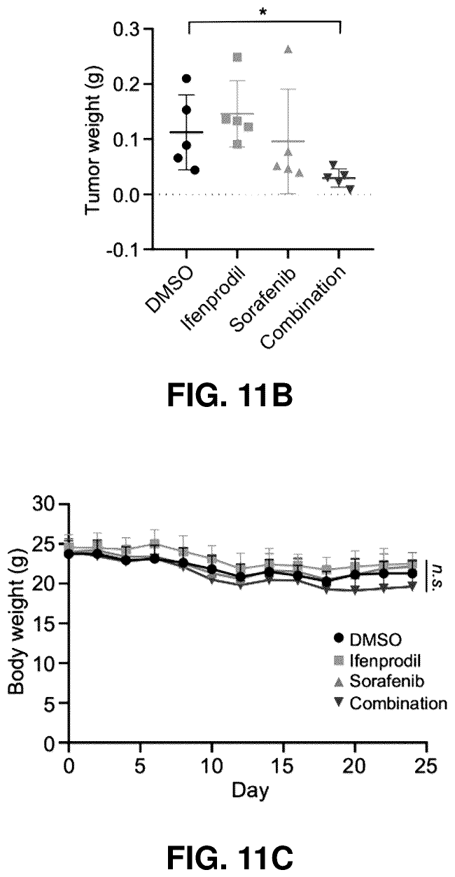

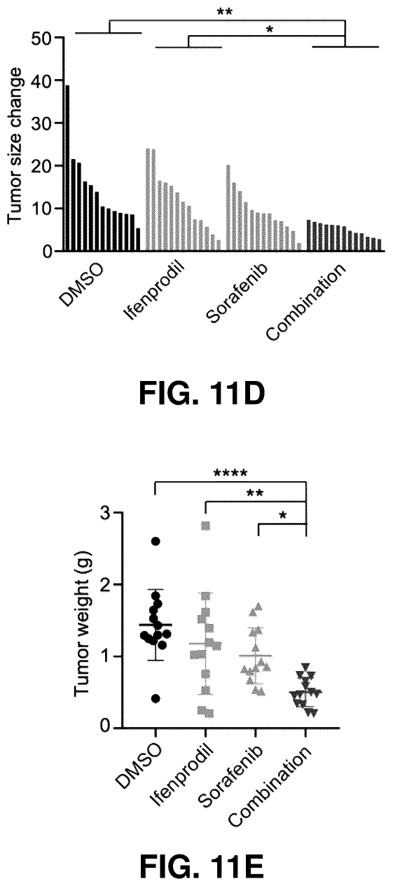

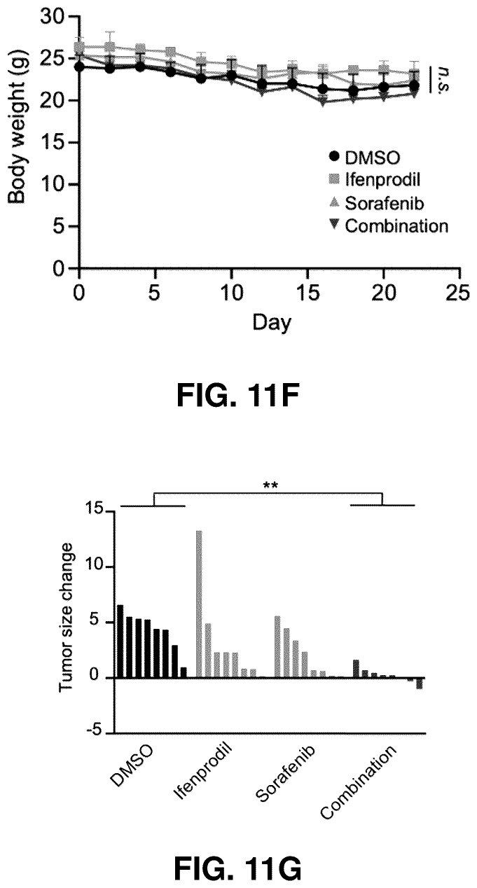

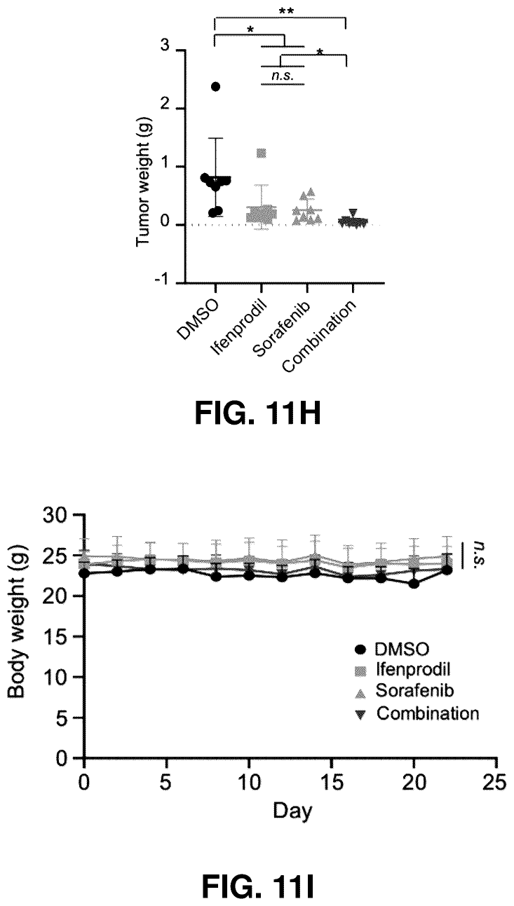

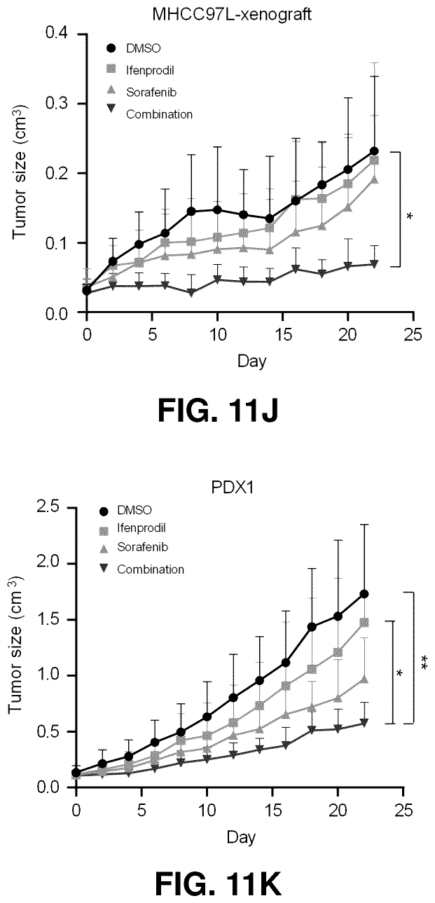

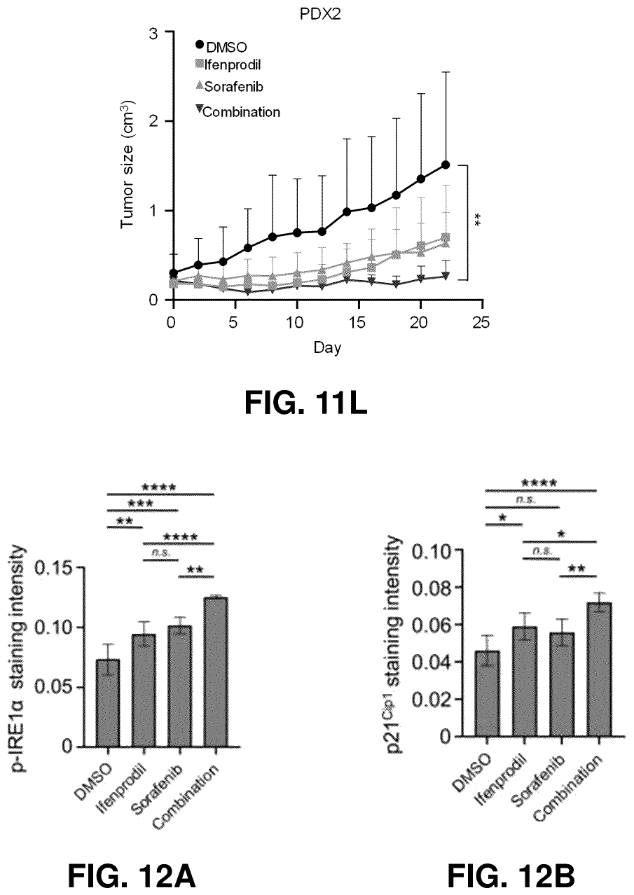

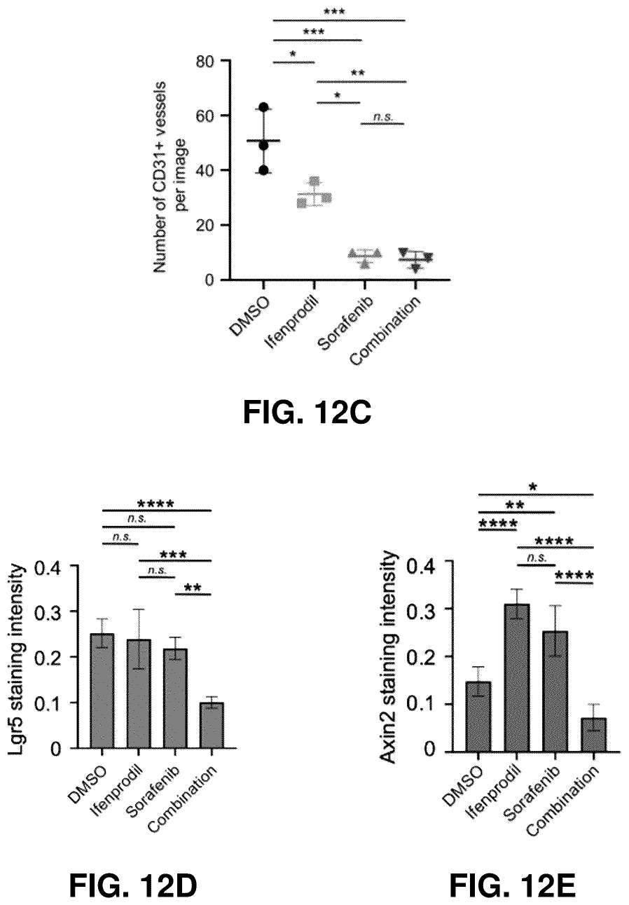

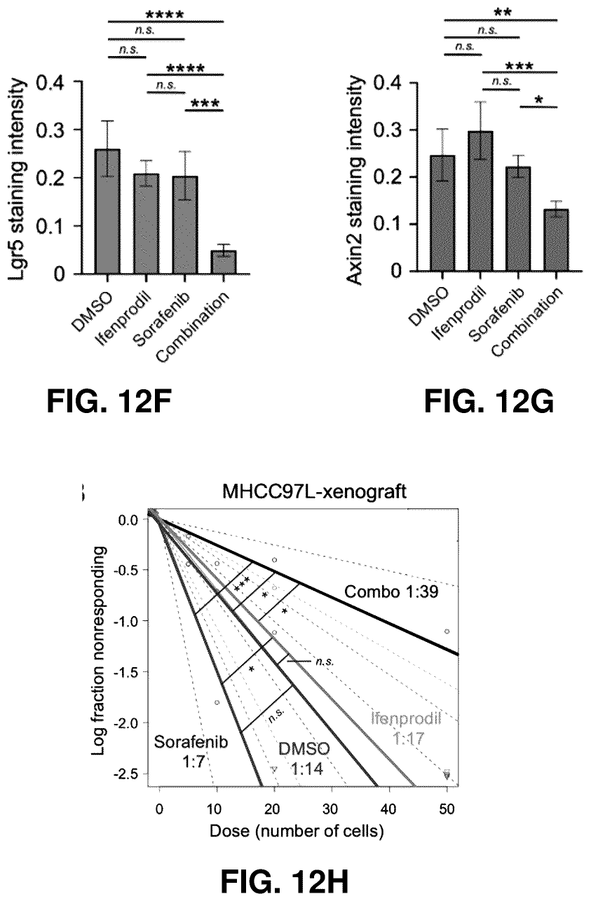

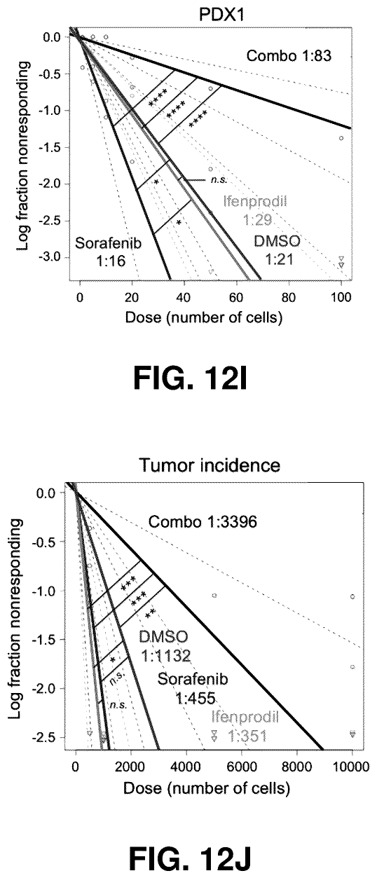

To determine the efficacy of the drug combination of IFEN and SOR in vivo, MHCC97L cell- and HCC patient-derived xenografts were implanted into mice. In all three xenografts, tumor weight was significantly suppressed in mice treated with the drug combination, but not the single drugs alone (FIGS. 11A-11L). There were signs of increased apoptosis (i.e., more small, swollen cells with hyper-eosinophilic cytoplasm) in tumors co-treated with IFEN and SOR (FIGS. 11A-11I), which is consistent with the increase in Annexin V-positive cells observed after the co-treatment in vitro (FIG. 9B). None of the mice showed signs of behavior abnormalities or significant changes in body weight (FIGS. 11A-11I). Corroborating with the drug response in vitro, treatment of the HCC patient-derived xenografts with the drug combination in vivo similarly led to an upregulation of UPR including increased expression of phosphorylated IRE1-alpha and CHOP and increase in p21^{ciP1} (FIGS. 12A-12C). Briefly, HCC patient-derived xenograft was subcutaneously injected in NOD/SCID mice and treated with 20 mg/kg ifenprodil, 28 mg/kg sorafenib, or combination of ifenprodil and sorafenib, for 21 days. Protein lysates were collected from the resected tumors and analyzed by Western blot. Immunohistochemical staining on tissue sections from the resected residual xenografts confirmed the reduced expressions of WNT-/stem cell-related Lgr5 and Axin2 proteins in xenografts of mice treated with the drug combination (FIGS. 12D-12G). Spheroid formation assay was performed on cells being harvested from the same residual xenografts to evaluate their self-renewal ability. Profound inhibition of the ability of the cells to assemble into spheroids was observed in the IFEN+SOR-treated group, but not in the vehicle-, IFEN-, or SOR-treated groups (FIGS. 12H and 12I). When the residual cells from the PDX xenografts were serially transplanted into secondary mouse recipients, the cells being treated previously with drug combination were less capable to reform tumors, while the ones being treated with only a single drug showed a more rapid rate and higher incidence of tumor formation (Tables 4 and 5). Combined treatment of ifenprodil and sorafenib decreased the repropagation capability of HCC cells in vivo. Residual tumor cells were harvested from the drug(s)-treated patient-derived xenografts and 50,000, 10,000, 5,000, 1,000, and 500 cells were transplanted into secondary NOD/SCID mouse recipients (n=6 per group). Average tumor latency and incidence were recorded, and tumor-initiating cell frequency was calculated. These results indicate the tumor-initiating property of cancer cells is depleted by the drug combination treatment. These results indicate the tumor-initiating property of HCC cells is depleted by the IFEN and SOR co-treatment.

TABLE 4
Average tumor latency days of patient-derived xenografts that
were serially transplanted into secondary mouse recipients

| Treatment | 50,000 | 10,000 | 5,000 | 1,000 | 500 |
|---|---|---|---|---|---|
| group | cells | cells | cells | cells | cells |
| DMSO | 21.84 | 20.33 | 25.17 | 32.2 | 31.2 |
| Ifenprodil | 24 | 21 | 31.33 | 28.33 | 33 |
| Sorafenib | 22.33 | 21.33 | 21.67 | 29 | 34 |
| Combination | 34.5 | 35 | 38 | 38.5 | 38.5 |

TABLE 5
Tumor incidence of patient-derived xenografts that were
serially transplanted into secondary mouse recipients

| Treatment | 50,000 | 10,000 | 5,000 | 1,000 | 500 | Estimated TIC |
|---|---|---|---|---|---|---|
| group | cells | cells | cells | cells | cells | Frequency |
| DMSO | 6/6 | 5/6 | 6/6 | 6/6 | 6/6 | 1:1132 |
| Ifenprodil | 6/6 | 6/6 | 6/6 | 6/6 | 4/6 | 1:351 |
| Sorafenib | 6/6 | 6/6 | 6/6 | 6/6 | 3/6 | 1:455 |
| Combination | 6/6 | 4/6 | 4/6 | 6/6 | 2/6 | 1:2607 |

### SUMMARY

The high incidence of recurrence and the limited efficacy of first-line drugs for liver cancer has prompted researchers and clinicians to explore new therapeutic options. The current study demonstrated a strategy to rapidly discover effective combination therapies and drug repurposing opportunities by inhibiting multiple druggable targets in the functional genomic screen. The screen results successfully guided us to validate the inhibitory effects on growth and self-renewal brought by combinatorial inhibition of NMDAR1 and FLT4/FGFR3, and the matching drug pair ifenprodil and sorafenib across all four HCC cell lines, four patient-derived organoids, and two xenograft models. This provides compelling evidence that the screening strategy is effective and could be applied as a standard approach in identifying potent therapeutic combinations. Importantly, the presented strategy can be easily adopted by many laboratories and used in studying various cancer cell types and other diseases.

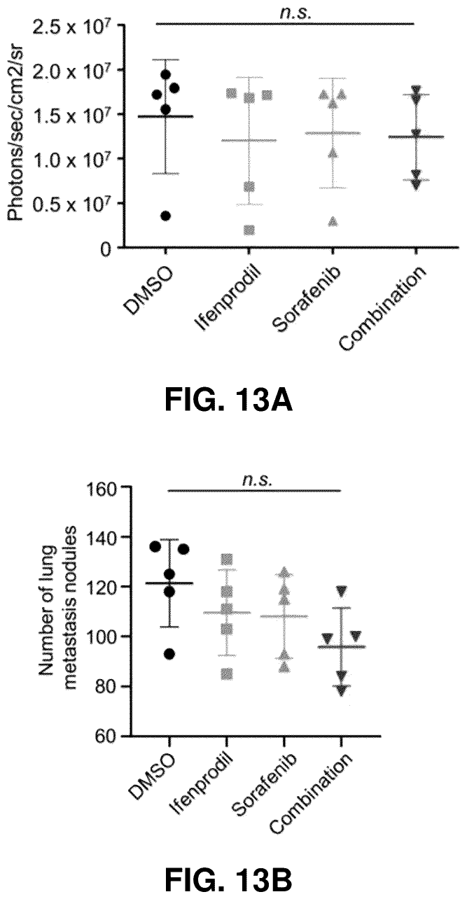

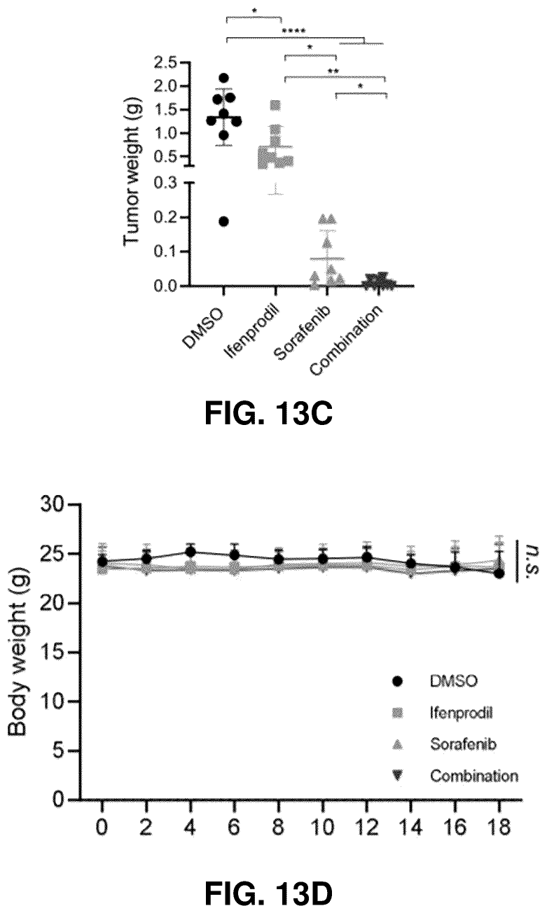

The current study established NMDAR inhibitors as a novel co-blockade agent for enhancing the efficacy of sorafenib in treating HCC. Previous studies have reported the role of NMDAR signaling in promoting growth, invasion, and metastasis in several cancer cell types but the role of NMDAR in HCC remains unclear. High NMDAR2B expression is associated with poor prognosis of pancreatic cancer, breast cancer, ovarian cancer, and glioblastoma (Li and Hanahan (2013), Cell 153:86-100). Pharmacological inhibition of NMDAR using MK-801 reduced tumor growth and invasiveness of pancreatic cancer cells, while genetic knockdown of NMDAR2B reduced breast-to-brain metastasis (Zeng et al. (2019), Nature 573:526-31). Based on TCGA LIHC database, it was noted that high NMDAR1 expression correlates with poor survival rate in HCC patients. Unlike in other cancers, inhibition of NMDAR using MK-801 or ifenprodil alone did not significantly suppress HCC growth. Nevertheless, unanticipated drug synergy between ifenprodil and sorafenib was revealed in the current study, in which the anti-cancer effects on inhibiting both HCC tumor growth and its ability to self-renew were greatly enhanced. The addition of IFEN to SOR offers an option to use a lower dose of SOR to reduce its toxicity to normal liver cells that express no or low level of NMDAR, while achieving greater or similar anti-cancer effects. Being able to lower the treatment dose of SOR could also reduce its discontinuation rate for HCC patients (Reiss et al. (2017) J Clin Oncol 35: pages 3575-81). Furthermore, in the current experiments, increased cancer stemness in the HCC mouse models after SOR treatment were observed, and addition of IFEN to the treatment reverted such phenotype, suggesting that the combination treatment may be useful for treating HCC cells that gain CSC properties after SOR treatment. The drug combination did not however inhibit the metastatic ability of HCC cells in a lung metastasis model in mouse (FIGS. 13A-13D). To evaluate HCC metastases, the RIL-175 cells used for establishing lung metastasis model were genetically modified to harbor p53 knockout and c-Myc overexpression, so that the cells could colonize and invade the lung tissues through tail vein injection; whereas the RIL-175 cells used for orthotopic injection into the liver do not harbor such modifications. Loss of p53 has been reported to drive metastasis in liver cancer (Chen et al (2007) Cancer Res; 67: pages 7589-96; Luo et al (2021) J Hepatol; 74: pages 96-10848). In the current study, it was also shown that combination treatment could decrease the expression of c-Myc, which is a WNT target gene. However, p53 expression or related signaling was not decreased as shown in the RNA-seq data. It is speculated that the drug combination treatment may not affect the activated signaling and metastatic phenotype resulted from p53 loss in RIL-175 cells, therefore inhibition of lung metastasis was not. As advanced HCC patients often present with aggressive clinical features such as tumor metastasis to different organs, it will be worthwhile to investigate if the drug combination treatment could inhibit the metastatic ability of HCC using human patient xenografts established from patients with different genetic mutations.

The mechanisms through which combining ifenprodil and sorafenib treatment leads to a stronger and synergistic suppression of HCC growth and self-renewal were investigated. Reports have shown that NMDAR inhibition could result in different cellular perturbations depending on the cellular context (Deutsch et al. (2013) Biomed Pharmacother 68: pages 493-6; Gynther et al. (2017), J Med Chem 60: pages 9885-904; Yamaguchi et al. (2013) BMC Cancer 13: page 468). The comparative transcriptomic analysis reveals that UPR was intensified after combining ifenprodil with sorafenib in treating MHCC97L cells. Across a panel of HCC cell lines and patient-derived organoids, strong activation of multiple UPR effectors were consistently detected at the protein level when these two drugs were co-applied. In addition, the unexpected synergy was detected between ifenprodil and sorafenib in downregulating both Cdk2 level and WNT signaling. The depletion of Cdk2 could account for the corresponding cell-cycle arrest at G1 phase being observed in IFEN+SOR-treated cells. On the other hand, WNT signaling is one of the key regulators of cancer sternness (Fodde and Brabletz (2007), Curr Opin Cell Biol. 19: pages 150-8). The suppressed WNT signaling, including the reduced expression of functional CSC marker protein Lgr5, in IFEN+SOR− treated cells could contribute to the reduced capacity of HCC cells to self-renew and re-grow into a tumor. These data lend support to the theory of CSC-mediated fueling of tumor growth and recurrence, as well as the rationale of exploiting cancer vulnerability via targeting molecular drivers operating in CSCs. The UPR induction could involve increased load of unfolded proteins at the endoplasmic reticulum (Fukaya et al. (2003) Proc Natl Acad Sci USA 100(8): pages 4855-4860), altered calcium homeostasis (Zeng et al. (2019), Nature 573:5 pages 26-31), and/or perturbed glutamate metabolism (Li and Hanahan (2013), Cell 153: pages 86-100).

Clinically, much research has been carried out in search for potent Cdk2 blockers and WNT inhibitors for cancer treatment. Cdk2 is hypothesized to be dispensable in the cell cycle of normally functioning cells but is important to the growth of cancer cells (Duensing et al. (2006) Oncogene 25: pages 2943-2949). However, a Cdk2 inhibitor is difficult to design given its high similarity to other essential Cdk proteins like Cdk1 (Wood et al. (2019), Cell Chem Biol 26: pages 121-30 e5), while non-selective Cdk inhibitors are toxic to most cells (Asghar et al. (2015), Nat Rev Drug Discov. 14: pages 130-46). On the other hand, many of the WNT inhibitors being developed are still undergoing pre-clinical or early clinical trials to evaluate their efficacy and safety (Kahn (2014), Nat Rev Drug Discov. 13: pages 513-32). The results uncovered ifenprodil as a safe drug candidate that acts to augment sorafenib's anti-cancer effects and repress Cdk2 and WNT signaling in HCC. In addition to HCC, sorafenib is indicated for treating other cancers including advanced renal cell carcinoma, thyroid cancer, and FLT3-ITD positive acute myeloid leukemia, in all which Cdk2 and/or WNT signaling have also been implicated in their tumorigenesis and recurrence (Fendler et al. (2020), Nat Commun. 11: page 929; Jiang et al. (2018), Clin Cancer Res. 24: pages 2417-29; Sastre-Perona and Santisteban (2012), Front Endocrinol (Lausanne) 2012 3: page 31; Wang et al. (2015), Mol Cancer Res. 13: pages 1567-77).

In translating the genetic targets that were identified in the current screen into matching drugs to be readily tested, references on the DrugBank (Wishart et al. (2018), Nucleic Acids Res. 46: pages D1074-D82) were used to select drugs with known target information and safety profile to maximize the repurposing opportunities. Sorafenib and lenvatinib were used as the matching inhibitors to target FLT4 and FGFR3 because they are the first-line drugs for treating HCC. Ifenprodil was chosen to target NMDAR as it is an approved inhibitor drug with known mechanism of action and was tested safe in human. Ifenprodil was originally developed as a drug to treat peripheral circulatory disorders, and there have been ongoing investigations on repurposing ifenprodil for treating patients with idiopathic pulmonary fibrosis and lung injury associated with COVID-19 infection (NCT04382924). In the current study, strong anti-cancer effects were brought by combined treatment of ifenprodil and sorafenib using HCC xenograft models, while no toxicity was detected in the drug-treated mice.

We claim:

1. A pharmaceutical composition comprising an effective amount of the combination of an N-methyl-D-aspartate receptor (NMDAR) inhibitor and a kinase inhibitor, wherein administration of the pharmaceutical composition reduces cancer cell proliferation or reduces cancer cell viability, or reduces both cancer cell viability and proliferation in a subject with cancer to a greater degree than administering to the subject the same amount of the NMDAR inhibitor alone or the same amount of the kinase inhibitor alone.

2. The pharmaceutical composition of claim 1, wherein the reduction in cancer cell proliferation and/or viability in the subject with cancer is more than the additive reduction achieved by administering the NMDAR inhibitor alone or the kinase inhibitor alone.

3. The pharmaceutical composition of claim 1, wherein the NMDAR inhibitor is selected from the group consisting of ifenprodil, 4-Chlorokynurenine, 7-Chlorokynurenic acid, Kynurenic acid, Phenylalanine, AP5, AP7, CGP-37849, Kaitocephalin, LY-235959, Midafotel, PEAQX, Perzinfotel, Selfotel; dizocilpine, Delucemine, Dextromethorphan, Dextrorphan, Dexanabinol, Diphenidine, Dizocilpine, Esketamine, Ketamine, Lanicemine, Memantine, Methoxetamine, Phencyclidine, Tiletamine, and Amantadine, Diethyl ether, Eliprodil, Hodgkinsine, Nitrous oxide, Psychotridine, Traxoprodil, Xenon, Atomoxetine, Dextropropoxyphene, Ethanol, Guaifenesin, Huperzine A, Ibogaine, Ketobemidone, Methadone, Minocycline, and Tramadol.

4. The pharmaceutical composition of claim 1, wherein the NMDAR inhibitor is ifenprodil, a pharmaceutically acceptable salt of ifenprodil, a prodrug, analog, or derivative of ifenprodil, or a pharmaceutically acceptable salt of a prodrug, analog, or derivative of ifenprodil.

5. The pharmaceutical composition of claim 4, wherein the dosage of ifenprodil is 1 mg-100 mg.

6. The pharmaceutical composition of claim 1, wherein the kinase inhibitor is a receptor tyrosine kinase inhibitor.

7. The pharmaceutical composition of claim 6, wherein receptor tyrosine kinase inhibitor is an inhibitor of Fibroblast Growth Factor Receptor or Fms-related tyrosine kinase 4.

8. The pharmaceutical composition of claim 1, wherein the kinase inhibitor is selected from the group consisting of sorafenib, lenvatinib, infigratinib, erdafitinib, SAR131675, crizotinib, ceritinib, alectinib, brigatinib, bosutinib, dasatinib, imatinib, nilotinib, vemurafenib, dabrafenib, ibrutinib, palbociclib, ribociclib, cabozantinib, gefitinib, erlotinib, lapatinib, vandetanib, afatinib, osimertinib, ruxolitinib, tofacitinib, trametinib, axitinib, toceranib, nintedanib, pazopanib, regorafenib, sunitinib, dacomitinib, and ponatinib.

9. The pharmaceutical composition of claim 1, wherein the kinase inhibitor is sorafenib.

10. The pharmaceutical composition of claim 9, wherein the dosage of sorafenib is 100 mg to 1000 mg.

11. The pharmaceutical composition of claim 1, wherein the cancer cells are hepatocellular carcinoma.

12. The pharmaceutical composition of claim 1, wherein the cancer cells have aberrant Wnt/β-catenin signaling and/or Cdk2 signaling compared to non-cancerous cells.

13. A method of treating cancer comprising administering to a subject with cancer an effective amount of an N-methyl-D-aspartate receptor (NMDAR) inhibitor in combination with an effective amount of a kinase inhibitor, wherein administration of the combination the NMDAR inhibitor and the kinase inhibitor reduces cancer cell proliferation and/or viability in the subject with cancer to a greater degree than administering to the subject the same amount of NMDAR inhibitor alone or the same amount of the kinase inhibitor alone.

14. The method of claim 13, wherein the reduction in cancer cell proliferation and/or viability in the subject with cancer is more than the additive reduction achieved by administering the NMDAR inhibitor alone or the kinase inhibitor alone.

15. The method of claim 13, wherein the NMDAR inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the kinase inhibitor to the subject.

16. The method of claim 13, wherein the kinase inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the NMDAR inhibitor to the subject.

17. The method of claim 13, further comprising surgery or radiation therapy.

18. The method of claim 13, wherein the cancer to be treated is characterized by expression of genes involved in cancer stemness, Wnt/β-catenin signaling pathway, and/or Cdk2 signaling pathway.

19. The method of claim 13, wherein one or more genes involved in cancer sternness, Wnt/β-catenin signaling pathway, and/or Cdk2 signaling pathway are selected from the group consisting of Lgr5, Axin2, HES1, AFP, NES, and Tert.

20. The method of claim 13, wherein the cancer is characterized by down regulation of expression of one or more genes selected from the group consisting of Lgr5, Axin2, HES1, AFP, NES, and Tert following treatment.

21. The method of claim 13, wherein the cancer is characterized by up-regulation of unfolded protein response effectors including phosphorylated IRE1-alpha and/or C/EBP Homologous Protein (CHOP) following treatment.

22. The method of claim 13, further comprising the step of selecting a subject having a cancer characterized by overexpression of one or more genes involved in cancer stemness, Wnt/β-catenin signaling pathway, and/or Cdk2 signaling pathway.

23. The method of claim 13, wherein the NMDAR inhibitor and the kinase inhibitor are administered in an amount effective to reduce the serum concentration of one or more of Lens culinaris-reactive AFP, Golgi Protein 73, Asialo-alpha-acid glycoprotein, laminin, neopterin, and Glypican-3 compared to the serum concentration of one or more of Lens culinaris-reactive AFP, Golgi Protein 73, Asialo-alpha-acid glycoprotein, laminin, neopterin, and Glypican-3 prior to treatment.
